(19) United States
(12) Patent Application Publication
(10) Pub. No.: US 2022/0388957 A1
(43) Pub. Date: Dec. 8, 2022

(54) COMPOUNDS AND COMPOSITIONS FOR TREATING CONDITIONS ASSOCIATED WITH STING ACTIVITY

(71) Applicant: IFM Due, Inc., Boston, MA (US)

(72) Inventors: Hans Martin Seidel, Concord, CA (US); William R. Roush, Boston, MA (US); Shankar Venkatraman, Lansdale, PA (US)

(21) Appl. No.: 17/422,397

(22) PCT Filed: Jan. 16, 2020
(86) PCT No.: PCT/US2020/013824

§ 371 Date: Jul. 12, 2021

Related U.S. Application Data
(60) Provisional application No. 62/861,702, filed on Jun. 14, 2019.
(60) Provisional application No. 62/793,623, filed on Jan. 17, 2019.

(51) Int. Cl. C07D 209/40; A61K 31/404; C07D 471/04; A61K 31/437; A61K 31/415; C07D 231/42; C07D 498/04; A61K 31/5383; A61K 31/4439; A61K 31/4545; C07D 401/12; C07D 401/14; C07D 403/12; C07D 405/12; C07D 409/12

(52) U.S. Cl. CPC C07D 209/40; A61K 31/404; C07D 471/04; A61K 31/437; A61K 31/415; C07D 231/42; C07D 498/04; A61K 31/5383; A61K 31/4439; A61K 31/4545; C07D 401/12; C07D 401/14; C07D 403/12; C07D 405/12; C07D 409/12

(57) ABSTRACT

This disclosure features chemical entities (e.g., a compound or a pharmaceutically acceptable salt, and/or hydrate, and/or cocrystal, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

### CROSS REFERENCE TO RELATED APPLICATIONS

This application claims the benefit of U.S. Provisional Application Ser. No. 62/793,623, filed on Jan. 17, 2019; and U.S. Provisional Application Ser. No. 62/861,702, filed on Jun. 14, 2019; each of which is incorporated herein by reference in its entirety.

### TECHNICAL FIELD

This disclosure features chemical entities (e.g., a compound or a pharmaceutically acceptable salt, and/or hydrate, and/or cocrystal, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

### BACKGROUND

STING, also known as transmembrane protein 173 (TMEM173) and MPYS/MITA/ERIS, is a protein that in humans is encoded by the TMEM173 gene. STING has been shown to play a role in innate immunity. STING induces type I interferon production when cells are infected with intracellular pathogens, such as viruses, mycobacteria and intracellular parasites. Type I interferon, mediated by STING, protects infected cells and nearby cells from local infection in an autocrine and paracrine manner.

The STING pathway is pivotal in mediating the recognition of cytosolic DNA. In this context, STING, a transmembrane protein localized to the endoplasmic reticulum (ER), acts as a second messenger receptor for 2′, 3′ cyclic GMP-AMP (hereafter cGAMP), which is produced by cGAS after dsDNA binding. In addition, STING can also function as a primary pattern recognition receptor for bacterial cyclic dinucleotides (CDNs) and small molecule agonists. The recognition of endogenous or prokaryotic CDNs proceeds through the carboxy-terminal domain of STING, which faces into the cytosol and creates a V-shaped binding pocket formed by a STING homodimer. Ligand-induced activation of STING triggers its re-localization to the Golgi, a process essential to promote the interaction of STING with TBK1. This protein complex, in turn, signals through the transcription factors IRF-3 to induce type I interferons (IFNs) and other co-regulated antiviral factors. In addition, STING was shown to trigger NF-κB and MAP kinase activation. Following the initiation of signal transduction, STING is rapidly degraded, a step considered important in terminating the inflammatory response.

Excessive activation of STING is associated with a subset of monogenic autoinflammatory conditions, the so-called type I interferonopathies. Examples of these diseases include a clinical syndrome referred to as STING-associated vasculopathy with onset in infancy (SAVI), which is caused by gain-of-function mutations in TMEM173 (the gene name of STING). Moreover, STING is implicated in the pathogenesis of Aicardi-Goutières Syndrome (AGS) and genetic forms of lupus. As opposed to SAVI, it is the dysregulation of nucleic acid metabolism that underlies continuous innate immune activation in AGS. Apart from these genetic disorders, emerging evidence points to a more general pathogenic role for STING in a range of inflammation-associated disorders such as systemic lupus erythematosus, rheumatoid arthritis and cancer. Thus, small molecule-based pharmacological interventions into the STING signaling pathway hold significant potential for the treatment of a wide spectrum of diseases

### SUMMARY

This disclosure features chemical entities (e.g., a compound or a pharmaceutically acceptable salt, and/or hydrate, and/or cocrystal, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

An “antagonist” of STING includes compounds that, at the protein level, directly bind or modify STING such that an activity of STING is decreased, e.g., by inhibition, blocking or dampening agonist-mediated responses, altered distribution, or otherwise. STING antagonists include chemical entities, which interfere or inhibit STING signaling.

In one aspect, compounds of Formula (I), or a pharmaceutically acceptable salt thereof, are featured:

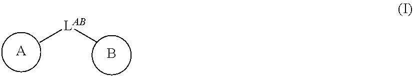

in which A, B, and L^{AB} can be as defined anywhere herein.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for inhibiting (e.g., antagonizing) STING activity are featured that include contacting STING with a chemical entity described herein (e.g., a compound described generically or specifically herein or a pharmaceutically acceptable salt thereof or compositions containing the same). Methods include in vitro methods, e.g., contacting a sample that includes one or more cells comprising STING (e.g., innate immune cells, e.g., mast cells, macrophages, dendritic cells (DCs), and natural killer cells) with the chemical entity. Methods can also include in vivo methods; e.g., administering the chemical entity to a subject (e.g., a human) having a disease in which increased (e.g., excessive) STING signaling contributes to the pathology and/or symptoms and/or progression of the disease.

In one aspect, methods of treating a condition, disease or disorder ameliorated by antagonizing STING are featured, e.g., treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). The methods include administering to a subject in need of such treatment an effective amount of a chemical entity described herein (e.g., a compound described generically or specifically herein or a pharmaceutically acceptable salt thereof or compositions containing the same).

In another aspect, methods of treating cancer are featured that include administering to a subject in need of such treatment an effective amount of a chemical entity described herein (e.g., a compound described generically or specifically herein or a pharmaceutically acceptable salt thereof or compositions containing the same).

In a further aspect, methods of treating other STING-associated conditions are featured, e.g., type I interferonopathies (e.g., STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis. The methods include administering to a subject in need of such treatment an effective amount of a chemical entity described herein (e.g., a compound described generically or specifically herein or a pharmaceutically acceptable salt thereof or compositions containing the same).

In another aspect, methods of suppressing STING-dependent type I interferon production in a subject in need thereof are featured that include administering to the subject an effective amount of a chemical entity described herein (e.g., a compound described generically or specifically herein or a pharmaceutically acceptable salt thereof or compositions containing the same).

In a further aspect, methods of treating a disease in which increased (e.g., excessive) STING activation (e.g., STING signaling) contributes to the pathology and/or symptoms and/or progression of the disease are featured. The methods include administering to a subject in need of such treatment an effective amount of a chemical entity described herein (e.g., a compound described generically or specifically herein or a pharmaceutically acceptable salt thereof or compositions containing the same).

In another aspect, methods of treatment are featured that include administering an effective amount of a chemical entity described herein (e.g., a compound described generically or specifically herein or a pharmaceutically acceptable salt thereof or compositions containing the same) to a subject; wherein the subject has (or is predisposed to have) a disease in which increased (e.g., excessive) STING activation (e.g., STING signaling) contributes to the pathology and/or symptoms and/or progression of the disease.

In a further aspect, methods of treatment that include administering to a subject a chemical entity described herein (e.g., a compound described generically or specifically herein or a pharmaceutically acceptable salt thereof or compositions containing the same), wherein the chemical entity is administered in an amount effective to treat a disease in which increased (e.g., excessive) STING activation (e.g., STING signaling) contributes to the pathology and/or symptoms and/or progression of the disease, thereby treating the disease.

Embodiments can include one or more of the following features.

The chemical entity can be administered in combination with one or more additional therapeutic agents and/or regimens. For examples, methods can further include administering one or more (e.g., two, three, four, five, six, or more) additional agents.

The chemical entity can be administered in combination with one or more additional therapeutic agents and/or regimens that are useful for treating other STING-associated conditions, e.g., type I interferonopathies (e.g., STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis.

The chemical entity can be administered in combination with one or more additional cancer therapies (e.g., surgery, radiotherapy, chemotherapy, toxin therapy, immunotherapy, cryotherapy or gene therapy, or a combination thereof, e.g., chemotherapy that includes administering one or more (e.g., two, three, four, five, six, or more) additional chemotherapeutic agents. Non-limiting examples of additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

The subject can have cancer; e.g., the subject has undergone and/or is undergoing and/or will undergo one or more cancer therapies.

Non-limiting examples of cancer include melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma. In certain embodiments, the cancer can be a refractory cancer.

The chemical entity can be administered intratumorally.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “STING” is meant to include, without limitation, nucleic acids, polynucleotides, oligonucleotides, sense and antisense polynucleotide strands, complementary sequences, peptides, polypeptides, proteins, homologous and/or orthologous STING molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

The term “halo” refers to fluoro (F), chloro (Cl), bromo (Br), or iodo (I).

The term “alkyl” refers to a hydrocarbon chain that may be a straight chain or branched chain, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “alkylene” refers to a divalent alkyl (e.g., —CH₂—).

The term “alkenyl” refers to a hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it.

The term “alkynyl” refers to a hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein includes cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

The term “cycloalkenyl” as used herein includes partially unsaturated cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkenyl group may be optionally substituted. Examples of cycloalkenyl groups include, without limitation, cyclopentenyl, cyclohexenyl, cycloheptenyl, and cyclooctenyl. Cycloalkenyl groups may have any degree of saturation provided that none of the rings in the ring system are aromatic; and the cycloalkenyl group is not fully saturated overall. Cycloalkenyl may include multiple fused and/or bridged and/or spirocyclic rings.

The term “heteroaryl”, as used herein, means a mono-, bi-, tri- or polycyclic group having 5 to 20 ring atoms, alternatively 5, 6, 9, 10, or 14 ring atoms; and having 6, 10, or 14 pi electrons shared in a cyclic array; wherein at least one ring in the system is aromatic (but does not have to be a ring which contains a heteroatom, e.g. tetrahydroisoquinolinyl, e.g., tetrahydroquinolinyl), and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

The term “heterocyclyl” refers to a mon-, bi-, tri-, or polycyclic nonaromatic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

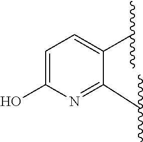 [SMILES: *c1ccc(O)nc1*]

encompasses the tautomeric form containing the moiety:

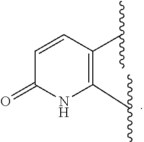 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The details of one or more embodiments of the invention are set forth in the accompanying drawings and the description below. Other features and advantages of the invention will be apparent from the description and drawings, and from the claims.

### DETAILED DESCRIPTION

This disclosure features chemical entities (e.g., a compound or a pharmaceutically acceptable salt, and/or hydrate, and/or cocrystal, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

Formula I Compounds

In one aspect, compounds of Formula (I), or a pharmaceutically acceptable salt thereof, are featured:

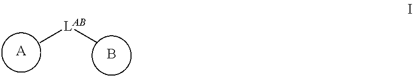

or a pharmaceutically acceptable salt thereof or a tautomer thereof,
wherein:
L^{AB} is —N(R^{N})S(O)₂—*, —N(R^{N})S(O)₂—(W^{AB1}—W^{AB2}—W^{AB3})—*, —S(O)₂N(R^{N})—*, wherein the asterisk represents point of attachment to B;
W^{AB1} is C₁₋₃ alkylene optionally substituted with from 1-4 independently selected R^{a};
W^{AB2} is a bond, —O—, —NR^{N}, or —S—;
W^{AB3} is a bond or C₁₋₃ alkylene optionally substituted with from 1-4 independently selected R^{a};
A is selected from the group consisting of:
(i) heteroaryl including from 5-6 ring atoms, wherein from 1-4 ring atoms are heteroatoms, each independently selected from the group consisting of N, N(H), N(R¹), N(R²), O, and S, and wherein from 1-5 ring atoms are carbon atoms, each independently selected from the group consisting of C, CH, CR¹, and CR³; provided that at least one ring atom is substituted with R¹; and
(ii) heteroaryl including from 7-20 ring atoms, wherein from 1-4 ring atoms are heteroatoms, each independently selected from the group consisting of N, N(H), N(R¹), N(R²), O, and S(O)₀₋₂, and wherein from 3-19 ring atoms are carbon atoms, each independently selected from the group consisting of C, CH, CH₂, CR¹, CHR¹, C(R¹)₂, CR³, CHR³, and C(R³)₂;

### B is:

(a) C₁₋₁₅ alkyl which is optionally substituted with from 1-6 R^{a};
(b) C₃₋₂₀ cycloalkyl, which is optionally substituted with from 1-4 R^{b};
(c) C₆₋₂₀ aryl optionally substituted with from 1-4 R^{c};
(d) heteroaryl including from 5-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
(e) heterocyclyl including from 3-16 ring atoms, wherein from 1-3 ring atoms are heteroatoms, each independently selected from the group consisting of N(H), N(R^{d}), O, and S(O)₀₋₂ and wherein the heterocyclyl ring is optionally substituted with from 1-4 independently selected R^{b};

### R^{N} is:

### (i) H, or

(ii) C₁₋₆ alkyl optionally substituted with from 1-3 R^{a},

### R¹ is:

(i) —(U¹)_{q}—U², wherein:
q is 0 or 1;
U¹ is C₁₋₆ alkylene, which is optionally substituted with from 1-6 R^{a}; and
U² is:
   (a) C₃₋₁₂ cycloalkyl, which is optionally substituted with from 1-4 R^{b},
   (b) C₆₋₁₀ aryl, which is optionally substituted with from 1-4 R^{c};
   (c) heteroaryl including from 5-20 ring atoms, wherein from 1-4 ring atoms are heteroatoms, each independently selected from the group consisting of N, N(H), N(R^{d}), O, S(O)₀₋₂, and wherein the heteroaryl ring is optionally substituted with from 1-4 independently selected R^{c}, or
   (d) heterocyclyl including from 3-12 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

### OR

(ii) C₁₋₁₀ alkyl, which is optionally substituted with from 1-6 independently selected R^{a};
each occurrence of R² is independently selected from the group consisting of:
(i) C₁₋₆ alkyl, which is optionally substituted with from 1-2 independently selected R^{a};
(ii) C₃₋₆ cycloalkyl;
(iii) heterocyclyl including from 3-10 ring atoms, wherein from 1-3 ring atoms are heteroatoms, each independently selected from the group consisting of N, N(H), N(R^{d}), O, and S(O)₀₋₂.
(iv) —C(O)(C₁₋₄ alkyl);
(v) —C(O)O(C₁₋₄ alkyl);

### (vi) —CON(R′)(R″);

(vii) —S(O)₁₋₂(NR′R″);
(viii) —S(O)₁₋₂(C₁₋₄ alkyl);

### (ix) —OH; and

(x) C₁₋₄ alkoxy;
each occurrence of R³ is independently selected from the group consisting of halo, cyano, C₂₋₆ alkenyl, C₂₋₆ alkynyl, C₁₋₄ alkoxy, C₁₋₄ haloalkoxy, —S(O)₁₋₂(C₁₋₄ alkyl), —NR^{e}R^{f}, —OH, oxo, —S(O)₁₋₂(NR′R″), —C₁₋₄ thioalkoxy, —NO₂, —C(═O)(C₁₋₄ alkyl), —C(═O)O(C₁₋₄ alkyl), —C(═O)OH, and —C(═O)N(R′)(R″);
each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;
each occurrence of R^{b} is independently selected from the group consisting of: C₁₋₁₀ alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};
each occurrence of R^{c} is independently selected from the group consisting of:
(a) halo;
(b) cyano;
(c) C₁₋₁₅ alkyl which is optionally substituted with from 1-6 independently selected R^{a};
(d) C₂₋₆ alkenyl;
(e) C₂₋₆ alkynyl;
(g) C₁₋₄ alkoxy optionally substituted with from 1-3 independently selected R^{a};
(h) C₁₋₄ haloalkoxy;
(i) —S(O)₁₋₂(C₁₋₄ alkyl);
(j) —NR^{e}R^{f};

### (k) —OH;

### (l) —S(O)₁₋₂(NR′R″);

(m) —C₁₋₄ thioalkoxy;

### (n) —NO₂;

(o) —C(═O)(C₁₋₄ alkyl);
(p) —C(═O)O(C₁₋₄ alkyl);

### (q) —C(═O)OH;

### (r) —C(═O)N(R′)(R″); and

(s) -L¹-L²-R^{h}.
R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;
each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R′ and R″), which are each independently selected from the group consisting of N(R^{d}), NH, O, and S;
-L¹ is a bond or C₁₋₃ alkylene;
-L² is —O—, —N(H)—, —S—, or a bond;
R^{h} is selected from:
C₃₋₈ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂ wherein the heterocyclyl is optionally substituted with from 1-4 substituents independently selected from the group consisting of halo, C₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 substituents independently selected from the group consisting of halo, C₁₋₄ alkyl, and C₁₋₄ haloalkyl; and
C₆₋₁₀ aryl, which is optionally substituted with from 1-4 substituents independently selected from the group consisting of halo, C₁₋₄ alkyl, or C₁₋₄ haloalkyl; and
   each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(R^{d}), O, and S.

In one aspect, compounds of Formula (I), or a pharmaceutically acceptable salt thereof, are featured:

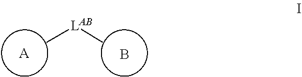

or a pharmaceutically acceptable salt thereof or a tautomer thereof,
wherein:
L^{AB} is —N(R^{N})S(O)₂—* or —S(O)₂N(R^{N})—*, wherein the asterisk represents point of attachment to B;
A is selected from the group consisting of:
(i) heteroaryl including from 5-6 ring atoms, wherein from 1-4 ring atoms are heteroatoms, each independently selected from the group consisting of N, N(H), N(R¹), N(R²), O, and S, and wherein from 1-5 ring atoms are carbon atoms, each independently selected from the group consisting of C, CH, CR¹, and CR³; provided that at least one ring atom is substituted with R¹; and
(ii) heteroaryl including from 7-20 ring atoms, wherein from 1-4 ring atoms are heteroatoms, each independently selected from the group consisting of N, N(H), N(R¹), N(R²), O, and S(O)₀₋₂, and wherein from 3-19 ring atoms are carbon atoms, each independently selected from the group consisting of C, CH, CH₂, CR¹, CHR¹, C(R¹)², CR³, CHR³, and C(R³)₂;

### B is:

(a) C₁₋₁₅ alkyl which is optionally substituted with from 1-6 R^{a};
(b) C₃₋₂₀ cycloalkyl, which is optionally substituted with from 1-4 R^{b};
(c) C₆₋₂₀ aryl optionally substituted with from 1-4 R^{c};
(d) heteroaryl including from 5-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
(e) heterocyclyl including from 3-16 ring atoms, wherein from 1-3 ring atoms are heteroatoms, each independently selected from the group consisting of N(H), N(R^{d}), O, and S(O)₀₋₂ and wherein the heterocyclyl ring is optionally substituted with from 1-4 independently selected R^{b};

### R^{N} is:

### (i) H, or

(ii) C₁₋₆ alkyl optionally substituted with from 1-3 R^{a},

### R¹ is:

(i) —(U¹)_{q}—U², wherein:
q is 0 or 1;
U¹ is C₁₋₆ alkylene, which is optionally substituted with from 1-6 R^{a}; and
U² is:
   (a) C₃₋₁₂ cycloalkyl, which is optionally substituted with from 1-4 R^{b},
   (b) C₆₋₁₀ aryl, which is optionally substituted with from 1-4 R^{c};
   (c) heteroaryl including from 5-20 ring atoms, wherein from 1-4 ring atoms are heteroatoms, each independently selected from the group consisting of N, N(H), N(R^{d}), O, S(O)₀₋₂, and wherein the heteroaryl ring is optionally substituted with from 1-4 independently selected R^{c}, or
   (d) heterocyclyl including from 3-12 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

### OR

(ii) C₁₋₁₀ alkyl, which is optionally substituted with from 1-6 independently selected R^{a};
each occurrence of R² is independently selected from the group consisting of:
(i) C₁₋₆ alkyl, which is optionally substituted with from 1-2 independently selected R^{a};
(ii) C₃₋₆ cycloalkyl;
(iii) heterocyclyl including from 3-10 ring atoms, wherein from 1-3 ring atoms are heteroatoms, each independently selected from the group consisting of N, N(H), N(R^{d}), O, and S(O)₀₋₂.
(iv) —C(O)(C₁₋₄ alkyl);
(v) —C(O)O(C₁₋₄ alkyl);

### (vi) —CON(R′)(R″);

(vii) —S(O)₁₋₂(NR′R″);
(viii) —S(O)₁₋₂(C₁₋₄ alkyl);

### (ix) —OH; and

(x) C₁₋₄ alkoxy;
each occurrence of R³ is independently selected from the group consisting of halo, cyano, C₂₋₆ alkenyl, C₂₋₆ alkynyl, C₁₋₄ alkoxy, C₁₋₄ haloalkoxy, —S(O)₁₋₂(C₁₋₄ alkyl), —NR^{e}R^{f}, —OH, oxo, —S(O)₁₋₂(NR′R″), —C₁₋₄ thioalkoxy, —NO₂, —C(═O)(C₁₋₄ alkyl), —C(═O)O(C₁₋₄ alkyl), —C(═O)OH, and —C(═O)N(R′)(R″);
each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;
each occurrence of R^{b} is independently selected from the group consisting of: C₁₋₁₀ alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};
each occurrence of R^{c} is independently selected from the group consisting of:
(a) halo;
(b) cyano;
(c) C₁₋₁₅ alkyl which is optionally substituted with from 1-6 independently selected R^{a};
(d) C₂₋₆ alkenyl;
(e) C₂₋₆ alkynyl;
(g) C₁₋₄ alkoxy;
(h) C₁₋₄ haloalkoxy;
(i) —S(O)₁₋₂(C₁₋₄ alkyl);
(j) —NR^{e}R^{f};

### (k) —OH;

### (l) —S(O)₁₋₂(NR′R″);

(m) —C₁₋₄ thioalkoxy;

### (n) —NO₂;

(o) —C(═O)(C₁₋₄ alkyl);
(p) —C(═O)O(C₁₋₄ alkyl);

### (q) —C(═O)OH;

### (r) —C(═O)N(R′)(R″); and

(s) -L¹-L²-R^{h}.

R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R′ and R″), which are each independently selected from the group consisting of N(R^{d}), NH, O, and S;
-L¹ is a bond or C₁₋₃ alkylene;
-L² is —O—, —N(H)—, —S—, or a bond;
R^{h} is selected from:
C₃₋₈ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂ wherein the heterocyclyl is optionally substituted with from 1-4 substituents independently selected from the group consisting of halo, C₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 substituents independently selected from the group consisting of halo, C₁₋₄ alkyl, and C₁₋₄ haloalkyl; and
C₆₋₁₀ aryl, which is optionally substituted with from 1-4 substituents independently selected from the group consisting of halo, C₁₋₄ alkyl, or C₁₋₄ haloalkyl; and
   each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(R^{d}), O, and S.

### The Variable A

In some embodiments, A is: heteroaryl including from 7-20 ring atoms, wherein from 1-4 ring atoms are heteroatoms, each independently selected from the group consisting of N, N(H), N(R¹), N(R²), O, and S(O)₀₋₂, and wherein from 3-19 ring atoms are carbon atoms, each independently selected from the group consisting of C, CH, CH₂, CR¹, CHR¹, C(R¹)₂, CR³, CHR³, and C(R³)₂.

In certain embodiments, A is: heteroaryl including from 8-12 ring atoms, wherein from 1-4 ring atoms are heteroatoms, each independently selected from the group consisting of N, N(H), N(R¹), N(R²), O, and S(O)₀₋₂, and wherein from 4-11 ring atoms are carbon atoms, each independently selected from the group consisting of C, CH, CH₂, CR¹, CHR¹, C(R¹)₂, CR³, CHR³, and C(R³)₂.

In certain embodiments, A is: heteroaryl including from 8-10 ring atoms, wherein from 1-4 ring atoms are heteroatoms, each independently selected from the group consisting of N, N(H), N(R¹), N(R²), O, and S(O)₀₋₂, and wherein from 4-9 ring atoms are carbon atoms, each independently selected from the group consisting of C, CH, CH₂, CR¹, CHR¹, C(R¹)₂, CR³, CHR³, and C(R³)₂.

In certain embodiments, A is: heteroaryl including from 8-9 ring atoms, wherein from 1-4 ring atoms are heteroatoms, each independently selected from the group consisting of N, N(H), N(R¹), N(R²), O, and S(O)₀₋₂, and wherein from 4-8 ring atoms are carbon atoms, each independently selected from the group consisting of C, CH, CH₂, CR¹, CHR¹, C(R¹)₂, CR³, CHR³, and C(R³)₂.

In certain embodiments, A is (A-1):

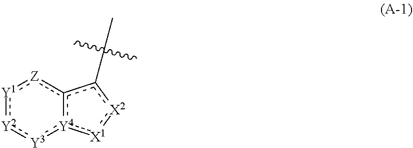

wherein
Z is selected from the group consisting of:
a bond, CH, CR¹, CR³, N, NH, N(R¹) and N(R²);
each of Y¹, Y², and Y³ is independently selected from the group consisting of O, S, CH, CR¹, CR³, N, NH, N(R¹), and NR²;

### Y⁴ is C or N;

X¹ is selected from the group consisting of O, S, N, NH, NR¹, NR², CH, CR¹, and CR³; X² is selected from the group consisting of O, S, N, NH, NR¹, NR², CH, CR¹, and CR³; and
each [symbol] is independently a single bond or a double bond, provided that the five-membered ring comprising Y⁴, X¹, and X² is heteroaryl; and the ring comprising Z, Y¹, Y², Y³, and Y⁴ is aromatic (i.e., carbocyclic aromatic or heteroaromatic).

In some embodiments of (A-1), Z is selected from the group consisting of: CH, CR¹, CR³, N, and N(R²).

In certain embodiments of (A-1), Z is selected from the group consisting of: CH, CR¹, CR³, and N.

In certain embodiments of (A-1), Z is selected from the group consisting of CH, CR¹, and CR³ (e.g., Z is CH).

In some embodiments of (A-1), each of Y¹, Y², and Y³ is independently selected from the group consisting of CH, CR¹, CR³, and N.

In certain embodiments of (A-1), each of Y¹, Y², and Y³ is independently selected from the group consisting of CH, CR¹, and CR³.

In certain embodiments of (A-1), the

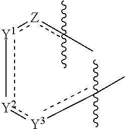

moiety is

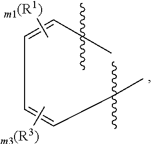

wherein m1=0, 1, 2, or 3; and m3=0, 1, 2, or 3 (e.g., m1=0 or 1; and m3=0, 1, or 2).

In some embodiments of (A-1), from 1-2 of Y¹, Y², and Y³ is independently N.

In certain embodiments of (A-1), one of Y¹, Y², and Y³ is independently N.

In certain of the foregoing embodiments, each of the remaining Y¹, Y², and Y³ is independently selected from the group consisting of CH, CR¹, and CR³.

As a non-limiting example of (A-1), the

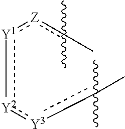

moiety is

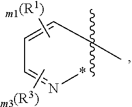

wherein:
the asterisk denotes point of attachment to Y⁴; and
m1=0, 1, or 2; and m3=0, 1, or 2 (e.g., m1=0 or 1; and m3=0 or 1).

In some embodiments of (A-1), Y⁴ is C.

In some embodiments of (A-1), X¹ is selected from the group consisting of O, S, NH, NR¹, and NR².

In certain embodiments of (A-1), X¹ is selected from the group consisting of NH, NR¹, and NR² (e.g., X¹ can be NH).

In some embodiments of (A-1), X² is selected from the group consisting of N, CH, CR¹, and CR³.

In certain embodiments of (A-1), X² is selected from the group consisting of N, C(C₁₋₃ alkyl), and CH.

In certain of these embodiments, X² is CH.

In some embodiments of (A-1), X¹ and X², taken together, is

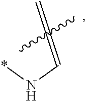 [SMILES: *NC=*]

wherein the asterisk denotes point of attachment to Y⁴.

As a non-limiting example of (A-1), A is:

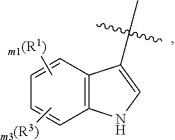

wherein m1=0, 1, 2, or 3; and m3=0, 1, 2, or 3 (e.g., m1=0 or 1; and m3=0, 1, or 2).

As another non-limiting example of (A-1), A is

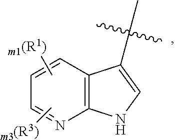

wherein m1=0, 1, or 2; and m3=0, 1, or 2 (e.g., m1=0 or 1; and m3=0 or 1).

As another non-limiting example of (A-1), A is

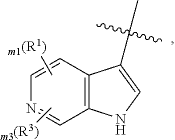

wherein m1=0, 1, or 2; and m3=0, 1, or 2 (e.g., m1=0 or 1; and m3=0 or 1).

As another non-limiting example of (A-1), A is

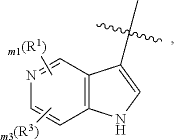

wherein m1=0, 1, or 2; and m3=0, 1, or 2 (e.g., m1=0 or 1; and m3=0 or 1).

As another non-limiting example of (A-1), A is

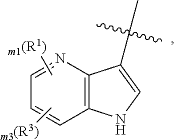

wherein m1=0, 1, or 2; and m3=0, 1, or 2 (e.g., m1=0 or 1; and m3=0 or 1).

In some embodiments, A is (A-2):

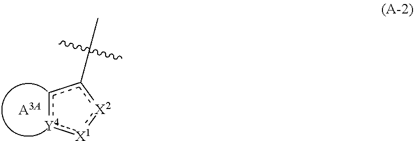

wherein

Ring A^{3A} is a monocyclic or bicyclic ring including from 5-12 ring atoms, wherein from 0-2 ring atoms are heteroatoms (including Y⁴ when Y⁴ is N), wherein each additional heteroatom is independently selected from the group consisting of N, N(H), N(R¹), N(R²), O, and S(O)₀₋₂, and from 3-12 ring atoms are ring carbon atoms each independently selected from C, CH, CH₂, CR¹, CHR¹, C(R¹)₂, CR³, CHR³, and C(R³)₂, provided that Ring A^{3A} is non-aromatic;

X¹ is selected from the group consisting of O, S, N, NH, NR¹, NR², CH, CR¹, and CR³;

X² is selected from the group consisting of O, S, N, NH, NR¹, NR², CH, CR¹, and CR³, provided that the ring including Y⁴, X¹, and X² is heteroaromatic; and

Y⁴ is selected from N or C.

In some embodiments of (A-2), Y⁴ is N.

In some embodiments of (A-2), Ring A^{3A} is a monocyclic or bicyclic ring including from 5-11 ring atoms, wherein from 1-2 ring atoms are heteroatoms (including Y⁴), wherein the additional heteroatom is independently selected from the group consisting of N, N(H), N(R¹), N(R²), O, and S(O)₀₋₂, and from 3-11 ring atoms are ring carbon atoms each independently selected from C, CH, CH₂, CR¹, CHR¹, C(R¹)₂, CR³, CHR³, and C(R³)₂, provided that Ring A^{3A} is non-aromatic.

In certain of these embodiments, Ring A^{3A} is a monocyclic or bicyclic ring including from 5-11 ring atoms, wherein 2 ring atoms are heteroatoms (including Y⁴), wherein the additional heteroatom is independently selected from the group consisting of N, N(H), N(R¹), N(R²), O, and S(O)₀₋₂, and from 3-11 ring atoms are ring carbon atoms each independently selected from C, CH, CH₂, CR¹, CHR¹, C(R¹)₂, CR³, CHR³, and C(R³)₂, provided that Ring A^{3A} is non-aromatic.

In certain embodiments, Ring A^{3A} is a monocyclic or bicyclic ring including from 5-11 ring atoms, wherein 1 ring atom is a heteroatom (including Y⁴), and from 4-11 ring atoms are ring carbon atoms each independently selected from C, CH, CH₂, CR¹, CHR¹, C(R¹)₂, CR³, CHR³, and C(R³)₂, provided that Ring A^{3A} is non-aromatic.

In certain of the foregoing embodiments, Ring A^{3A} is a bicyclic (e.g., spirobicyclic ring) ring contains no additional heteroatoms in addition to Y⁴.

As a non-limiting example of (A-2), A is:

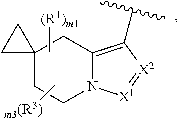

wherein m1=0, 1, or 2; and m3=0, 1, or 2 (e.g., m1=0 or 1; and m3=0 or 1).

In some embodiments of (A-2), Ring A^{3A} is a monocyclic ring that contains an O atom.

As a non-limiting example of (A-2), A is:

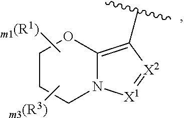

wherein m1=0, 1, or 2; and m3=0, 1, or 2 (e.g., m1=0 or 1; and m3=0 or 1).

In some embodiments of (A-2), X¹ is N.

In some embodiments of (A-2), X² is selected from CH and CR¹ (e.g., CH).

In some embodiments, A is heteroaryl including from 5-6 ring atoms, wherein from 1-4 ring atoms are heteroatoms, each independently selected from the group consisting of N, N(H), N(R¹), N(R²), O, and S, and wherein from 1-5 ring atoms are carbon atoms, each independently selected from the group consisting of C, CH, CR¹, and CR³; provided that at least one ring atom is substituted with R¹.

In certain of these embodiments, A is heteroaryl including 5 ring atoms, wherein from 1-4 ring atoms are heteroatoms, each independently selected from the group consisting of N, N(H), N(R¹), N(R²), O, and S, and wherein from 1-4 ring atoms are carbon atoms, each independently selected from the group consisting of C, CH, CR¹, and CR³; provided that at least one ring atom is substituted with R¹.

In certain of the foregoing embodiments, A is heteroaryl including 5 ring atoms, wherein from 1-4 ring atoms are heteroatoms, each independently selected from the group consisting of N, N(H), N(R¹), N(R²), O, and S, and wherein from 1-4 ring atoms are carbon atoms, each independently selected from the group consisting of C, CH, CR¹, and CR³; provided that one ring atom is substituted with from one R¹.

In certain embodiments, A is (A-3):

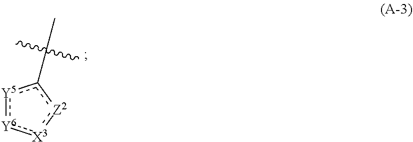

wherein:
Z² is selected from CH, CR², and N;
X³ is selected from O, S, N, NH, NR¹, NR², CH, CR¹, and CR³;
each of Y⁵ and Y⁶ is independently selected from O, S, CH, CR¹, CR³, NR², NH, and N; and
each [symbol] is independently a single bond or a double bond, provided that the five-membered ring comprising Y⁵, Y⁶, X³, and Z² is heteroaromatic.

In some embodiments of (A-3):

when X³ is NR¹ or CR¹, then each of Y⁵ and Y⁶ is independently selected from O, S, CH, CR³, NR², NH, and N; and

when X³ is selected from O, S, N, NH, NR², CH, and CR³, then one of Y⁵ and Y⁶ is CR¹ (in certain embodiments, the other of Y⁵ and Y⁶ is selected from O, S, CH, CR³, NR², NH, and N).

In some embodiments of (A-3), Z² is selected from CH and N.

In certain embodiments of (A-3), Z² is CH.

In some embodiments of (A-3), Y⁶ is selected from N, CH, and CR³.

In certain embodiments of (A-3), Y⁶ is N.

In some embodiments of (A-3), Y⁵ is CR¹.

In some embodiments of (A-3), X³ is selected from S, O, NH, and N(R²) (e.g., NH).

As a non-limiting example of (A-3), A is

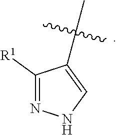

### The Variable R¹

In some embodiments, each occurrence of R¹ is independently selected from:

(i) —(U¹)_{q}—U², wherein:
q is 0 or 1;
U¹ is C₁₋₆ alkylene, which is optionally substituted with from 1-6 R^{a}; and
U² is:

(a) C₃₋₁₀ cycloalkyl, which is optionally substituted with from 1-4 R^{b},

(b) C₆₋₁₀ aryl, which is optionally substituted with from 1-4 R^{c};

(c) heteroaryl including from 5-10 ring atoms, wherein from 1-4 ring atoms are heteroatoms, each independently selected from the group consisting of N, N(H), N(R^{d}), O, S, and S(O)₂ and wherein the heteroaryl ring is optionally substituted with from 1-4 independently selected R^{c}, or

(d) heterocyclyl including from 3-10 ring atoms, wherein from 1-3 ring atoms are heteroatoms, each independently selected from the group consisting of N, N(H), N(R^{d}), and O, and wherein the heterocyclyl ring is optionally substituted with from 1-4 independently selected R^{b},

and

(ii) C₁₋₆ alkyl, which is optionally substituted with from 1-6 independently selected R^{a}.

In certain embodiments, R¹ is —(U¹)_{q}—U².

In certain of these embodiments, q is 0.

In certain embodiments (when R¹ is —(U¹)_{q}—U²), U² is C₆₋₁₀ aryl, which is optionally substituted with from 1-4 R^{c}.

In certain of these embodiments, U² is C₆₋₁₀ aryl, which is optionally substituted with from 1-2 R^{c}.

As a non-limiting example, U² is phenyl, which is optionally substituted with from 1-2 (e.g., 1) R^{c}.

In certain embodiments (when U² is C₆₋₁₀ aryl, which is optionally substituted with from 1-2 R^{c}), each occurrence of R^{c} substituent on U² is independently selected from: halo, cyano, C₁₋₆ alkyl, and C₁₋₄ haloalkyl.

In certain embodiments (when U² is C₆₋₁₀ aryl, which is optionally substituted with from 1-2 R^{c}), each occurrence of R^{c} substituent on U² is independently selected from halo.

In certain embodiments, R¹ is phenyl, which is optionally substituted with from 1-2 (e.g., 0; e.g., 1) R^{c}.

In certain of these embodiments, R^{c} substituent on U² is independently selected from: halo, cyano, C₁₋₆ alkyl, and C₁₋₄ haloalkyl.

In certain embodiments, each occurrence of R^{c} substituent on U² is independently selected from halo (e.g., —F).

### The Variable R³

In some embodiments, each occurrence of R³ is independently selected from the group consisting of: halo, cyano, C₁₋₄ alkoxy, C₁₋₄ haloalkoxy, —S(O)₁₋₂(C₁₋₄ alkyl), —NR^{e}R^{f}, —OH, —S(O)₁₋₂(NR′R″), —C₁₋₄ thioalkoxy, —C(═O)(C₁₋₄ alkyl), —C(═O)O(C₁₋₄ alkyl), —C(═O)OH, and —C(═O)N(R′)(R″).

In certain embodiments, each occurrence of R³ is independently selected from the group consisting of: halo, cyano, C₁₋₄ alkoxy, C₁₋₄ haloalkoxy, —S(O)₁₋₂(C₁₋₄ alkyl), —S(O)₁₋₂(NR′R″), —C(═O)(C₁₋₄ alkyl), —C(═O)O(C₁₋₄ alkyl), —C(═O)OH, and —C(═O)N(R′)(R″).

In certain embodiments, each occurrence of R³ is independently selected from the group consisting of: halo, cyano, C₁₋₄ alkoxy, and C₁₋₄ haloalkoxy (e.g., R³ can be halo).

### The Variable R²

In some embodiments, each occurrence of R² is independently selected from

(i) C₁₋₆ alkyl (e.g., methyl);

(ii) C₃₋₆ cycloalkyl;

(iv) —C(O)(C₁₋₄ alkyl) (e.g., C(O)Me);

(v) —C(O)O(C₁₋₄ alkyl);

(vi) —CON(R′)(R″);

(vii) —S(O)₁₋₂(NR′R″); and

(viii) —S(O)₁₋₂(C₁₋₄ alkyl) (e.g., S(O)₂Me).

Non-Limiting Combinations of A, R¹, and R³

In some embodiments, A is as defined in any one of claims 12, 16, 24, 25, 31, and 33; and m1=1.

In certain of these embodiments, m3=0.

In certain embodiments (when A is as defined in any one of claims 12, 16, 24, 25, 31, and 33; and m1=1), R¹ is as defined in any one of claims 48-57.

In some embodiments, A is as defined in any one of claims 12, 16, 24, 25, 31, and 33; and m1=0.

In certain of these embodiments, m3=0.

In certain other embodiments, m3=1 or 2 (e.g., 1).

In certain embodiments (when A is as defined in any one of claims 12, 16, 24, 25, 31, and 33; m1=0; and m3=1 or 2 (e.g., 1)), each occurrence of R³ is as defined in any one of claims 58-60.

In certain of the foregoing embodiments, each occurrence of R³ is independently halo (e.g., F).

### The Variable B

In some embodiments, B is phenyl substituted with from 1-4 R^{c}.

In certain embodiments, B is phenyl substituted with from 1-2 R^{c}, wherein one R^{c} is at the ring carbon para to the point of attachment to the L^{AB} moiety in Formula I.

In certain embodiments, B is phenyl substituted with one R^{c} which is at the ring carbon para to the point of attachment to the L^{AB} moiety in Formula I (i.e.,

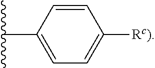

In some embodiments (e.g., when B is phenyl optionally substituted with from 1-4 R^{c}), each occurrence of R^{c} substituent on B is independently selected from:
(a) halo;
(b) cyano;
(c) C₁₋₁₀ alkyl which is optionally substituted with from 1-6 independently selected R^{a};
(g) C₁₋₄ alkoxy;
(h) C₁₋₄ haloalkoxy;
(i) —S(O)₁₋₂(C₁₋₄ alkyl);
(m) —C₁₋₄ thioalkoxy;
(o) —C(═O)(C₁₋₄ alkyl);
(p) —C(═O)O(C₁₋₄ alkyl);
(r) —C(═O)N(R′)(R″); and
(s) -L¹-L²-R^{h}.

In certain embodiments, each occurrence of R^{c} substituent on B is independently selected from:
(a) halo;
(b) cyano;
(c) C₁₋₁₀ alkyl which is optionally substituted with from 1-6 independently selected R^{a};
(g) C₁₋₄ alkoxy;
(h) C₁₋₄ haloalkoxy; and
(s) -L¹-L²-R^{h}.

In certain embodiments, each occurrence of R^{c} substituent on B is independently selected from:
(a) halo;
(b) cyano;
(c) C₁₋₁₀ alkyl which is optionally substituted with from 1-6 independently selected R^{a};
(g) C₁₋₄ alkoxy optionally substituted with from 1-2 independently selected R^{a}; (h) C₁₋₄ haloalkoxy; and
(s) -L¹-L²-R^{h}.

In certain embodiments, each occurrence of R^{c} substituent on B is independently selected from:
(a) halo;
(c) C₁₋₁₀ alkyl which is optionally substituted with from 1-6 independently selected R^{a}; and
(s) -L¹-L²-R^{h}.

In certain embodiments, one occurrence of R^{c} is C₁₋₁₀ alkyl which is optionally substituted with from 1-6 independently selected R^{a}.

In certain embodiments, one occurrence of R^{c} is C₁₋₆ alkyl which is optionally substituted with from 1-6 independently selected R^{a}.

In certain of these embodiments, one occurrence of R^{c} is unsubstituted C₁₋₁₀ alkyl.

As a non-limiting example, one occurrence of R^{c} is unsubstituted C₂₋₁₀ (e.g., C₂₋₃, e.g., C₃₋₄, e.g., C₄₋₁₀) alkyl.

In certain embodiments, one occurrence of R^{c} is C₁₋₆ alkyl which is substituted with from 1-6 independently selected R^{a}.

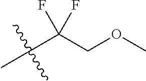 [SMILES: *C(F)(F)COC]

As a non-limiting example, one occurrence of R^{c} is CF₃ or (e.g., R^{c} can be CF₃).

In any of the foregoing embodiments (e.g., when one occurrence of R^{c} is C₁₋₁₀ alkyl which is optionally substituted with from 1-6 independently selected R^{a}), a second occurrence of R^{c} when present is independently halo.

In any of the foregoing embodiments (e.g., when one occurrence of R^{c} is C₁₋₁₀ alkyl which is optionally substituted with from 1-6 independently selected R^{a}), B is phenyl substituted with from 1-3 occurrences of R^{c}; and one occurrence of R^{c} is at the ring carbon para to the point of attachment to the L^{AB} moiety in Formula I.

In certain embodiments, B is selected from the group consisting of:

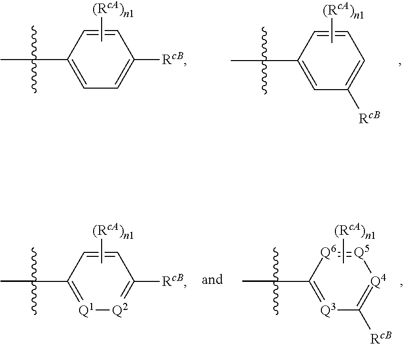

wherein each R^{cA} and R^{cB} is an independently selected R^{c}; n1 is 0, 1, or 2; each of Q¹, Q², Q³, Q⁴, Q⁵, and Q⁶ is independently selected from the group consisting of N and CH, provided that at least one of Q¹ and Q² is N; and at least one of Q³, Q⁴, Q⁵, and Q⁶ is N.

In certain of these embodiments, n1 is 0.

In certain other embodiments, n1 is 1. In certain of these embodiments, R^{cA} is halo (e.g., —F, or —Cl) or C₁₋₆ alkyl which is optionally substituted with from 1-3 independently selected R^{a} (e.g., methyl or CF₃).

In certain embodiments, R^{cB} is C₁₋₆ alkyl which is optionally substituted with from 1-6 independently selected R^{a}.

For example, R^{cB} can be unsubstituted C₂₋₁₀ (e.g., C₂₋₃, e.g., C₃₋₄, e.g., C₄₋₁₀) alkyl.

As another non-limiting example, R^{cB} can be C₁₋₆ alkyl which is substituted with from 1-6 independently selected R^{a}. For example, each R^{a} can be halo (e.g., F), NR^{e}R^{f}, OH, C₁₋₃ alkoxy, or C₁₋₃ haloalkoxy.

In certain embodiments, R^{cB} is -L¹-L²-R^{h}. In certain of these embodiments, each of L¹ and L² is a bond. In certain other embodiments, L¹ is a bond; and L² is —O—.

In certain embodiments, R^{h} is selected from the group consisting of:

C₃₋₆ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);

heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 substituents independently selected from the group consisting of halo, C₁₋₄ alkyl, and C₁₋₄ haloalkyl; and

C₆ aryl, which is optionally substituted with from 1-4 substituents independently selected from the group consisting of halo, C₁₋₄ alkyl, or C₁₋₄ haloalkyl.

In certain embodiments, B is heteroaryl including 5 ring atoms, wherein from 1-3 ring atoms are heteroatoms, each independently selected from the group consisting of N, N(H), N(R^{d}), O, and S(O)₀₋₂, and wherein the heteroaryl ring is substituted with from 1-4 independently selected R^{c}, provided that one occurrence of R^{c} is L¹-L²-R^{h}. In certain of these embodiments, each of L¹ and L² is a bond. In certain other embodiments, L¹ is a bond; and L² is —O—.

In certain embodiments, R^{h} is selected from the group consisting of:

C₃₋₆ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);

heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 substituents independently selected from the group consisting of halo, C₁₋₄ alkyl, and C₁₋₄ haloalkyl; and

C₆ aryl, which is optionally substituted with from 1-4 substituents independently selected from the group consisting of halo, C₁₋₄ alkyl, or C₁₋₄ haloalkyl.

In certain embodiments, B is

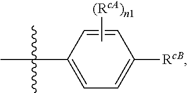

wherein:
n1=0 or 1; and
each of R^{cA} and R^{cB} is an independently selected R^{c}.

In certain of these embodiments, R^{cB} is R^{c} that is as defined in any one of claims 76-82.

In certain of the foregoing embodiments, R^{cB} is R^{c} that is as defined in any one of claims 78-79.

In certain embodiments, R^{cB} is unsubstituted C₁₋₁₀ alkyl.

In certain embodiments, R^{cB} is unsubstituted C₂₋₁₀ (e.g., C₂₋₃, e.g., C₃₋₄, e.g., C₄-10) alkyl.

In certain of the foregoing embodiments, R^{cB} is R^{c} that is as defined in any one of claims 80-81.

In certain embodiments, R^{cB} is C₁₋₆ alkyl which is substituted with from 1-6 independently selected R^{a}.

In certain embodiments, R^{cB} is is CF₃ or

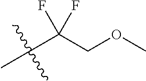 [SMILES: *C(F)(F)COC]

(e.g., R^{c} can be CF₃).

In certain embodiments (when B is

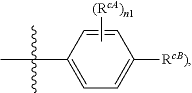

n1 is 0.

In certain embodiments (when B is

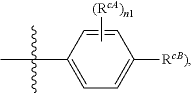

n1 is 1; and R^{cA} is halo.

In some embodiments, B is C₅₋₁₅ (e.g., C₅₋₇, C₈₋₁₀, C₁₁₋₁₃, or C₁₄₋₁₅) alkyl which is optionally substituted with from 1-6 R^{a}.

In certain embodiments, B is C₅₋₁₅ (e.g., C₅₋₇, C₈₋₁₀, C₁₁₋₁₃, or C₁₄₋₁₅) alkyl which is optionally substituted with from 1-3 R^{a}.

In certain embodiments, B is C₅₋₁₅ (e.g., C₅₋₇, C₈₋₁₀, C₁₁₋₁₃, or C₁₄₋₁₅) alkyl. For example, B can be C₈, C₉, C₁₀, C₁₁, C₁₂, C₁₃, C₁₄, or C₁₅ alkyl (e.g., straight-chain alkyl).

The Variable L^{AB}In some embodiments, L^{AB} is —N(R^{N})S(O)₂—*.

In some embodiments, L^{AB} is —S(O)₂N(R^{N})—*.

In some embodiments, L^{AB} is —N(R^{N})S(O)₂—(W^{AB1}—W^{AB2}—W^{AB3})—*.

In certain of these embodiments, W^{AB1} is C₁₋₃ alkylene.

In certain of the foregoing embodiments, W^{AB2} is a bond. In certain other embodiments, W^{AB2} is —O— or —S— (e.g., —O—).

In certain embodiments, W^{AB3} is a bond. In certain other embodiments, W^{AB3} is C₁₋₃ alkylene.

As non-limiting examples when L^{AB} is —N(R^{N})S(O)₂—(W^{AB1}—W^{AB2}—W^{AB3})—*, L^{AB} can be: CH₂, CH₂CH₂, CH₂CH₂CH₂, or CH₂CH₂CH₂OCH₂*.

In some embodiments, R^{N} is H.

### Non-Limiting Combinations

### [I-1]

In some embodiments, the compound has Formula (I-1):

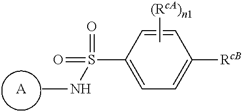

wherein n1=0 or 1; and
each of R^{cA} and R^{cB} is an independently selected R^{c}.

### [I-2]

In some embodiments, the compound has Formula (I-2):

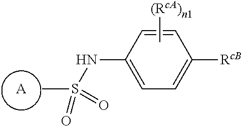

wherein n1=0 or 1; and
each of R^{cA} and R^{cB} is an independently selected R^{c}.

In some embodiments of [I-1] and [I-2], A is (A-1) as defined in claim 6.

In certain embodiments of [I-1] and [I-2], A is as defined in claim 24.

In certain embodiments of [I-1] and [I-2], A is as defined in claim 25.

In certain embodiments of [I-1] and [I-2] (when A is as defined in claim 24; or when A is as defined in claim 25), m1=0.

In certain of these embodiments, m3=1.

In certain of the foregoing embodiments, R³ is as defined in any one of claims 48-50.

In certain embodiments of [I-1] and [I-2] (when A is as defined in claim 24, or when A is as defined in claim 25; and m1=0), m3=0.

In some embodiments of [I-1] and [I-2], A is (A-2) as defined in claim 26.

In certain embodiments of [I-1] and [I-2], A is as defined in any one of claims 30-31 (e.g., claim 31).

In certain embodiments of [I-1] and [I-2], A is as defined in any one of claims 32-33 (e.g., claim 33).

In certain embodiments of [I-1] and [I-2] (when A is as defined in any one of claims 30-31 (e.g., claim 31); or when A is as defined in any one of claims 32-33 (e.g., claim 33)), m1=0.

In certain of these embodiments, m3=0.

In some embodiments of [I-1] and [I-2], A is (A-3) as defined in claim 39.

In certain of the foregoing embodiments, A is as defined in claim 47.

In certain embodiments of [I-1] and [I-2] (when A is (A-3) as defined in claim 39), R¹ is as defined in any one of claims 56-57.

In some embodiments of [I-1] and [I-2], R^{cB} is R^{c} that is as defined in any one of claims 76-82.

In some embodiments of [I-1] and [I-2], R^{cB} is R^{c} that is as defined in any one of claims 78-79.

In some embodiments of [I-1] and [I-2], R^{cB} is R^{c} that is as defined in any one of claims 80-81.

In some embodiments of [I-1] and [I-2], n1 is 0.

In some embodiments of [I-1] and [I-2], n1 is 1; and R^{cA} is halo.

### [I-3]

In some embodiments, A is selected from the group consisting of:

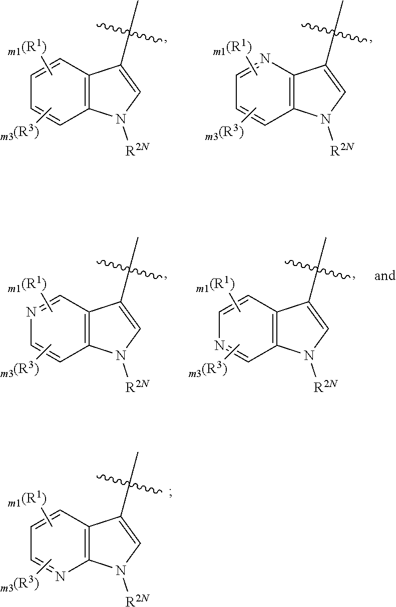

R^{2N} is H or R²;

m1 is 0 or 1; and m3 is 0, 1, or 2;

L^{AB} is —N(H)S(O)₂—* and —NHS(O)₂—(W^{AB1})—*; and

B is selected from the group consisting of:

C aryl substituted with from 1-4 R^{c};

heteroaryl including from 5-6 ring atoms, wherein from 1-3 ring atoms are heteroatoms, each independently selected from the group consisting of N, N(H), N(R^{d}), O, and S(O)₀₋₂, and wherein the heteroaryl ring is substituted with from 1-4 independently selected R^{c};

bicyclic or tricyclic heteroaryl including from 9-15 ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c};

C₅₋₁₅ alkyl which is optionally substituted with from 1-6 R^{a}; and

C₈₋₂₀ aryl optionally substituted with from 1-4 R^{c}.

In certain embodiments of [I-3], R^{2N} is H.

In certain embodiments of [I-3], m1 is 0.

In certain other embodiments of [1-3], m1 is 1.

In certain embodiments of [I-3], m3 is 0.

In certain other embodiments, m3 is 1 or 2.

In certain embodiments of [1-3], m1 is 0; and m3 is 1 or 2 (e.g., 2).

In certain embodiments of [1-3], A is

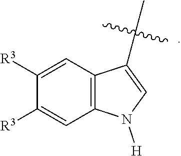

For example, each R³ can be halo (e.g., F).

In certain embodiments of [1-3], each occurrence of R³ is independently selected from the group consisting of: halo, cyano, C₁₋₄ alkoxy, C₁₋₄ haloalkoxy, —S(O)₁₋₂(C₁₋₄ alkyl), —S(O)₁₋₂(NR′R″), —C(═O)(C₁₋₄ alkyl), —C(═O)O(C₁₋₄ alkyl), —C(═O)OH, and —C(═O)N(R′)(R″).

In certain embodiments of [1-3], each occurrence of R³ is independently selected from the group consisting of: halo, cyano, C₁₋₄ alkoxy, and C₁₋₄ haloalkoxy (e.g., R³ can be halo).

In certain embodiments of [1-3], R¹ is —(U¹)_{q}—U².

In certain of these embodiments, q is 0.

In certain embodiments of [1-3] (when R¹ is —(U¹)_{q}—U²), U² is C₆₋₁₀ aryl, which is optionally substituted with from 1-4 R^{c}.

In certain of these embodiments of [1-3], U² is C₆₋₁₀ aryl, which is optionally substituted with from 1-2 R^{c}.

As a non-limiting example, U² is phenyl, which is optionally substituted with from 1-2 (e.g., 1) R^{c}.

In certain embodiments of [1-3] (when U² is C₆₋₁₀ aryl, which is optionally substituted with from 1-2 R^{c}), each occurrence of R^{c} substituent on U² is independently selected from: halo, cyano, C₁₋₆ alkyl, and C₁₋₄ haloalkyl.

In certain embodiments of [1-3] (when U² is C₆₋₁₀ aryl, which is optionally substituted with from 1-2 R^{c}), each occurrence of R^{c} substituent on U² is independently selected from halo.

In certain embodiments of [1-3], L^{AB} is NHS(O)₂—*. In certain other embodiments, L^{AB} is NHS(O)₂—(C₁₋₃ alkylene)-*.

In certain embodiments of [1-3], B is selected from the group consisting of:

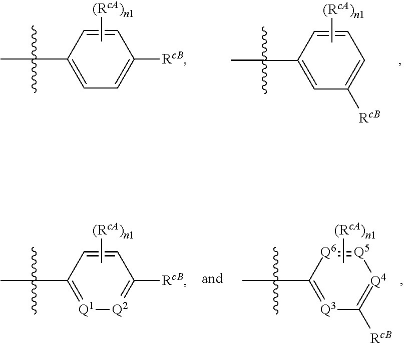

wherein each R^{cA} and R^{cB} is an independently selected R^{c}; n1 is 0, 1, or 2; each of Q¹, Q², Q³, Q⁴, Q⁵, and Q⁶ is independently selected from the group consisting of N and CH, provided that at least one of Q¹ and Q² is N; and at least one of Q³, Q⁴, Q⁵, and Q⁶ is N.

In certain of these embodiments, n1 is 0.

In certain other embodiments, n1 is 1. In certain of these embodiments, R^{cA} is halo (e.g., —F, or —Cl) or C₁₋₆ alkyl which is optionally substituted with from 1-3 independently selected R^{a} (e.g., methyl or CF₃).

In certain embodiments, R^{cB} is C₁₋₆ alkyl which is optionally substituted with from 1-6 independently selected R^{a}.

For example, R^{cB} can be unsubstituted C₂₋₁₀ (e.g., C₂₋₃, e.g., C₃₋₄, e.g., C₄₋₁₀) alkyl.

As another non-limiting example, R^{cB} can be C₁₋₆ alkyl which is substituted with from 1-6 independently selected R^{a}. For example, each R^{a} can be halo (e.g., F), NR^{e}R^{f}, OH, C₁₋₃ alkoxy, or C₁₋₃ haloalkoxy.

In certain embodiments, R^{cB} is -L¹-L²-R^{h}. In certain of these embodiments, each of L¹ and L² is a bond. In certain other embodiments, L¹ is a bond; and L² is —O—.

In certain embodiments, R^{h} is selected from the group consisting of:

C₃₋₆ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);

heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 substituents independently selected from the group consisting of halo, C₁₋₄ alkyl, and C₁₋₄ haloalkyl; and

C₆ aryl, which is optionally substituted with from 1-4 substituents independently selected from the group consisting of halo, C₁₋₄ alkyl, or C₁₋₄ haloalkyl.

In certain embodiments of [1-3], B is heteroaryl including 5 ring atoms, wherein from 1-3 ring atoms are heteroatoms, each independently selected from the group consisting of N, N(H), N(R^{d}), O, and S(O)₀₋₂, and wherein the heteroaryl ring is substituted with from 1-4 independently selected R^{c}, provided that one occurrence of R^{c} is L¹-L²-R^{h}. In certain of these embodiments, each of L¹ and L² is a bond. In certain other embodiments, L¹ is a bond; and L² is —O—.

In certain embodiments, R^{h} is selected from the group consisting of:

C₃₋₆ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);

heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 substituents independently selected from the group consisting of halo, C₁₋₄ alkyl, and C₁₋₄ haloalkyl; and

C₆ aryl, which is optionally substituted with from 1-4 substituents independently selected from the group consisting of halo, C₁₋₄ alkyl, or C₁₋₄ haloalkyl.

### Non-Limiting Exemplary Compounds

In some embodiments, the compound is selected from the following:

TABLE C1

| Compound | Structure |
|---|---|
| 101 | 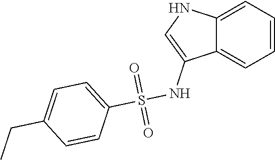 [SMILES: CCc1ccc(S(=O)(=O)Nc2c[nH]c3ccccc23)cc1] |
| 102 | 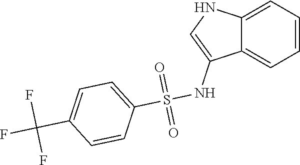 [SMILES: O=S(=O)(Nc1c[nH]c2ccccc12)c1ccc(C(F)(F)F)cc1] |
| 103 | 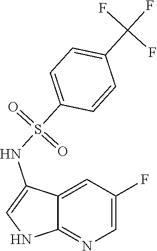 [SMILES: O=S(=O)(Nc1c[nH]c2ncc(F)cc12)c1ccc(C(F)(F)F)cc1] |
| 104 | 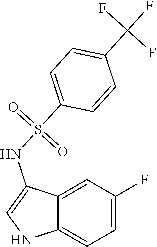 [SMILES: O=S(=O)(Nc1c[nH]c2ccc(F)cc12)c1ccc(C(F)(F)F)cc1] |
| 105 | 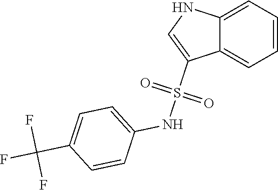 [SMILES: O=S(=O)(Nc1ccc(C(F)(F)F)cc1)c1c[nH]c2ccccc12] |
| 106 | 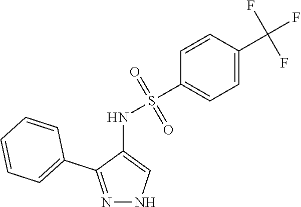 |
| 107 | 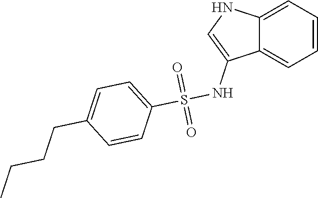 [SMILES: CCCCc1ccc(S(=O)(=O)Nc2c[nH]c3ccccc23)cc1] |
| 108 | 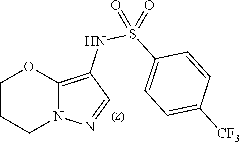 [SMILES: O=S(=O)(Nc1cnn2c1OCCC2)c1ccc(C(F)(F)F)cc1] |
| 109 | 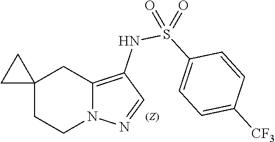 [SMILES: O=S(=O)(Nc1cnn2c1CC1(CC2)CC1)c1ccc(C(F)(F)F)cc1] |
| 110 | 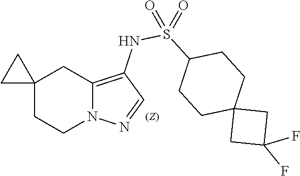 [SMILES: O=S(=O)(Nc1cnn2c1CC1(CC2)CC1)C1CCC2(CC1)CC(F)(F)C2] |
| 111 | 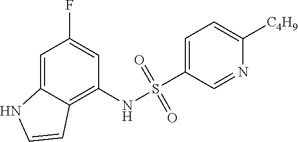 |
| 112 | 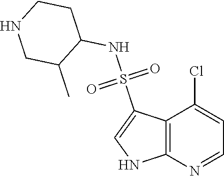 [SMILES: CC1CNCCC1NS(=O)(=O)c1c[nH]c2nccc(Cl)c12] |
| 113 | 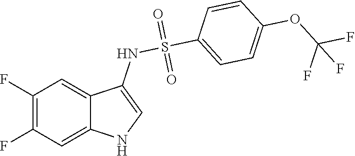 [SMILES: O=S(=O)(Nc1c[nH]c2cc(F)c(F)cc12)c1ccc(OC(F)(F)F)cc1] |
| 114 | 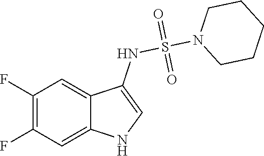 [SMILES: O=S(=O)(Nc1c[nH]c2cc(F)c(F)cc12)N1CCCCC1] |
| 115 | 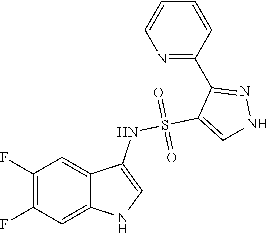 [SMILES: O=S(=O)(Nc1c[nH]c2cc(F)c(F)cc12)c1c[nH]nc1-c1ccccn1] |
| 116 | 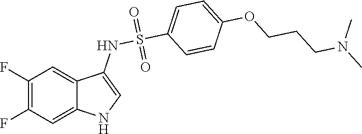 [SMILES: CN(C)CCCOc1ccc(S(=O)(=O)Nc2c[nH]c3cc(F)c(F)cc23)cc1] |
| 117 | 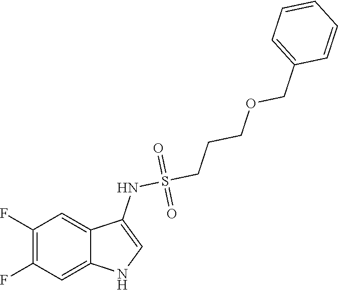 [SMILES: O=S(=O)(CCCOCc1ccccc1)Nc1c[nH]c2cc(F)c(F)cc12] |
| 118 | 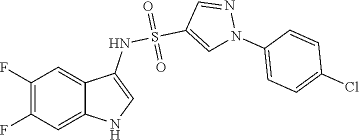 [SMILES: O=S(=O)(Nc1c[nH]c2cc(F)c(F)cc12)c1cnn(-c2ccc(Cl)cc2)c1] |
| 119 | 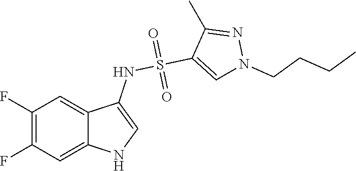 [SMILES: CCCCn1cc(S(=O)(=O)Nc2c[nH]c3cc(F)c(F)cc23)c(C)n1] |
| 120 | 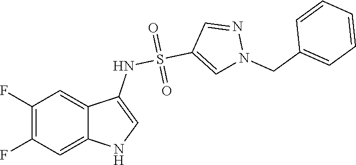 [SMILES: O=S(=O)(Nc1c[nH]c2cc(F)c(F)cc12)c1cnn(Cc2ccccc2)c1] |
| 121 | 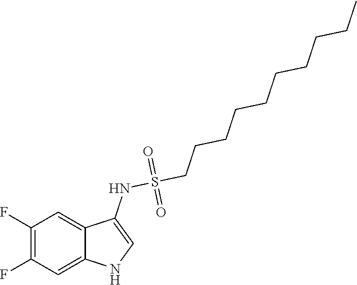 [SMILES: CCCCCCCCCCS(=O)(=O)Nc1c[nH]c2cc(F)c(F)cc12] |
| 122 | 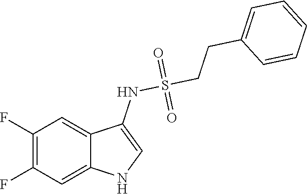 [SMILES: O=S(=O)(CCc1ccccc1)Nc1c[nH]c2cc(F)c(F)cc12] |
| 123 | 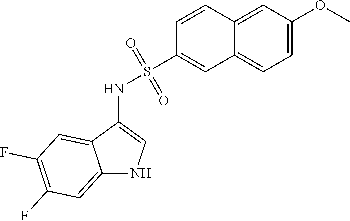 |
| 124 | 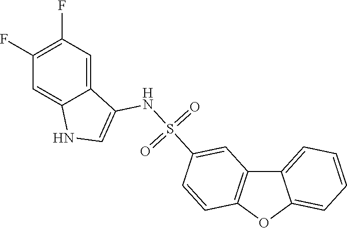 [SMILES: O=S(=O)(Nc1c[nH]c2cc(F)c(F)cc12)c1ccc2oc3ccccc3c2c1] |
| 125 | 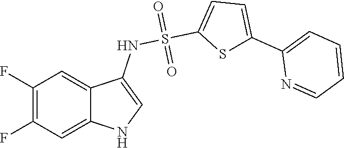 [SMILES: O=S(=O)(Nc1c[nH]c2cc(F)c(F)cc12)c1ccc(-c2ccccn2)s1] |
| 126 | 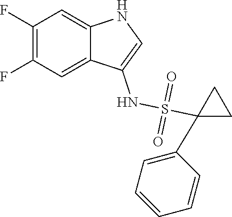 [SMILES: O=S(=O)(Nc1c[nH]c2cc(F)c(F)cc12)C1(c2ccccc2)CC1] |
| 127 | 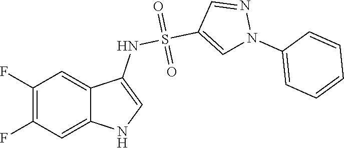 [SMILES: O=S(=O)(Nc1c[nH]c2cc(F)c(F)cc12)c1cnn(-c2ccccc2)c1] |
| 128 | 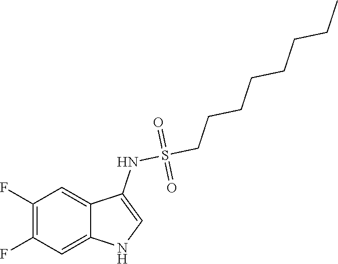 [SMILES: CCCCCCCCS(=O)(=O)Nc1c[nH]c2cc(F)c(F)cc12] |
| 129 | 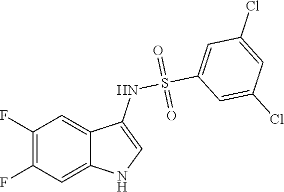 [SMILES: O=S(=O)(Nc1c[nH]c2cc(F)c(F)cc12)c1cc(Cl)cc(Cl)c1] |
| 130 | 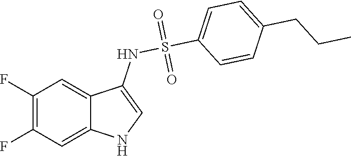 [SMILES: CCCc1ccc(S(=O)(=O)Nc2c[nH]c3cc(F)c(F)cc23)cc1] |
| 131 | 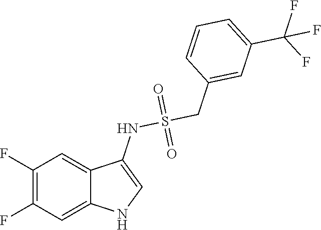 [SMILES: O=S(=O)(Cc1cccc(C(F)(F)F)c1)Nc1c[nH]c2cc(F)c(F)cc12] |
| 132 | 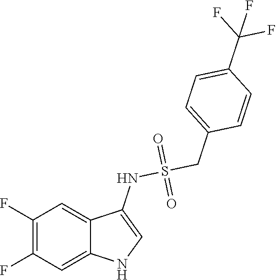 [SMILES: O=S(=O)(Cc1ccc(C(F)(F)F)cc1)Nc1c[nH]c2cc(F)c(F)cc12] |
| 133 | 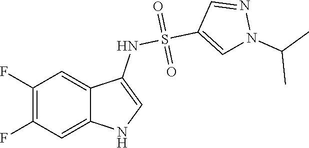 [SMILES: CC(C)n1cc(S(=O)(=O)Nc2c[nH]c3cc(F)c(F)cc23)cn1] |
| 134 | 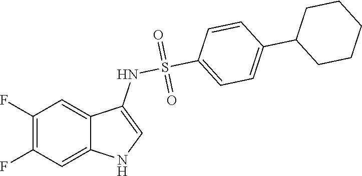 [SMILES: O=S(=O)(Nc1c[nH]c2cc(F)c(F)cc12)c1ccc(C2CCCCC2)cc1] |
| 135 | 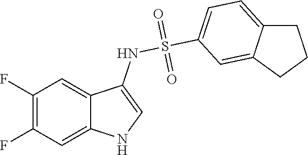 [SMILES: O=S(=O)(Nc1c[nH]c2cc(F)c(F)cc12)c1ccc2c(c1)CCC2] |
| 136 | 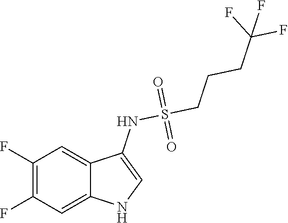 [SMILES: O=S(=O)(CCCC(F)(F)F)Nc1c[nH]c2cc(F)c(F)cc12] |
| 137 | 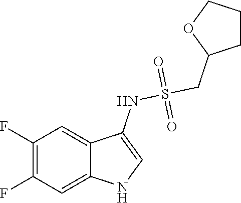 [SMILES: O=S(=O)(CC1CCCO1)Nc1c[nH]c2cc(F)c(F)cc12] |
| 138 | 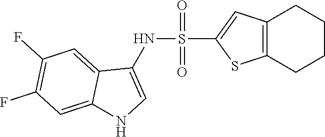 [SMILES: O=S(=O)(Nc1c[nH]c2cc(F)c(F)cc12)c1cc2c(s1)CCCC2] |
| 139 | 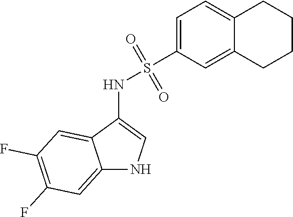 |
| 140 | 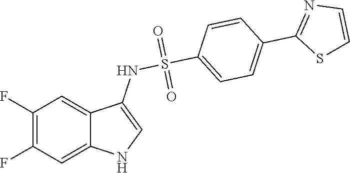 [SMILES: O=S(=O)(Nc1c[nH]c2cc(F)c(F)cc12)c1ccc(-c2nccs2)cc1] |
| 141 | 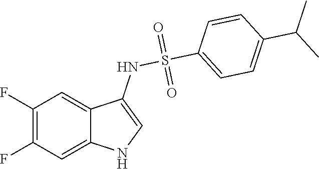 [SMILES: CC(C)c1ccc(S(=O)(=O)Nc2c[nH]c3cc(F)c(F)cc23)cc1] |
| 142 | 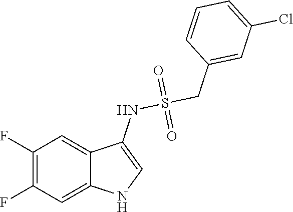 [SMILES: O=S(=O)(Cc1cccc(Cl)c1)Nc1c[nH]c2cc(F)c(F)cc12] |
| 143 | 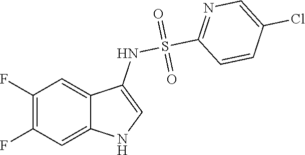 [SMILES: O=S(=O)(Nc1c[nH]c2cc(F)c(F)cc12)c1ccc(Cl)cn1] |
| 144 | 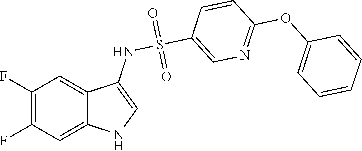 [SMILES: O=S(=O)(Nc1c[nH]c2cc(F)c(F)cc12)c1ccc(Oc2ccccc2)nc1] |
| 145 | 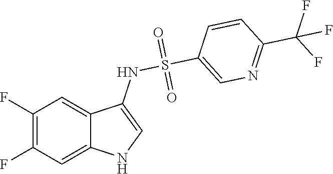 [SMILES: O=S(=O)(Nc1c[nH]c2cc(F)c(F)cc12)c1ccc(C(F)(F)F)nc1] |
| 146 | 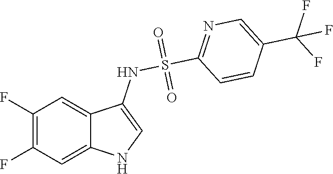 [SMILES: O=S(=O)(Nc1c[nH]c2cc(F)c(F)cc12)c1ccc(C(F)(F)F)cn1] |
| 147 | 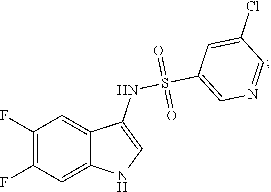 [SMILES: O=S(=O)(Nc1c[nH]c2cc(F)c(F)cc12)c1cncc(Cl)c1] |

or a pharmaceutically acceptable salt thereof.

This specification concludes with 170 claims. For ease of exposition, certain variable definitions refer to one or more specific claim numbers, and as such, it is understood that the entire subject matter of each claim referenced is incorporated by reference in its entirety into the portion of the disclosure, in which it is referenced. For the avoidance of doubt and as a non-limiting example, use of a phrase, such as “A is as defined in claim 24” is intended to represent a short-hand recitation for the following set of definition:

A is:

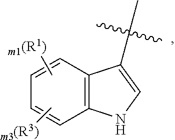

wherein m1=0, 1, 2, or 3; and m3=0, 1, 2, or 3 (e.g., m1=0 or 1; and m3=0, 1, or 2).

Pharmaceutical Compositions and Administration

General

In some embodiments, a chemical entity (e.g., a compound that inhibits (e.g., antagonizes) STING, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 1 1 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 1 1 months, 12 months, or more. In a further embodiment, a period of during which administration is stopped is for 1 day, 2 days, 3 days, 4 days, 5 days, 6 days, 7 days, 8 days, 9 days, 10 days, 1 1 days, 12 days, 13 days, 14 days, 3 weeks, 4 weeks, 5 weeks, 6 weeks, 7 weeks, 8 weeks, 9 weeks, 10 weeks, 1 1 weeks, 12 weeks, 4 months, 5 months, 6 months, 7 months, 8 months, 9 months, 10 months, 1 1 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Methods of Treatment

In some embodiments, methods for treating a subject having condition, disease or disorder in which increased (e.g., excessive)STING activity (e.g., e.g., STING signaling) contributes to the pathology and/or symptoms and/or progression of the condition, disease or disorder (e.g., immune disorders, cancer) are provided.

Indications

In some embodiments, the condition, disease or disorder is cancer. Non-limiting examples of cancer include melanoma, carcinoma, lymphoma, blastoma, sarcoma, and leukemia or lymphoid malignancies. More particular examples of such cancers include breast cancer, colon cancer, rectal cancer, colorectal cancer, kidney or renal cancer, clear cell cancer lung cancer including small-cell lung cancer, non-small cell lung cancer, adenocarcinoma of the lung and squamous carcinoma of the lung, squamous cell cancer (e.g. epithelial squamous cell cancer), cervical cancer, ovarian cancer, prostate cancer, prostatic neoplasms, liver cancer, bladder cancer, cancer of the peritoneum, hepatocellular cancer, gastric or stomach cancer including gastrointestinal cancer, gastrointestinal stromal tumor, pancreatic cancer, head and neck cancer, glioblastoma, retinoblastoma, astrocytoma, thecomas, arrhenoblastomas, hepatoma, hematologic malignancies including non-Hodgkins lymphoma (NHL), multiple myeloma, myelodysplasia disorders, myeloproliferative disorders, chronic myelogenous leukemia, and acute hematologic malignancies, endometrial or uterine carcinoma, endometriosis, endometrial stromal sarcoma, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, mast cell sarcoma, ovarian sarcoma, uterine sarcoma, melanoma, malignant mesothelioma, skin carcinomas, Schwannoma, oligodendroglioma, neuroblastomas, neuroectodermal tumor, rhabdomyosarcoma, osteogenic sarcoma, leiomyosarcomas, Ewing Sarcoma, peripheral primitive neuroectodermal tumor, urinary tract carcinomas, thyroid carcinomas, Wilm's tumor, as well as abnormal vascular proliferation associated with phakomatoses, edema (such as that associated with brain tumors), and Meigs' syndrome. In some cases, the cancer is melanoma.

In some embodiments, the condition, disease or disorder is a neurological disorder, which includes disorders that involve the central nervous system (brain, brainstem and cerebellum), the peripheral nervous system (including cranial nerves), and the autonomic nervous system (parts of which are located in both central and peripheral nervous system). Non-limiting examples of cancer include acquired epileptiform aphasia; acute disseminated encephalomyelitis; adrenoleukodystrophy; age-related macular degeneration; agenesis of the corpus callosum; agnosia; Aicardi syndrome; Alexander disease; Alpers' disease; alternating hemiplegia; Alzheimer's disease; Vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telegiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome; causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; Dandy-Walker syndrome; Dawson disease; De Morsier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia and other “tauopathies”; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile phytanic acid storage disease; infantile refsum disease; infantile spasms; inflammatory myopathy; intracranial cyst; intracranial hypertension; Joubert syndrome; Kearns-Sayre syndrome; Kennedy disease Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly; locked-in syndrome; Lou Gehrig's disease (i.e., motor neuron disease or amyotrophic lateral sclerosis); lumbar disc disease; Lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; milti-infarct dementia; multifocal motor neuropathy; multiple sclerosis and other demyelinating disorders; multiple system atrophy with postural hypotension; p muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocal leukoencephalopathy; progressive sclerosing poliodystrophy; progressive supranuclear palsy; pseudotumor cerebri; Ramsay-Hunt syndrome (types I and II); Rasmussen's encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia; shaken baby syndrome; shingles; Shy-Drager syndrome; Sjögren's syndrome; sleep apnea; Soto's syndrome; spasticity; spina bifida; spinal cord injury; spinal cord tumors; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau disease; Wallenberg's syndrome; Werdnig-Hoffman disease; West syndrome; whiplash; Williams syndrome; Wildon's disease; amyotrophe lateral sclerosis and Zellweger syndrome.

In some embodiments, the condition, disease or disorder is STING-associated conditions, e.g., type I interferonopathies (e.g., STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis. In certain embodiments, the condition, disease or disorder is an autoimmune disease (e.g., a cytosolic DNA-triggered autoinflammatory disease). Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, modulation of the immune system by STING provides for the treatment of diseases, including diseases caused by foreign agents. Exemplary infections by foreign agents which may be treated and/or prevented by the method of the present invention include an infection by a bacterium (e.g., a Gram-positive or Gram-negative bacterium), an infection by a fungus, an infection by a parasite, and an infection by a virus. In one embodiment of the present invention, the infection is a bacterial infection (e.g., infection by E. coli, Klebsiella pneumoniae, Pseudomonas aeruginosa, Salmonella spp., Staphylococcus aureus, Streptococcus spp., or vancomycin-resistant enterococcus), or sepsis. In another embodiment, the infection is a fungal infection (e.g. infection by a mould, a yeast, or a higher fungus). In still another embodiment, the infection is a parasitic infection (e.g., infection by a single-celled or multicellular parasite, including Giardia duodenalis, Cryptosporidium parvum, Cyclospora cayetanensis, and Toxoplasma gondiz). In yet another embodiment, the infection is a viral infection (e.g., infection by a virus associated with AIDS, avian flu, chickenpox, cold sores, common cold, gastroenteritis, glandular fever, influenza, measles, mumps, pharyngitis, pneumonia, rubella, SARS, and lower or upper respiratory tract infection (e.g., respiratory syncytial virus)).

In some embodiments, the condition, disease or disorder is hepatitis B (see, e.g., WO 2015/061294).

In some embodiments, the condition, disease or disorder is selected from cardiovascular diseases (including e.g., myocardial infarction).

In some embodiments, the condition, disease or disorder is age-related macular degeneration.

In some embodiments, the condition, disease or disorder is mucositis, also known as stomatitits, which can occur as a result of chemotherapy or radiation therapy, either alone or in combination as well as damage caused by exposure to radiation outside of the context of radiation therapy.

In some embodiments, the condition, disease or disorder is uveitis, which is inflammation of the uvea (e.g., anterior uveitis, e.g., iridocyclitis or iritis; intermediate uveitis (also known as pars planitis); posterior uveitis; or chorioretinitis, e.g., pan-uveitis).

In some embodiments, the condition, disease or disorder is selected from the group consisting of a cancer, a neurological disorder, an autoimmune disease, hepatitis B, uvetitis, a cardiovascular disease, age-related macular degeneration, and mucositis.

Still other examples can include those indications discussed herein and below in contemplated combination therapy regimens.

Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the methods described herein can further include administering one or more additional cancer therapies.

The one or more additional cancer therapies can include, without limitation, surgery, radiotherapy, chemotherapy, toxin therapy, immunotherapy, cryotherapy, cancer vaccines (e.g., HIPV vaccine, hepatitis B vaccine, Oncophage, Provenge) and gene therapy, as well as combinations thereof. Immunotherapy, including, without limitation, adoptive cell therapy, the derivation of stem cells and/or dendritic cells, blood transfusions, lavages, and/or other treatments, including, without limitation, freezing a tumor.

In some embodiments, the one or more additional cancer therapies is chemotherapy, which can include administering one or more additional chemotherapeutic agents.

In certain embodiments, the additional chemotherapeutic agent is an immunomodulatory moiety, e.g., an immune checkpoint inhibitor. In certain of these embodiments, the immune checkpoint inhibitor targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155; e.g., CTLA-4 or PD1 or PD-L1). See, e.g., Postow, M. J. Clin. Oncol. 2015, 33, 1.

In certain of these embodiments, the immune checkpoint inhibitor is selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, and MGA271.

In certain embodiments, the additional chemotherapeutic agent is an alkylating agent. Alkylating agents are so named because of their ability to alkylate many nucleophilic functional groups under conditions present in cells, including, but not limited to cancer cells. In a further embodiment, an alkylating agent includes, but is not limited to, Cisplatin, carboplatin, mechlorethamine, cyclophosphamide, chlorambucil, ifosfamide and/or oxaliplatin. In an embodiment, alkylating agents can function by impairing cell function by forming covalent bonds with the amino, carboxyl, sulfhydryl, and phosphate groups in biologically important molecules or they can work by modifying a cell's DNA. In a further embodiment an alkylating agent is a synthetic, semisynthetic or derivative.

In certain embodiments, the additional chemotherapeutic agent is an anti-metabolite. Anti-metabolites masquerade as purines or pyrimidines, the building-blocks of DNA and in general, prevent these substances from becoming incorporated in to DNA during the “S” phase (of the cell cycle), stopping normal development and division. Anti-metabolites can also affect RNA synthesis. In an embodiment, an antimetabolite includes, but is not limited to azathioprine and/or mercaptopurine. In a further embodiment an anti-metabolite is a synthetic, semisynthetic or derivative.

In certain embodiments, the additional chemotherapeutic agent is a plant alkaloid and/or terpenoid. These alkaloids are derived from plants and block cell division by, in general, preventing microtubule function. In an embodiment, a plant alkaloid and/or terpenoid is a vinca alkaloid, a podophyllotoxin and/or a taxane. Vinca alkaloids, in general, bind to specific sites on tubulin, inhibiting the assembly of tubulin into microtubules, generally during the M phase of the cell cycle. In an embodiment, a vinca alkaloid is derived, without limitation, from the Madagascar periwinkle, Catharanthus roseus (formerly known as Vinca rosea). In an embodiment, a vinca alkaloid includes, without limitation, Vincristine, Vinblastine, Vinorelbine and/or Vindesine. In an embodiment, a taxane includes, but is not limited, to Taxol, Paclitaxel and/or Docetaxel. In a further embodiment a plant alkaloid or terpernoid is a synthetic, semisynthetic or derivative. In a further embodiment, a podophyllotoxin is, without limitation, an etoposide and/or teniposide. In an embodiment, a taxane is, without limitation, docetaxel and/or ortataxel. [021] In an embodiment, a cancer therapeutic is a topoisomerase. Topoisomerases are essential enzymes that maintain the topology of DNA. Inhibition of type I or type II topoisomerases interferes with both transcription and replication of DNA by upsetting proper DNA supercoiling. In a further embodiment, a topoisomerase is, without limitation, a type I topoisomerase inhibitor or a type II topoisomerase inhibitor. In an embodiment a type I topoisomerase inhibitor is, without limitation, a camptothecin. In another embodiment, a camptothecin is, without limitation, exatecan, irinotecan, lurtotecan, topotecan, BNP 1350, CKD 602, DB 67 (AR67) and/or ST 1481. In an embodiment, a type II topoisomerase inhibitor is, without limitation, epipodophyllotoxin. In a further embodiment an epipodophyllotoxin is, without limitation, an amsacrine, etoposid, etoposide phosphate and/or teniposide. In a further embodiment a topoisomerase is a synthetic, semisynthetic or derivative, including those found in nature such as, without limitation, epipodophyllotoxins, substances naturally occurring in the root of American Mayapple (Podophyllum peltatum).

In certain embodiments, the additional chemotherapeutic agent is a stilbenoid. In a further embodiment, a stilbenoid includes, but is not limited to, Resveratrol, Piceatannol, Pinosylvin, Pterostilbene, Alpha-Viniferin, Ampelopsin A, Ampelopsin E, Diptoindonesin C, Diptoindonesin F, Epsilon-Vinferin, Flexuosol A, Gnetin H, Hemsleyanol D, Hopeaphenol, Trans-Diptoindonesin B, Astringin, Piceid and Diptoindonesin A. In a further embodiment a stilbenoid is a synthetic, semisynthetic or derivative.

In certain embodiments, the additional chemotherapeutic agent is a cytotoxic antibiotic. In an embodiment, a cytotoxic antibiotic is, without limitation, an actinomycin, an anthracenedione, an anthracycline, thalidomide, dichloroacetic acid, nicotinic acid, 2-deoxyglucose and/or chlofazimine. In an embodiment, an actinomycin is, without limitation, actinomycin D, bacitracin, colistin (polymyxin E) and/or polymyxin B. In another embodiment, an antracenedione is, without limitation, mitoxantrone and/or pixantrone. In a further embodiment, an anthracycline is, without limitation, bleomycin, doxorubicin (Adriamycin), daunorubicin (daunomycin), epirubicin, idarubicin, mitomycin, plicamycin and/or valrubicin. In a further embodiment a cytotoxic antibiotic is a synthetic, semisynthetic or derivative.

In certain embodiments, the additional chemotherapeutic agent is selected from endostatin, angiogenin, angiostatin, chemokines, angioarrestin, angiostatin (plasminogen fragment), basement-membrane collagen-derived anti-angiogenic factors (tumstatin, canstatin, or arrestin), anti-angiogenic antithrombin III, signal transduction inhibitors, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-3), vasculostatin, vasostatin (calreticulin fragment) and the like.

In certain embodiments, the additional chemotherapeutic agent is selected from abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine.

In certain embodiments, the additional chemotherapeutic agent is platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In still other embodiments, the additional chemotherapeutic agent can be selected from those delineated in U.S. Pat. No. 7,927,613, which is incorporated herein by reference in its entirety.

In some embodiments, the additional therapeutic agent and/or regimen are those that can be used for treating other STING-associated conditions, e.g., type I interferonopathies (e.g., STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis and the like.

Non-limiting examples of additional therapeutic agents and/or regimens for treating rheumatoid arthritis include non-steroidal anti-inflammatory drugs (NSAIDs; e.g., ibuprofen and naproxen), corticosteroids (e.g, prednisone), disease-modifying antirheumatic drugs (DMARDs; e.g., methotrexate (Trexall®, Otrexup®, Rasuvo®, Rheumatrex®), leflunomide (Arava®), hydroxychloroquine (Plaquenil), PF-06650833, iguratimod, tofacitinib (Xeljanz®), ABBV-599, evobrutinib, and sulfasalazine (Azulfidine®)), and biologics (e.g., abatacept (Orencia®), adalimumab (Humira®), anakinra (Kineret®), certolizumab (Cimzia®), etanercept (Enbrel®), golimumab (Simponi®), infliximab (Remicade®), rituximab (Rituxan®), tocilizumab (Actemra®), vobarilizumab, sarilumab (Kevzara®), secukinumab, ABP 501, CHS-0214, ABC-3373, and tocilizumab (ACTEMRA®)).

Non-limiting examples of additional therapeutic agents and/or regimens for treating lupus include steroids, topical immunomodulators (e.g., tacrolimus ointment (Protopic®) and pimecrolimus cream (Elidel®)), thalidomide (Thalomid®), non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., evobrutinib, iberdomide, voclosporin, cenerimod, azathioprine (Imuran®), cyclophosphamide (Cytoxan®, Neosar®, Endoxan®), and cyclosporine (Neoral, Sandimmune®, Gengraf®), and mycophenolate mofetil) baricitinb, iguratimod, filogotinib, GS-9876, rapamycin, and PF-06650833), and biologics (e.g., belimumab (Benlysta®), anifrolumab, prezalumab, MEDIO700, obinutuzumab, vobarilizumab, lulizumab, atacicept, PF-06823859, and lupizor, rituximab, BT063, BI655064, BIIB059, aldesleukin (Proleukin®), dapirolizumab, edratide, IFN-α-kinoid, OMS721, RC18, RSLV-132, theralizumab, XmAb5871, and ustekinumab (Stelara®)). For example, non-limiting treatments for systemic lupus erythematosus include non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., iberdomide, voclosporin, azathioprine (Imuran®), cyclophosphamide (Cytoxan®, Neosar®, Endoxan®), and cyclosporine (Neoral, Sandimmune®, Gengraf®), and mycophenolate mofetil, baricitinb, filogotinib, and PF-06650833), and biologics (e.g., belimumab (Benlysta®), anifrolumab, prezalumab, MEDIO700, vobarilizumab, lulizumab, atacicept, PF-06823859, lupizor, rituximab, BT063, BI655064, BIIB059, aldesleukin (Proleukin®), dapirolizumab, edratide, IFN-α-kinoid, RC18, RSLV-132, theralizumab, XmAb5871, and ustekinumab (Stelara®)). As another example, non-limiting examples of treatments for cutaneous lupus include steroids, immunomodulators (e.g., tacrolimus ointment (Protopic®) and pimecrolimus cream (Elidel®)), GS-9876, filogotinib, and thalidomide (Thalomid®). Agents and regimens for treating drug-induced and/or neonatal lupus can also be administered.

Non-limiting examples of additional therapeutic agents and/or regimens for treating STING-associated vasculopathy with onset in infancy (SAVI) include JAK inhibitors (e.g., tofacitinib, ruxolitinib, filgotinib, and baricitinib).

Non-limiting examples of additional therapeutic agents and/or regimens for treating Aicardi-Goutières Syndrome (AGS) include physiotherapy, treatment for respiratory complications, anticonvulsant therapies for seizures, tube-feeding, nucleoside reverse transcriptase inhibitors (e.g., emtricitabine (e.g., Emtriva®), tenofovir (e.g., Viread®), emtricitabine/tenofovir (e.g., Truvada®), zidovudine, lamivudine, and abacavir), and JAK inhibitors (e.g., tofacitinib, ruxolitinib, filgotinib, and baricitinib).

Non-limiting examples of additional therapeutic agents and/or regimens for treating IBDs include 6-mercaptopurine, AbGn-168H, ABX464, ABT-494, adalimumab, AJM300, alicaforsen, AMG139, anrukinzumab, apremilast, ATR-107 (PF0530900), autologous CD34-selected peripheral blood stem cells transplant, azathioprine, bertilimumab, BI 655066, BMS-936557, certolizumab pegol (Cimzia®), cobitolimod, corticosteroids (e.g., prednisone, Methylprednisolone, prednisone), CP-690,550, CT-P13, cyclosporine, DIMS0150, E6007, E6011, etrasimod, etrolizumab, fecal microbial transplantation, figlotinib, fingolimod, firategrast (SB-683699) (formerly T-0047), GED0301, GLPG0634, GLPG0974, guselkumab, golimumab, GSK1399686, HMPL-004 (Andrographis paniculata extract), IMU-838, infliximab, Interleukin 2 (IL-2), Janus kinase (JAK) inhibitors, laquinimod, masitinib (AB1010), matrix metalloproteinase 9 (MMP 9) inhibitors (e.g., GS-5745), MEDI2070, mesalamine, methotrexate, mirikizumab (LY3074828), natalizumab, NNC 0142-0000-0002, NNC0114-0006, ozanimod, peficitinib (JNJ-54781532), PF-00547659, PF-04236921, PF-06687234, QAX576, RHB-104, rifaximin, risankizumab, RPC1063, SB012, SHP647, sulfasalazine, TD-1473, thalidomide, tildrakizumab (MK 3222), TJ301, TNF-Kinoid®, tofacitinib, tralokinumab, TRK-170, upadacitinib, ustekinumab, UTTR1147A, V565, vatelizumab, VB-201, vedolizumab, and vidofludimus.

Non-limiting examples of additional therapeutic agents and/or regimens for treating irritable bowel syndrome include alosetron, bile acid sequesterants (e.g., cholestyramine, colestipol, colesevelam), chloride channel activators (e.g., lubiprostone), coated peppermint oil capsules, desipramine, dicyclomine, ebastine, eluxadoline, farnesoid X receptor agonist (e.g., obeticholic acid), fecal microbiota transplantation, fluoxetine, gabapentin, guanylate cyclase-C agonists (e.g., linaclotide, plecanatide), ibodutant, imipramine, JCM-16021, loperamide, lubiprostone, nortriptyline, ondansetron, opioids, paroxetine, pinaverium, polyethylene glycol, pregabalin, probiotics, ramosetron, rifaximin, and tanpanor.

Non-limiting examples of additional therapeutic agents and/or regimens for treating scleroderma include non-steroidal anti-inflammatory drugs (NSAIDs; e.g., ibuprofen and naproxen), corticosteroids (e.g, prednisone), immunomodulators (e.g., azathioprine, methotrexate (Trexall®, Otrexup®, Rasuvo®, Rheumatrex®), cyclophosphamide (Cytoxan®, Neosar®, Endoxan®), and cyclosporine (Neoral®, Sandimmune®, Gengraf®), antithymocyte globulin, mycophenolate mofetil, intravenous immunoglobulin, rituximab, sirolimus, and alefacept), calcium channel blockers (e.g., nifedipine), alpha blockers, serotonin receptor antagonists, angiotensin II receptor inhibitors, statins, local nitrates, iloprost, phosphodiesterase 5 inhibitors (e.g., sildenafil), bosentan, tetracycline antibiotics, endothelin receptor antagonists, prostanoids, and tyrosine kinase inhibitors (e.g., imatinib, nilotinib and dasatinib).

Non-limiting examples of additional therapeutic agents and/or regimens for treating Crohn's Disease (CD) include adalimumab, autologous CD34-selected peripheral blood stem cells transplant, 6-mercaptopurine, azathioprine, certolizumab pegol (Cimzia®), corticosteroids (e.g., prednisone), etrolizumab, E6011, fecal microbial transplantation, figlotinib, guselkumab, infliximab, IL-2, JAK inhibitors, matrix metalloproteinase 9 (MMP 9) inhibitors (e.g., GS-5745), MEDI2070, mesalamine, methotrexate, natalizumab, ozanimod, RHB-104, rifaximin, risankizumab, SHP647, sulfasalazine, thalidomide, upadacitinib, V565, and vedolizumab.

Non-limiting examples of additional therapeutic agents and/or regimens for treating UC include AbGn-168H, ABT-494, ABX464, apremilast, PF-00547659, PF-06687234, 6-mercaptopurine, adalimumab, azathioprine, bertilimumab, brazikumab (MEDI2070), cobitolimod, certolizumab pegol (Cimzia®), CP-690,550, corticosteroids (e.g., multimax budesonide, Methylprednisolone), cyclosporine, E6007, etrasimod, etrolizumab, fecal microbial transplantation, figlotinib, guselkumab, golimumab, IL-2, IMU-838, infliximab, matrix metalloproteinase 9 (MMP9) inhibitors (e.g., GS-5745), mesalamine, mesalamine, mirikizumab (LY3074828), RPC1063, risankizumab (BI 6555066), SHP647, sulfasalazine, TD-1473, TJ301, tildrakizumab (MK 3222), tofacitinib, tofacitinib, ustekinumab, UTTR1147A, and vedolizumab.

Non-limiting examples of additional therapeutic agents and/or regimens for treating autoimmune colitis include corticosteroids (e.g., budesonide, prednisone, prednisolone, Beclometasone dipropionate), diphenoxylate/atropine, infliximab, loperamide, mesalamine, TIP60 inhibitors (see, e.g., U.S. Patent Application Publication No. 2012/0202848), and vedolizumab.

Non-limiting examples of additional therapeutic agents and/or regimens for treating iatrogenic autoimmune colitis include corticosteroids (e.g., budesonide, prednisone, prednisolone, Beclometasone dipropionate), diphenoxylate/atropine, infliximab, loperamide, TIP60 inhibitors (see, e.g., U.S. Patent Application Publication No. 2012/0202848), and vedolizumab.

Non-limiting examples of additional therapeutic agents and/or regimens for treating colitis induced by one or more chemotherapeutics agents include corticosteroids (e.g., budesonide, prednisone, prednisolone, beclometasone dipropionate), diphenoxylate/atropine, infliximab, loperamide, mesalamine, TIP60 inhibitors (see, e.g., U.S. Patent Application Publication No. 2012/0202848), and vedolizumab.

Non-limiting examples of additional therapeutic agents and/or regimens for treating colitis induced by treatment with adoptive cell therapy include corticosteroids (e.g., budesonide, prednisone, prednisolone, beclometasone dipropionate), diphenoxylate/atropine, infliximab, loperamide, TIP60 inhibitors (see, e.g., U.S. Patent Application Publication No. 2012/0202848), and vedolizumab.

Non-limiting examples of additional therapeutic agents and/or regimens for treating colitis associated with one or more alloimmune diseases include corticosteroids (e.g., budesonide, prednisone, prednisolone, beclometasone dipropionate), sulfasalazine, and eicopentaenoic acid.

Non-limiting examples of additional therapeutic agents and/or regimens for treating radaiation enteritis include teduglutide, amifostine, angiotensin-converting enzyme (ACE) inhibitors (e.g., benazepril, captopril, enalapril, fosinopril, lisinopril, moexipril, perindopril, quinapril, ramipril, and trandolapril), probiotics, selenium supplementation, statins (e.g., atorvastatin, fluvastatin, lovastatin, pravastatin, rosuvastatin, simvastatin, and pitavastatin), sucralfate, and vitamin E.

Non-limiting examples of additional therapeutic agents and/or regimens for treating collagenous colitis include 6-mercaptopurine, azathaioprine, bismuth subsalicate, Boswellia serrata extract, cholestyramine, colestipol, corticosteroids (e.g., budesonide, prednisone, prednisolone, beclometasone dipropionate), loperamide, mesalamine, methotrexate, probiotics, and sulfasalazine.

Non-limiting examples of additional therapeutic agents and/or regimens for treating lyphocytic colitis include 6-mercaptopurine, azathioprine, bismuth subsalicylate, cholestyramine, colestipol, corticosteroids (e.g., budesonide, prednisone, prednisolone, beclometasone dipropionate), loperamide, mesalamine, methotrexate, and sulfasalazine.

Non-limiting examples of additional therapeutic agents and/or regimens for treating microscopic colitis include 6-mercaptopurine, azathioprine, bismuth subsalicylate, Boswellia serrata extract, cholestyramine, colestipol, corticosteroids (e.g., budesonide, prednisone, prednisolone, beclometasone dipropionate), fecal microbial transplantation, loperamide, mesalamine, methotrexate, probiotics, and sulfasalazine.

Non-limiting examples of additional therapeutic agents and/or regimens for treating alloimmune disease include intrauterine platelet transfusions, intravenous immunoglobin, maternal steroids, abatacept, alemtuzumab, alpha1-antitrypsin, AMG592, antithymocyte globulin, barcitinib, basiliximab, bortezomib, brentuximab, cannabidiol, corticosteroids (e.g., methylprednisone, prednisone), cyclosporine, dacilzumab, defribrotide, denileukin diftitox, glasdegib, ibrutinib, IL-2, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

Non-limiting examples of additional therapeutic agents and/or regimens for treating multiple sclerosis (MS) include alemtuzumab (Lemtrada®), ALKS 8700, amiloride, ATX-MS-1467, azathioprine, baclofen (Lioresal®), beta interferons (e.g., IFN-β-1a, IFN-β-1b), cladribine, corticosteroids (e.g., methylprednisolone), daclizumab, dimethyl fumarate (Tecfidera®), fingolimod (Gilenya®), fluoxetine, glatiramer acetate (Copaxone®), hydroxychloroquine, ibudilast, idebenone, laquinimod, lipoic acid, losartan, masitinib, MDD1003 (biotin), mitoxantrone, montelukast, natalizumab (Tysabri®), NeuroVax™, ocrelizumab, ofatumumab, pioglitazone, and RPC1063.

Non-limiting examples of additional therapeutic agents and/or regimens for treating graft-vs-host disease include abatacept, alemtuzumab, alpha1-antitrypsin, AMG592, antithymocyte globulin, barcitinib, basiliximab, bortezomib, brentuximab, cannabidiol, corticosteroids (e.g., methylprednisone, prednisone), cyclosporine, dacilzumab, defribrotide, denileukin diftitox, glasdegib, ibrutinib, IL-2, imatinib, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

Non-limiting examples of additional therapeutic agents and/or regimens for treating acute graft-vs-host disease include alemtuzumab, alpha-1 antitrypsin, antithymocyte globulin, basiliximab, brentuximab, corticosteroids (e.g., methylprednisone, prednisone), cyclosporine, dacilzumab, defribrotide, denileukin diftitox, ibrutinib, infliximab, itacitinib, LBH589, mycophenolate mofetil, natalizumab, neihulizumab, pentostatin, photopheresis, ruxolitinib, sirolimus, tacrolimus, and tocilizumab.

Non-limiting examples of additional therapeutic agents and/or regimens for treating chronic graft vs. host disease include abatacept, alemtuzumab, AMG592, antithymocyte globulin, basiliximab, bortezomib, corticosteroids (e.g., methylprednisone, prednisone), cyclosporine, dacilzumab, denileukin diftitox, glasdegib, ibrutinib, IL-2, imatinib, infliximab, mycophenolate mofetil, pentostatin, photobiomodulation, photopheresis, ruxolitinib, sirolimus, sonidegib, tacrolimus, tocilizumab, and vismodegib.

Non-limiting examples of additional therapeutic agents and/or regimens for treating celiac disease include AMG 714, AMY01, Aspergillus niger prolyl endoprotease, BL-7010, CALY-002, GBR 830, Hu-Mik-Beta-1, IMGX003, KumaMax, Larazotide Acetate, Nexvan2®, pancrelipase, TIMP-GLIA, vedolizumab, and ZED1227.

Non-limiting examples of additional therapeutic agents and/or regimens for treating psoriasis include topical corticosteroids, topical crisaborole/AN2728, topical SNA-120, topical SAN021, topical tapinarof, topical tocafinib, topical IDP-118, topical M518101, topical calcipotriene and betamethasone dipropionate (e.g., MC2-01 cream and Taclonex®), topical P-3073, topical LEO 90100 (Enstilar®), topical betamethasone dipropriate (Sernivo®), halobetasol propionate (Ultravate®), vitamin D analogues (e.g., calcipotriene (Dovonex®) and calcitriol (Vectical®)), anthralin (e.g., Dritho-Scalp® and Dritho-crème®), topical retinoids (e.g., tazarotene (e.g., Tazorac® and Avage®)), calcineurin inhibitors (e.g., tacrolimus (Prograf®) and pimecrolimus (Elidel®)), salicylic acid, coal tar, moisturizers, phototherapy (e.g., exposure to sunlight, UVB phototherapy, narrow band UVB phototherapy, Goeckerman therapy, psoralen plus ultraviolet A (PUVA) therapy, and excimer laser), retinoids (e.g., acitretin (Soriatane®)), methotrexate (Trexall®, Otrexup®, Rasuvo®, Rheumatrex®), Apo805K1, baricitinib, FP187, KD025, prurisol, VTP-43742, XP23829, ZPL-389, CF101 (piclidenoson), LAS41008, VPD-737 (serlopitant), upadacitinib (ABT-494), aprmilast, tofacitibin, cyclosporine (Neoral®, Sandimmune®, Gengraf®), biologics (e.g., etanercept (Enbrel®), entanercept-szzs (Elrezi®), infliximab (Remicade®), adalimumab (Humira®), adalimumab-adbm (Cyltezo®), ustekinumab (Stelara®), golimumab (Simponi®), apremilast (Otezla®), secukinumab (Cosentyx®), certolixumab pegol, secukinumab, tildrakizumab-asmn, infliximab-dyyb, abatacept, ixekizumab (Taltz®), ABP 710, BCD-057, BI695501, bimekizumab (UCB4940), CHS-1420, GP2017, guselkumab (CNTO 1959), HD203, M923, MSB11022, Mirikizumab (LY3074828), PF-06410293, PF-06438179, risankizumab (BI655066), SB2, SB4, SB5, siliq (brodalumab), namilumab (MT203, tildrakizumab (MK-3222), and ixekizumab (Taltz®)), thioguanine, and hydroxyurea (e.g., Droxia® and Hydrea®).

Non-limiting examples of additional therapeutic agents and/or regimens for treating cutaneous T-cell lymphoma include phototherapy (e.g., exposure to sunlight, UVB phototherapy, narrow band UVB phototherapy, Goeckerman therapy, psoralen plus ultraviolet A (PUVA) therapy, and excimer laser), extracorporeal photopheresis, radiation therapy (e.g., spot radiation and total skin body electron beam therapy), stem cell transplant, corticosteroids, imiquimod, bexarotene gel, topical bis-chloroethyl-nitrourea, mechlorethamine gel, vorinostat (Zolinza®), romidepsin (Istodax®), pralatrexate (Folotyn®) biologics (e.g., alemtuzumab (Campath®), brentuximab vedotin (SGN-35), mogamulizumab, and IPH4102).

Non-limiting examples of additional therapeutic agents and/or regimens for treating uveitis include corticosteroids (e.g., intravitreal triamcinolone acetonide injectable suspensions), antibiotics, antivirals (e.g., acyclovir), dexamethasone, immunomodulators (e.g., tacrolimus, leflunomide, cyclophosphamide (Cytoxan®, Neosar®, Endoxan®), and cyclosporine (Neoral®, Sandimmune®, Gengraf®), chlorambucil, azathioprine, methotrexate, and mycophenolate mofetil), biologics (e.g., infliximab (Remicade®), adalimumab (Humira®), etanercept (Enbrel®), golimumab (Simponi®), certolizumab (Cimzia®), rituximab (Rituxan®), abatacept (Orencia®), basiliximab (Simulect®), anakinra (Kineret®), canakinumab (Ilaris®), gevokixumab (XOMA052), tocilizumab (Actemra®), alemtuzumab (Campath®), efalizumab (Raptiva®), LFG316, sirolimus (Santen®), abatacept, sarilumab (Kevzara®), and daclizumab (Zenapax®)), cytotoxic drugs, surgical implant (e.g., fluocinolone insert), and vitrectomy.

Non-limiting examples of additional therapeutic agents and/or regimens for treating mucositis include AG013, SGX942 (dusquetide), amifostine (Ethyol®), cryotherapy, cepacol lonzenges, capsaicin lozenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, Chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, granules comprising Vaccinium myrtillus extract, Macleaya cordata alkaloids and Echinacea angustifolia extract (e.g., SAMITAL®), and gastrointestinal cocktail (an acid reducer such aluminum hydroxide and magnesium hydroxide (e.g., Maalox), an antifungal (e.g., nystatin), and an analgesic (e.g., hurricane liquid)). For example, non-limiting examples of treatments for oral mucositis include AG013, amifostine (Ethyol®), cryotherapy, cepacol lonzenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, Chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, and gastrointestinal cocktail (an acid reducer such aluminum hydroxide and magnesium hydroxide (e.g., Maalox), an antifungal (e.g., nystatin), and an analgesic (e.g., hurricane liquid)). As another example, non-limiting examples of treatments for esophageal mucositis include xylocaine (e.g., gel viscous Xylocaine 2%). As another example, treatments for intestinal mucositis, treatments to modify intestinal mucositis, and treatments for intestinal mucositis signs and symptoms include gastrointestinal cocktail (an acid reducer such aluminum hydroxide and magnesium hydroxide (e.g., Maalox), an antifungal (e.g., nystatin), and an analgesic (e.g., hurricane liquid)).

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

Patient Selection

In some embodiments, the methods described herein further include the step of identifying a subject (e.g., a patient) in need of such treatment (e.g., by way of biopsy, endoscopy, or other conventional method known in the art). In certain embodiments, the STING protein can serve as a biomarker for certain types of cancer, e.g., colon cancer and prostate cancer. In other embodiments, identifying a subject can include assaying the patient's tumor microenvironment for the absence of T-cells and/or presence of exhausted T-cells, e.g., patients having one or more cold tumors. Such patients can include those that are resistant to treatment with checkpoint inhibitors. In certain embodiments, such patients can be treated with a chemical entity herein, e.g., to recruit T-cells into the tumor, and in some cases, further treated with one or more checkpoint inhibitors, e.g., once the T-cells become exhausted.

In some embodiments, the chemical entities, methods, and compositions described herein can be administered to certain treatment-resistant patient populations (e.g., patients resistant to checkpoint inhibitors; e.g., patients having one or more cold tumors, e.g., tumors lacking T-cells or exhausted T-cells).

Compound Preparation

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and RGM. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. The starting materials used in preparing the compounds of the invention are known, made by known methods, or are commercially available. The skilled artisan will also recognize that conditions and reagents described herein that can be interchanged with alternative art-recognized equivalents. For example, in many reactions, triethylamine can be interchanged with other bases, such as non-nucleophilic bases (e.g. diisopropylamine, 1,8-diazabicycloundec-7-ene, 2,6-di-tert-butylpyridine, or tetrabutylphosphazene).

The skilled artisan will recognize a variety of analytical methods that can be used to characterize the compounds described herein, including, for example, ¹H NMR, heteronuclear NMR, mass spectrometry, liquid chromatography, and infrared spectroscopy. The foregoing list is a subset of characterization methods available to a skilled artisan and is not intended to be limiting.

To further illustrate the foregoing, the following non-limiting, exemplary synthetic schemes are included. Variations of these examples within the scope of the claims are within the purview of one skilled in the art and are considered to fall within the scope of the invention as described, and claimed herein. The reader will recognize that the skilled artisan, provided with the present disclosure, and skill in the art is able to prepare and use the invention without exhaustive examples.

The following abbreviations have the indicated meanings:

### EXAMPLES

For illustrative purposes, exemplary general methods for synthesizing compounds of Formula I are depicted in Schemes 1 and 2.

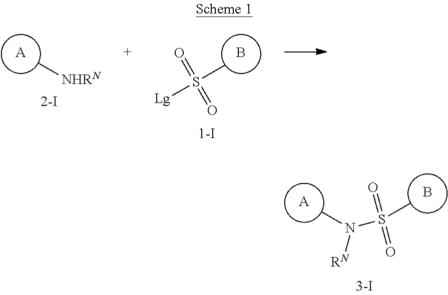

Referring to Scheme 1, a compound of Formula I (shown as compound 3-I in Scheme 1) wherein L^{AB} is —N(R^{N})S(O)₂—* as defined for Formula I; and R^{N}, A, and B are as defined for Formula I can be prepared through the coupling of 1-I and amine 2-I (in 1-I, B is as defined for Formula I, and Lg is a leaving atom (e.g., Cl, Br) or leaving group (e.g., OMs, OTf, OTs); and in 2-I, R^{N} and A are as defined for Formula I).

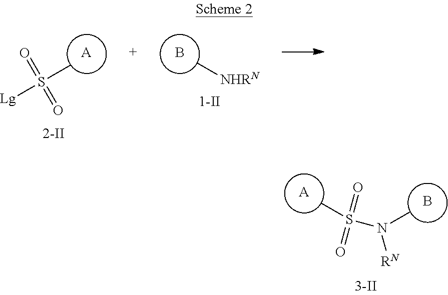

Referring to Scheme 2, a compound of Formula I (shown as compound 3-II in Scheme 2) wherein L^{AB} is —S(O)₂N(R^{N})—* as defined for Formula I; and R^{N}, A, and B are as defined for Formula I can be prepared through the coupling of 1-II and 2-II (in 1-IT, R^{N} and B are as defined for Formula I; and in 2-IT, A is as defined for Formula I, and Lg is a leaving atom (e.g., Cl, Br) or leaving group (e.g., OMs, OTf, OTs)).

The following compounds are prepared according to methods shown in Schemes 1 and 2:

| Compound | Structure |
|---|---|
| 101 | 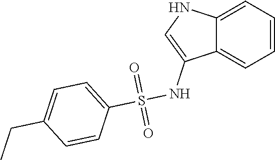 [SMILES: CCc1ccc(S(=O)(=O)Nc2c[nH]c3ccccc23)cc1] |
| 102 | 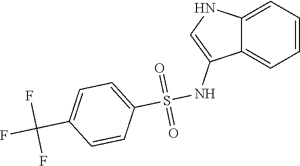 [SMILES: O=S(=O)(Nc1c[nH]c2ccccc12)c1ccc(C(F)(F)F)cc1] |
| 103 | 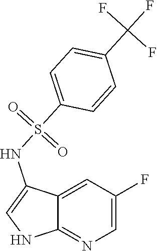 [SMILES: O=S(=O)(Nc1c[nH]c2ncc(F)cc12)c1ccc(C(F)(F)F)cc1] |
| 104 | 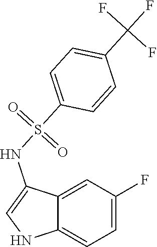 [SMILES: O=S(=O)(Nc1c[nH]c2ccc(F)cc12)c1ccc(C(F)(F)F)cc1] |
| 105 | 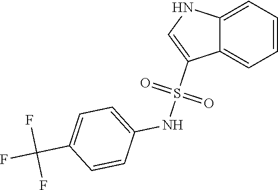 [SMILES: O=S(=O)(Nc1ccc(C(F)(F)F)cc1)c1c[nH]c2ccccc12] |
| 106 | 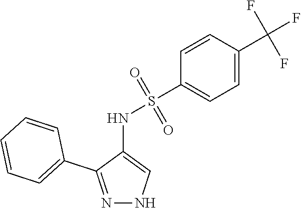 |
| 107 | 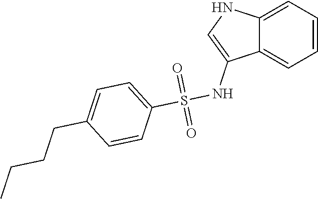 [SMILES: CCCCc1ccc(S(=O)(=O)Nc2c[nH]c3ccccc23)cc1] |
| 108 | 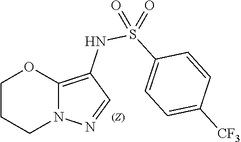 [SMILES: O=S(=O)(Nc1cnn2c1OCCC2)c1ccc(C(F)(F)F)cc1] |
| 109 | 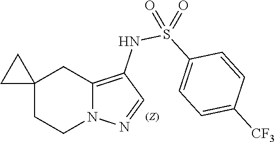 [SMILES: O=S(=O)(Nc1cnn2c1CC1(CC2)CC1)c1ccc(C(F)(F)F)cc1] |
| 110 | 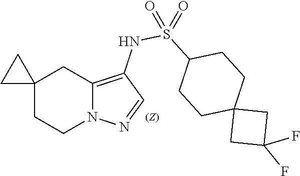 [SMILES: O=S(=O)(Nc1cnn2c1CC1(CC2)CC1)C1CCC2(CC1)CC(F)(F)C2] |
| 111 | 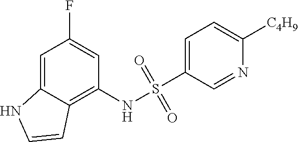 |
| 112 | 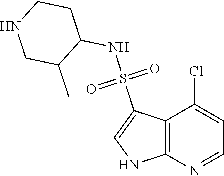 [SMILES: CC1CNCCC1NS(=O)(=O)c1c[nH]c2nccc(Cl)c12] |

### Abbreviation of Chemical Terms

DCM=dichloromethane

### DMF=N,N-dimethylformamide

HATU=N-[(Dimethyl amino)-1H-1,2,3-triazolo-[4,5-b]pyridin-1l-ylmethylene]-N-methylmethanaminium hexafluorophosphate N-oxide
HPLC=high-performance liquid chromatography
LCMS=liquid chromatography-mass spectrometry
NMR=nuclear magnetic resonance
DIEA=N-ethyl-N-isopropylpropan-2-amine
FA=formic acid
TFA=trifluoroacetic acid

### Speedvac=Savant SC250EXP SpeedVac Concentrator

### LCMS Analysis Condition

### Method A

Instrument: Agilent LCMS system equipped with DAD and ELSD detector
Ion mode: Positive
Column: Waters X-Bridge C18, 50*2.1 mm*5 m or equivalent
Mobile Phase: A: H₂O (0.04% TFA); B: CH₃CN (0.02% TFA)
Gradient: 4.5 min gradient method, actual method would depend on c log P of compound.
Flow Rate: 0.6 mL/min or 0.8 mL/min

### Column Temp: 40° C. or 50° C.

### UV: 220 nm

### Method B

Instrument: Agilent LCMS system equipped with DAD and ELSD detector
Ion mode: Positive
Column: Waters X-Bridge Shield RP18, 50*2.1 mm*5 m or equivalent
Mobile Phase: A: H₂O (0.05% NH₃.H₂O) or 10 mM ammonia bicarbonate; B: CH₃CN
Gradient: 4.5 min gradient method; actual method would depend on the c log P of the compound.
Flow Rate: 0.6 mL/min or 0.8 mL/min

### Column Temp: 40° C.

### UV: 220 nm

Prep. HPLC Condition

### Instrument:

### 1. GILSON 281 and Shimadzu LCMS 2010A

### 2. GILSON 215 and Shimadzu LC-20AP

### 3. GILSON 215

### Mobile Phase:

A: NH₄OH/H₂O=0.05% v/v; B: ACN
A: FA/H₂O=0.225% v/v; B: ACN

### Column

Xtimate C18 150*25 mm*5 μm
Flow rate: 25 mL/min or 30 mL/min
Monitor wavelength: 220&254 nm
Gradient: actual method would depend on clog P of compound

### Detector: MS Trigger or UV

### Example 1: Synthesis of Compound 113

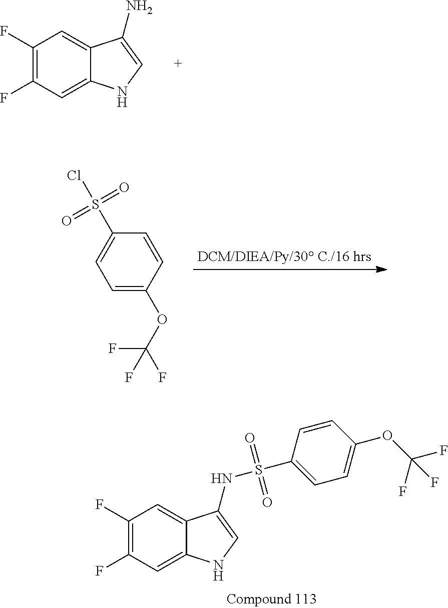 [SMILES: Nc1c[nH]c2cc(F)c(F)cc12 ; O=S(=O)(Cl)c1ccc(OC(F)(F)F)cc1 ; O=S(=O)(Nc1c[nH]c2cc(F)c(F)cc12)c1ccc(OC(F)(F)F)cc1]

### Procedure 2

### Synthesis of N-(5,6-difluoro-1H-indol-3-yl)-4-(trifluoromethoxy) benzenesulfonamide

5,6-difluoro-1H-indol-3-amine (42.8 mg, 0.255 mmol, 1.0 equiv.) was dissolved in DCM (2.0 mL). DIEA (168 μl, 1.02 mmol, 4.0 equiv.) and pyridine (82 μl, 1.02 mmol, 4.0 equiv.) were then added. A solution of 4-(trifluoromethoxy) benzene-1-sulfonyl chloride (72.8 mg, 280.0 μmol, 1.1 equiv.) dissolved in 1.0 mL DCM was added to the reaction mixture. The reaction mixture was stirred at 30° C. for 16 hours. The reaction mixture was concentrated by Speedvac. The resulting residue was purified by prep HPLC to provide N-(5,6-difluoro-1H-indol-3-yl)-4-(trifluoromethoxy) benzenesulfonamide (10.2 mg, 26.0 μmol). MS-ESI, 393.1 [M+H⁺].

¹H NMR (400 MHz, DMSO-d₆) δ ppm 11.19 (br s, 1H) 9.81 (br s, 1H) 7.76 (d, 2H) 7.48 (br d, 2H) 7.29 (dd, 1H) 7.18 (d, 1H) 6.95 (dd, 1H)

TABLE E1
The compounds in Table E1 were prepared using the above procedure.

| | | | | LC-MS, |
|---|---|---|---|---|
| Ex- | Com- | | | MS-ESI, |
| ample | pound | | IUPAC | — |
| # | # | Final compound | Name | [M + H⁺]. |
| 2 | 114 | 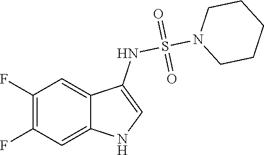 [SMILES: O=S(=O)(Nc1c[nH]c2cc(F)c(F)cc12)N1CCCCC1] | N-(5,6- difluoro- 1H-indol- 3-yl) piperidine-1- sulfonamide | |
| 3 | 115 | 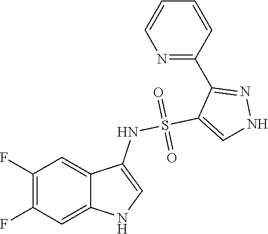 [SMILES: O=S(=O)(Nc1c[nH]c2cc(F)c(F)cc12)c1c[nH]nc1-c1ccccn1] | N-(5,6- difluoro- 1H-indol- 3-yl)-3- (pyridin-2- yl)-1H- pyrazole- 4- sulfonamide | |
| 4 | 116 | 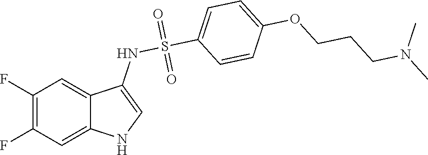 [SMILES: CN(C)CCCOc1ccc(S(=O)(=O)Nc2c[nH]c3cc(F)c(F)cc23)cc1] | N-(5,6- difluoro- 1H-indol- 3-yl)-4-(3- (dimethylamino) propoxy) benzenesulfonamide | |
| 5 | 117 | 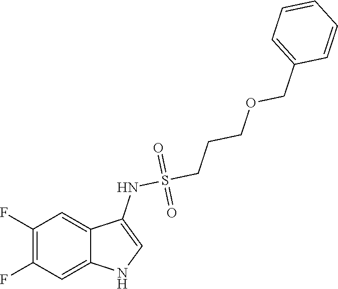 [SMILES: O=S(=O)(CCCOCc1ccccc1)Nc1c[nH]c2cc(F)c(F)cc12] | 3- (benzyloxy)- N-(5,6- difluoro- 1H-indol-3- yl)propane-1- sulfonamide | |
| 6 | 118 | 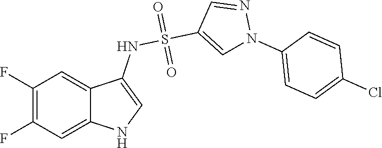 [SMILES: O=S(=O)(Nc1c[nH]c2cc(F)c(F)cc12)c1cnn(-c2ccc(Cl)cc2)c1] | 1-(4- chlorophenyl)- N-(5,6- difluoro- 1H-indol- 3-yl)-1H- pyrazole-4- sulfonamide | |
| 7 | 119 | 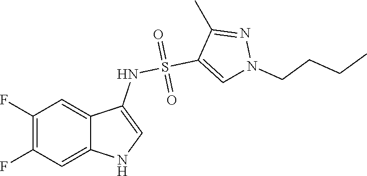 [SMILES: CCCCn1cc(S(=O)(=O)Nc2c[nH]c3cc(F)c(F)cc23)c(C)n1] | 1-butyl-N- (5,6- difluoro- 1H-indol- 3-yl)-3- methyl- 1H- pyrazole- 4- sulfonamide | |
| 8 | 120 | 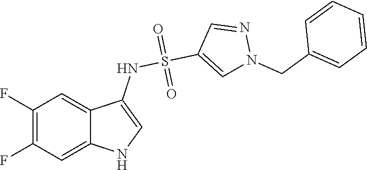 [SMILES: O=S(=O)(Nc1c[nH]c2cc(F)c(F)cc12)c1cnn(Cc2ccccc2)c1] | 1-benzyl- N-(5,6- difluoro- 1H-indol- 3-yl)-1H- pyrazole- 4- sulfonamide | 389.2 |
| 9 | 121 | 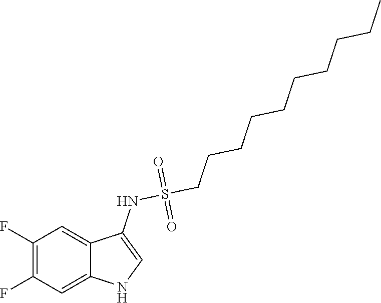 [SMILES: CCCCCCCCCCS(=O)(=O)Nc1c[nH]c2cc(F)c(F)cc12] | N-(5,6- difluoro- 1H-indol- 3- yl)decane- 1- sulfonamide | |
| 10 | 122 | 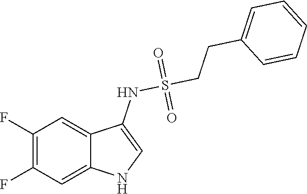 [SMILES: O=S(=O)(CCc1ccccc1)Nc1c[nH]c2cc(F)c(F)cc12] | N-(5,6- difluoro- 1H-indol- 3-yl)-2- phenylethane- sulfonamide | |
| 11 | 123 | 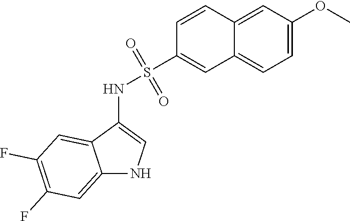 | N-(5,6- difluoro- 1H-indol- 3-yl)-6- methoxy- naphthalene-2- sulfonamide | |
| 12 | 124 | 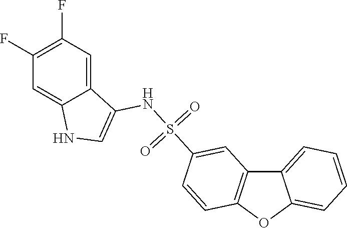 [SMILES: O=S(=O)(Nc1c[nH]c2cc(F)c(F)cc12)c1ccc2oc3ccccc3c2c1] | N-(5,6- difluoro- 1H-indol- 3- yl)dibenzo [b,d]furan-2- sulfonamide | |
| 13 | 125 | 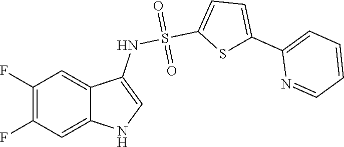 [SMILES: O=S(=O)(Nc1c[nH]c2cc(F)c(F)cc12)c1ccc(-c2ccccn2)s1] | N-(5,6- difluoro- 1H-indol- 3-yl)-5- (pyridin-2- yl)thiophene-2- sulfonamide | |
| 14 | 126 | 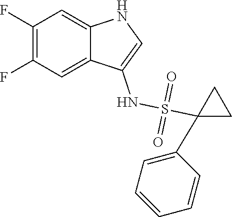 [SMILES: O=S(=O)(Nc1c[nH]c2cc(F)c(F)cc12)C1(c2ccccc2)CC1] | N-(5,6- difluoro- 1H-indol- 3-yl)-1- phenylcyclopropane- 1-sulfonamide | |
| 15 | 127 | 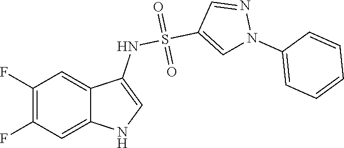 [SMILES: O=S(=O)(Nc1c[nH]c2cc(F)c(F)cc12)c1cnn(-c2ccccc2)c1] | N-(5,6- difluoro- 1H-indol- 3-yl)-1- phenyl-1H- pyrazole-4- sulfonamide | 375.1 |
| 16 | 128 | 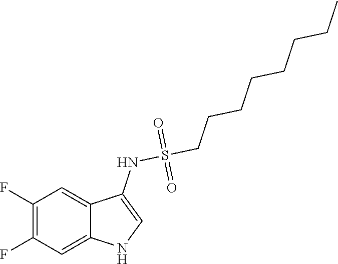 [SMILES: CCCCCCCCS(=O)(=O)Nc1c[nH]c2cc(F)c(F)cc12] | N-(5,6- difluoro- 1H-indol-3- yl)octane-1- sulfonamide | 345 |
| 17 | 129 | 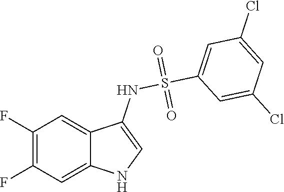 [SMILES: O=S(=O)(Nc1c[nH]c2cc(F)c(F)cc12)c1cc(Cl)cc(Cl)c1] | 3,5- dichloro- N-(5,6- difluoro- 1H-indol-3-yl) benzenesulfonamide | 378.8 |
| 18 | 130 | 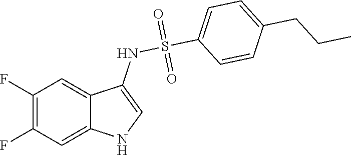 [SMILES: CCCc1ccc(S(=O)(=O)Nc2c[nH]c3cc(F)c(F)cc23)cc1] | N-(5,6- difluoro- 1H-indol- 3-yl)-4- propylbenzene- sulfonamide | 351.1 |
| 19 | 131 | 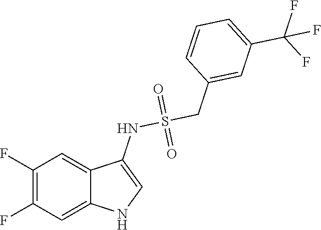 [SMILES: O=S(=O)(Cc1cccc(C(F)(F)F)c1)Nc1c[nH]c2cc(F)c(F)cc12] | N-(5,6- difluoro- 1H-indol- 3-yl)-1-(3- (trifluoromethyl) phenyl) methanesulfonamide | 391.1 |
| 20 | 132 | 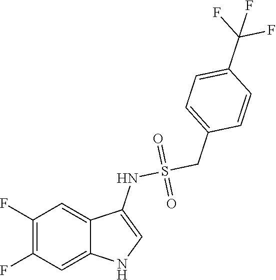 [SMILES: O=S(=O)(Cc1ccc(C(F)(F)F)cc1)Nc1c[nH]c2cc(F)c(F)cc12] | N-(5,6- difluoro- 1H-indol- 3-yl)-1-(4- (trifluoromethyl) phenyl) methanesulfonamide | 391 |
| 21 | 133 | 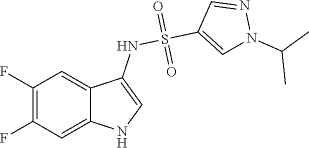 [SMILES: CC(C)n1cc(S(=O)(=O)Nc2c[nH]c3cc(F)c(F)cc23)cn1] | N-(5,6- difluoro- 1H-indol- 3-yl)-1- isopropyl-1H- pyrazole-4- sulfonamide | 341.1 |
| 22 | 134 | 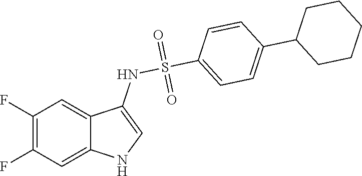 [SMILES: O=S(=O)(Nc1c[nH]c2cc(F)c(F)cc12)c1ccc(C2CCCCC2)cc1] | 4- cyclohexyl-N- (5,6- difluoro- 1H-indol-3-yl) benzenesulfonamide | 391.2 |
| 23 | 135 | 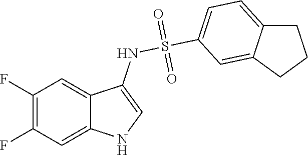 [SMILES: O=S(=O)(Nc1c[nH]c2cc(F)c(F)cc12)c1ccc2c(c1)CCC2] | N-(5,6- difluoro- 1H-indol- 3-yl)-2,3- dihydro-1H- indene-5- sulfonamide | 349 |
| 24 | 136 | 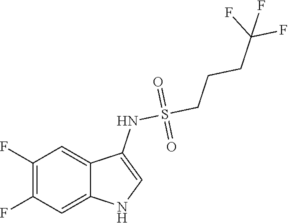 [SMILES: O=S(=O)(CCCC(F)(F)F)Nc1c[nH]c2cc(F)c(F)cc12] | N-(5,6- difluoro- 1H-indol- 3-yl)- 4,4,4- trifluorobutane-1- sulfonamide | 343.1 |
| 25 | 137 | 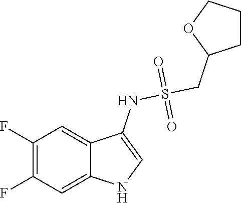 [SMILES: O=S(=O)(CC1CCCO1)Nc1c[nH]c2cc(F)c(F)cc12] | N-(5,6- difluoro- 1H-indol- 3-yl)-1- (tetrahydrofuran- 2-yl) methanesulfonamide | 317.1 |
| 26 | 138 | 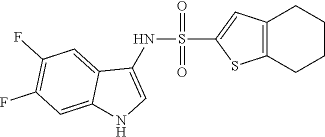 [SMILES: O=S(=O)(Nc1c[nH]c2cc(F)c(F)cc12)c1cc2c(s1)CCCC2] | N-(5,6- difluoro- 1H-indol- 3-yl)- 4,5,6,7- tetrahydrobenzo [b]thiophene-2- sulfonamide | 369.1 |
| 27 | 139 | 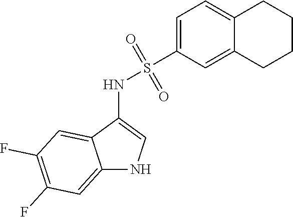 | N-(5,6- difluoro- 1H-indol- 3-yl)- 5,6,7,8- tetrahydro- naphthalene-2- sulfonamide | 363.1 |
| 28 | 140 | 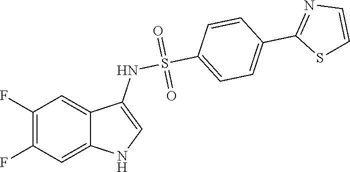 [SMILES: O=S(=O)(Nc1c[nH]c2cc(F)c(F)cc12)c1ccc(-c2nccs2)cc1] | N-(5,6- difluoro- 1H-indol- 3-yl)-4- (thiazol-2-yl) benzenesulfonamide | 392.1 |
| 29 | 141 | 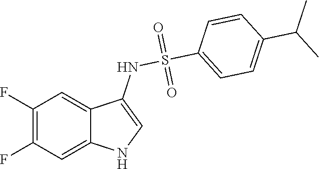 [SMILES: CC(C)c1ccc(S(=O)(=O)Nc2c[nH]c3cc(F)c(F)cc23)cc1] | N-(5,6- difluoro- 1H-indol- 3-yl)-4- isopropylbenzene- sulfonamide | 351.2 |
| 30 | 142 | 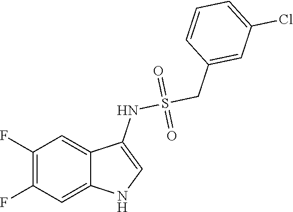 [SMILES: O=S(=O)(Cc1cccc(Cl)c1)Nc1c[nH]c2cc(F)c(F)cc12] | 1-(3- chlorophenyl)- N-(5,6- difluoro- 1H-indol-3-yl) methanesulfonamide | |

### Example 31: The Synthesis of Compound 143

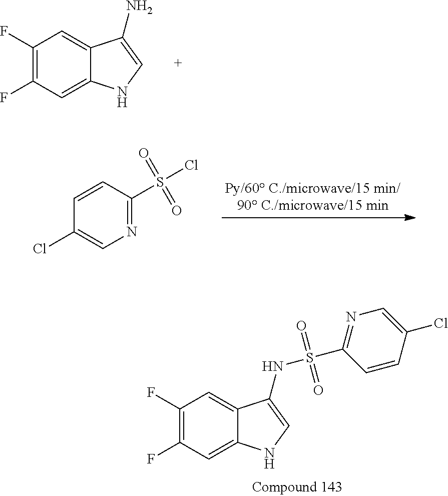 [SMILES: Nc1c[nH]c2cc(F)c(F)cc12 ; O=S(=O)(Cl)c1ccc(Cl)cn1 ; O=S(=O)(Nc1c[nH]c2cc(F)c(F)cc12)c1ccc(Cl)cn1]

### Synthesis of 5-chloro-N-(5,6-difluoro-1H-indol-3-yl)pyridine-3-sulfonamide

5,6-difluoro-1H-indol-3-amine (8.4 mg, 50.0 μmol, 1.0 equiv.) and 5-chloropyridine-2-sulfonyl chloride (11.5 mg, 55.0 μmol, 1.1 equiv.) were taken up into a microwave tube were dissolved in pyridine (0.3 mL). The sealed tube was heated at 60° C. for 15 mins under microwave condition. After the reaction mixture cooled down to 55° C., then the mixture was heated again at 90° C. for 15 mins under microwave condition. 6 parallel reaction batches were carried out. The reaction mixture of 6 batches was combined together and concentrated by Speedvac. The residue was purified by prep HPLC to give 5-chloro-N-(5,6-difluoro-1H-indol-3-yl)pyridine-2-sulfonamide (27.2 mg, 79.3 μmol). MS-ESI, 344.0 [M+H⁺].

1H NMR (400 MHz, DMSO-d6) δ ppm 11.16 (br s, 1H) 10.09 (s, 1H) 8.84 (d, 1H) 8.07 (dd, 1H) 7.75 (d, 1H) 7.30 (dd, 6.78 Hz, 1H) 7.10-7.19 (m, 2H).

TABLE 3
The compounds in Table 3 were prepared using the above procedure.

| | | | | LC-MS, |
|---|---|---|---|---|
| Ex- | Com- | | | MS-ESI, |
| ample | pound | | IUPAC | — |
| # | # | Final compound | Name | [M + H⁺]. |
| 32 | 144 | 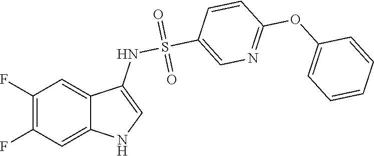 [SMILES: O=S(=O)(Nc1c[nH]c2cc(F)c(F)cc12)c1ccc(Oc2ccccc2)nc1] | N-(5,6- difluoro- 1H-indol-3- yl)-6- phenoxy- pyridine-3- sulfonamide | |
| 33 | 145 | 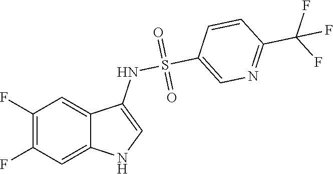 [SMILES: O=S(=O)(Nc1c[nH]c2cc(F)c(F)cc12)c1ccc(C(F)(F)F)nc1] | N-(5,6- difluoro- 1H-indol-3- yl)-6- (trifluoromethyl) pyridine-3- sulfonamide | |
| 34 | 146 | 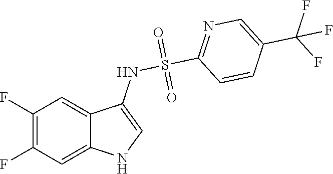 [SMILES: O=S(=O)(Nc1c[nH]c2cc(F)c(F)cc12)c1ccc(C(F)(F)F)cn1] | N-(5,6- difluoro- 1H-indol-3- yl)-5- (trifluoromethyl) pyridine-2- sulfonamide | |
| 35 | 147 | 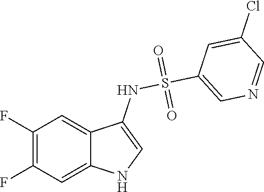 [SMILES: O=S(=O)(Nc1c[nH]c2cc(F)c(F)cc12)c1cncc(Cl)c1] | 5-chloro-N- (5,6- difluoro- 1H-indol-3- yl)pyridine-3- sulfonamide | |

### Biological Assays

STING pathway activation by the compounds described herein was measured using THP1-Dual™ cells (KO-IFNAR2).

THP1-Dual™ KO-IFNAR2 Cells (obtained from invivogen) were maintained in RPMI, 10% FCS, 5 ml P/S, 2 mM L-glut, 10 mM Hepes, and 1 mM sodium pyruvate. Compounds were spotted in empty 384 well tissue culture plates (Greiner 781182) by Echo for a final concentration of 0.0017-100 μM. Cells were plated into the TC plates at L per well, 2×10E6 cells/mL. For activation with STING ligand, 2′3′cGAMP (MW 718.38, obtained from Invivogen), was prepared in Optimem media.

The following solutions were prepared for each 1×384 plate:
o Solution A: 2 mL Optimem with one of the following stimuli:
   60 uL of 10 mM 2′3′cGAMP->150 μM stock
Solution B: 2 mL Optimem with 60 μL Lipofectamine 2000->Incubate 5 min at RT

2 mL of solution A and 2 ml Solution B was mixed and incubated for 20 min at room temperature (RT). 20 μL of transfection solution (A+B) was added on top of the plated cells, with a final 2′3′cGAMP concentration of 15 μM. The plates were then centrifuged immediately at 340 g for 1 minute, after which they were incubated at 37° C., 5% CO₂, >98% humidity for 24 h. Luciferase reporter activity was then measured. EC₅₀ values were calculated by using standard methods known in the art.

Luciferase reporter assay: 10 μL of supernatant from the assay was transferred to white 384-plate with flat bottom and squared wells. One pouch of QUANTI-Luc™ Plus was dissolved in 25 mL of water. 100 μL of QLC Stabilizer per 25 mL of QUANTI-Luc™ Plus solution was added. 50 μL of QUANTI-Luc™ Plus/QLC solution per well was then added. Luminescence was measured on a Platereader (e.g., Spectramax I3X (Molecular Devices GF3637001)).

Luciferase reporter activity was then measured. EC₅₀ values were calculated by using standard methods known in the art.

Table BA shows the activity of compounds in STING reporter assay: <0.008 μM=“++++++”; ≥0.008 and <0.04 μM=“+++++”; ≥0.04 and <0.2 μM=“++++”; ≥0.2 and <1 μM=“+++”; ≥1 and <5 μM=“++”; ≥5 and <100 μM=“+”.

TABLE BA

| | Human STING |
|---|---|
| | Reporter Assay |
| Compound # | EC₅₀ (μM) |
| 106 | + |
| 112 | >100.00 |
| 128 | ++ |
| 131 | + |
| 135 | + |
| 139 | + |
| 141 | + |

What is claimed is:

1. A method for inhibiting STING activity, the method comprising contacting STING with a compound of Formula I: 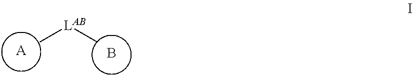
or a pharmaceutically acceptable salt thereof or a tautomer thereof,
wherein:
L^{AB} is —N(R^{N})S(O)₂—*, —N(R^{N})S(O)₂—(W^{AB1}—W^{AB2}—W^{AB3})—*, —S(O)₂N(R^{N})—*, wherein the asterisk represents point of attachment to B;
W^{AB1} is C₁₋₃ alkylene optionally substituted with from 1-4 independently selected R^{a};
W^{AB2} is a bond, —O—, —NR^{N}, or —S—;
W^{AB3} is a bond or C₁₋₃ alkylene optionally substituted with from 1-4 independently selected R^{a};
A is selected from the group consisting of:
(i) heteroaryl including from 5-6 ring atoms, wherein from 1-4 ring atoms are heteroatoms, each independently selected from the group consisting of N, N(H), N(R¹), N(R²), O, and S, and wherein from 1-5 ring atoms are carbon atoms, each independently selected from the group consisting of C, CH, CR¹, and CR³; provided that at least one ring atom is substituted with R¹; and
(ii) heteroaryl including from 7-20 ring atoms, wherein from 1-4 ring atoms are heteroatoms, each independently selected from the group consisting of N, N(H), N(R¹), N(R²), O, and S(O)₀₋₂, and wherein from 3-19 ring atoms are carbon atoms, each independently selected from the group consisting of C, CH, CH₂, CR¹, CHR¹, C(R¹)₂, CR³, CHR³, and C(R³)₂;
B is:
(a) C₁₋₁₅ alkyl which is optionally substituted with from 1-6 R^{a};
(b) C₃₋₂₀ cycloalkyl, which is optionally substituted with from 1-4 R^{b};
(c) C₆₋₂₀ aryl optionally substituted with from 1-4 R^{c};
(d) heteroaryl including from 5-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
(e) heterocyclyl including from 3-16 ring atoms, wherein from 1-3 ring atoms are heteroatoms, each independently selected from the group consisting of N(H), N(R^{d}), O, and S(O)₀₋₂ and wherein the heterocyclyl ring is optionally substituted with from 1-4 independently selected R^{b};
R^{N} is:
(i) H, or
(ii) C₁₋₆ alkyl optionally substituted with from 1-3 R^{a},
R¹ is:
(i) —(U¹)_{q}—U², wherein:
q is 0 or 1;
U¹ is C₁₋₆ alkylene, which is optionally substituted with from 1-6 R^{a}; and
U² is:
(a) C₃₋₁₂ cycloalkyl, which is optionally substituted with from 1-4 R^{b},
(b) C₆₋₁₀ aryl, which is optionally substituted with from 1-4 R^{c};
(c) heteroaryl including from 5-20 ring atoms, wherein from 1-4 ring atoms are heteroatoms, each independently selected from the group consisting of N, N(H), N(R^{d}), O, S(O)₀₋₂, and wherein the heteroaryl ring is optionally substituted with from 1-4 independently selected R^{c}, or
(d) heterocyclyl including from 3-12 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},
OR
(ii) C₁₋₁₀ alkyl, which is optionally substituted with from 1-6 independently selected R^{a};
each occurrence of R² is independently selected from the group consisting of:
(i) C₁₋₆ alkyl, which is optionally substituted with from 1-2 independently selected R^{a};
(ii) C₃₋₆ cycloalkyl;
(iii) heterocyclyl including from 3-10 ring atoms, wherein from 1-3 ring atoms are heteroatoms, each independently selected from the group consisting of N, N(H), N(R^{d}), O, and S(O)₀₋₂.
(iv) —C(O)(C₁₋₄ alkyl);
(v) —C(O)O(C₁₋₄ alkyl);
(vi) —CON(R′)(R″);
(vii) —S(O)₁₋₂(NR′R″);
(viii) —S(O)₁₋₂(C₁₋₄ alkyl);
(ix) —OH; and
(x) C₁₋₄ alkoxy;
each occurrence of R³ is independently selected from the group consisting of halo, cyano, C₂₋₆ alkenyl, C₂₋₆ alkynyl, C₁₋₄ alkoxy, C₁₋₄ haloalkoxy, —S(O)₁₋₂(C₁₋₄ alkyl), —NR^{e}R^{f}, —OH, oxo, —S(O)₁₋₂(NR′R″), —C₁₋₄ thioalkoxy, —NO₂, —C(═O)(C₁₋₄ alkyl), —C(═O)O(C₁₋₄ alkyl), —C(═O)OH, and —C(═O)N(R′)(R″);
each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;
each occurrence of R^{b} is independently selected from the group consisting of: C₁₋₁₀ alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};
each occurrence of R^{c} is independently selected from the group consisting of:
(a) halo;
(b) cyano;
(c) C₁₋₁₅ alkyl which is optionally substituted with from 1-6 independently selected R^{a};
(d) C₂₋₆ alkenyl;
(e) C₂₋₆ alkynyl;
(g) C₁₋₄ alkoxy optionally substituted with from 1-3 independently selected R^{a};
(h) C₁₋₄ haloalkoxy;
(i) —S(O)₁₋₂(C₁₋₄ alkyl);
(j) —NR^{e}R^{f};
(k) —OH;
(l) —S(O)₁₋₂(NR′R″);
(m) —C₁₋₄ thioalkoxy;
(n) —NO₂;
(o) —C(═O)(C₁₋₄ alkyl);
(p) —C(═O)O(C₁₋₄ alkyl);
(q) —C(═O)OH;
(r) —C(═O)N(R′)(R″); and
(s) -L¹-L²-R^{h};
R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;
each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R′ and R″), which are each independently selected from the group consisting of N(R^{d}), NH, O, and S;
-L¹ is a bond or C₁₋₃ alkylene;
-L² is —O—, —N(H)—, —S—, or a bond;
R^{h} is selected from:
C₃₋₈ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂ wherein the heterocyclyl is optionally substituted with from 1-4 substituents independently selected from the group consisting of halo, C₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 substituents independently selected from the group consisting of halo, C₁₋₄ alkyl, and C₁₋₄ haloalkyl; and
C₆₋₁₀ aryl, which is optionally substituted with from 1-4 substituents independently selected from the group consisting of halo, C₁₋₄ alkyl, or C₁₋₄ haloalkyl; and
each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(R^{d}), O, and S.

2. The method of claim 1, wherein A is: heteroaryl including from 7-20 ring atoms, wherein from 1-4 ring atoms are heteroatoms, each independently selected from the group consisting of N, N(H), N(R¹), N(R²), O, and S(O)₀₋₂, and wherein from 3-19 ring atoms are carbon atoms, each independently selected from the group consisting of C, CH, CH₂, CR¹, CHR¹, C(R¹)₂, CR³, CHR³, and C(R³)₂.

3. The method of any one of claims 1-2, wherein A is: heteroaryl including from 8-12 ring atoms, wherein from 1-4 ring atoms are heteroatoms, each independently selected from the group consisting of N, N(H), N(R¹), N(R²), O, and S(O)₀₋₂, and wherein from 4-11 ring atoms are carbon atoms, each independently selected from the group consisting of C, CH, CH₂, CR¹, CHR¹, C(R¹)₂, CR³, CHR³, and C(R³)₂.

4. The method of any one of claim 1-3, wherein A is: heteroaryl including from 8-10 ring atoms, wherein from 1-4 ring atoms are heteroatoms, each independently selected from the group consisting of N, N(H), N(R¹), N(R²), O, and S(O)₀₋₂, and wherein from 4-9 ring atoms are carbon atoms, each independently selected from the group consisting of C, CH, CH₂, CR¹, CHR¹, C(R¹)₂, CR³, CHR³, and C(R³)₂.

5. The method of any one of claims 1-4, wherein A is: heteroaryl including from 8-9 ring atoms, wherein from 1-4 ring atoms are heteroatoms, each independently selected from the group consisting of N, N(H), N(R¹), N(R²), O, and S(O)₀₋₂, and wherein from 4-8 ring atoms are carbon atoms, each independently selected from the group consisting of C, CH, CH₂, CR¹, CHR¹, C(R¹)₂, CR³, CHR³, and C(R³)₂.

6. The method of any one of claims 1-5, wherein A is (A-1): 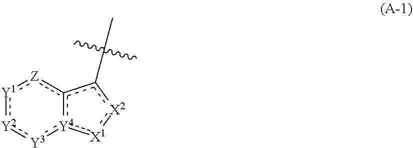
wherein
Z is selected from the group consisting of:
a bond, CH, CR¹, CR³, N, NH, N(R¹) and N(R²);
each of Y¹, Y², and Y³ is independently selected from the group consisting of O, S, CH, CR¹, CR³, N, NH, N(R¹), and NR²;
Y⁴ is C or N;
X¹ is selected from the group consisting of O, S, N, NH, NR¹, NR², CH, CR¹, and CR³;
X² is selected from the group consisting of O, S, N, NH, NR¹, NR², CH, CR¹, and CR³; and
each [symbol] is independently a single bond or a double bond, provided that the five-membered ring comprising Y⁴, X¹, and X² is heteroaryl; and the ring comprising Z, Y¹, Y², Y³, and Y⁴ is aromatic (i.e., carbocyclic aromatic or heteroaromatic).

7. The method of claim 6, wherein Z is selected from the group consisting of: CH, CR¹, CR³, N, and N(R²).

8. The method of any one of claims 6-7, wherein Z is selected from the group consisting of: CH, CR¹, CR³, and N.

9. The method of any one of claims 6-8, wherein Z is selected from the group consisting of CH, CR¹, and CR³ (e.g., Z is CH).

10. The method of any one of claims 6-9, wherein each of Y¹, Y², and Y³ is independently selected from the group consisting of CH, CR¹, CR³, and N.

11. The method of any one of claims 6-10, wherein each of Y¹, Y², and Y³ is independently selected from the group consisting of CH, CR¹, and CR³.

12. The method of any one of claims 6-11, wherein the 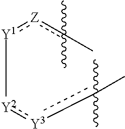
moiety is 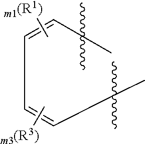
wherein m1=0, 1, 2, or 3; and m3=0, 1, 2, or 3 (e.g., m1=0 or 1; and m3=0, 1, or 2).

13. The method of any one of claims 6-10, wherein from 1-2 of Y¹, Y², and Y³ is independently N.

14. The method of any one of claims 6-10 and 13, wherein one of Y¹, Y², and Y³ is independently N.

15. The method of claim 14, wherein each of the remaining Y¹, Y², and Y³ is independently selected from the group consisting of CH, CR¹, and CR³.

16. The method of any one of claims 6-10 and 13-15, wherein the 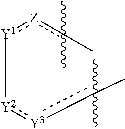
moiety is 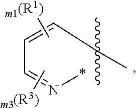
wherein:
the asterisk denotes point of attachment to Y⁴; and
m1=0, 1, or 2; and m3=0, 1, or 2 (e.g., m1=0 or 1; and m3=0 or 1).

17. The method of any one of claims 1-16, wherein Y⁴ is C.

18. The method of any one of claims 1-17, wherein X¹ is selected from the group consisting of O, S, NH, NR¹, and NR².

19. The method of any one of claims 1-18 wherein X¹ is selected from the group consisting of NH, NR¹, and NR² (e.g., X¹ can be NH).

20. The method of any one of claims 1-19, wherein X² is selected from the group consisting of N, CH, CR¹, and CR³.

21. The method of any one of claims 1-20, wherein X² is selected from the group consisting of N, C(C₁₋₃ alkyl), and CH.

22. The method of any one of claims 1-21, wherein X² is CH.

23. The method of any one of claims 1-22, X¹ and X², taken together, is 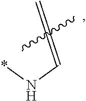 [SMILES: *NC=*]
wherein the asterisk denotes point of attachment to Y⁴.

24. The method of any one of 1-12 and 17-23, wherein A is: 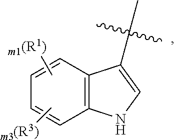
wherein m1=0, 1, 2, or 3; and m3=0, 1, 2, or 3 (e.g., m1=0 or 1; and m3=0, 1, or 2).

25. The method of any one of claims 1-10 and 13-16, wherein A is 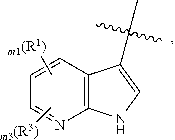
wherein m1=0, 1, or 2; and m3=0, 1, or 2 (e.g., m1=0 or 1; and m3=0 or 1).

26. The method of any one of claims 1-2, wherein A is (A-2): 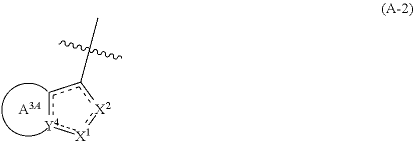
wherein
Ring A^{3A} is a monocyclic or bicyclic ring including from 5-12 ring atoms, wherein from 0-2 ring atoms are heteroatoms (including Y⁴ when Y⁴ is N), wherein each additional heteroatom is independently selected from the group consisting of N, N(H), N(R¹), N(R²), O, and S(O)₀₋₂, and from 3-12 ring atoms are ring carbon atoms each independently selected from C, CH, CH₂, CR¹, CHR¹, C(R¹)₂, CR³, CHR³, and C(R³)₂, provided that Ring A^{3A} is non-aromatic;
X¹ is selected from the group consisting of O, S, N, NH, NR¹, NR², CH, CR¹, and CR³;
X² is selected from the group consisting of O, S, N, NH, NR¹, NR², CH, CR¹, and CR³, provided that the ring including Y⁴, X¹, and X² is heteroaromatic; and
Y⁴ is selected from N or C.

27. The method of claim 26, wherein Y⁴ is N.

28. The method of claim 27, Ring A^{3A} is a monocyclic or bicyclic ring including from 5-11 ring atoms, wherein from 1-2 ring atoms are heteroatoms (including Y⁴), wherein the additional heteroatom is independently selected from the group consisting of N, N(H), N(R¹), N(R²), O, and S(O)₀₋₂, and from 3-11 ring atoms are ring carbon atoms each independently selected from C, CH, CH₂, CR¹, CHR¹, C(R¹)₂, CR³, CHR³, and C(R³)₂, provided that Ring A^{3A} is non-aromatic.

29. The method of claim 28, wherein Ring A^{3A} is a monocyclic or bicyclic ring including from 5-11 ring atoms, wherein 2 ring atoms are heteroatoms (including Y⁴), wherein the additional heteroatom is independently selected from the group consisting of N, N(H), N(R¹), N(R²), O, and S(O)₀₋₂, and from 3-11 ring atoms are ring carbon atoms each independently selected from C, CH, CH₂, CR¹, CHR¹, C(R¹)₂, CR³, CHR³, and C(R³)₂, provided that Ring A^{3A} is non-aromatic.

30. The method of claim 28, wherein Ring A^{3A} is a bicyclic (e.g., spirobicyclic ring) ring contains no additional heteroatoms in addition to Y⁴.

31. The method of claim 30, wherein A is: 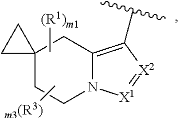
wherein m1=0, 1, or 2; and m3=0, 1, or 2 (e.g., m1=0 or 1; and m3=0 or 1).

32. The method of claim 29, wherein Ring A^{3A} is a monocyclic ring that contains an O atom.

33. The method of claim 32, wherein A is: 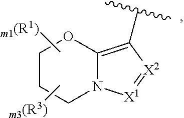
wherein m1=0, 1, or 2; and m3=0, 1, or 2 (e.g., m1=0 or 1; and m3=0 or 1).

34. The method of any one of claims 26-33, wherein X¹ is N.

35. The method of any one of claims 26-34, wherein X² is selected from CH and CR¹ (e.g., CH).

36. The method of claim 1, wherein A is heteroaryl including from 5-6 ring atoms, wherein from 1-4 ring atoms are heteroatoms, each independently selected from the group consisting of N, N(H), N(R¹), N(R²), O, and S, and wherein from 1-5 ring atoms are carbon atoms, each independently selected from the group consisting of C, CH, CR¹, and CR³; provided that at least one ring atom is substituted with R¹.

37. The method of claim 36, wherein A is heteroaryl including 5 ring atoms, wherein from 1-4 ring atoms are heteroatoms, each independently selected from the group consisting of N, N(H), N(R¹), N(R²), O, and S, and wherein from 1-4 ring atoms are carbon atoms, each independently selected from the group consisting of C, CH, CR¹, and CR³; provided that at least one ring atom is substituted with R¹.

38. The method of any one of claims 36-37, wherein A is heteroaryl including 5 ring atoms, wherein from 1-4 ring atoms are heteroatoms, each independently selected from the group consisting of N, N(H), N(R¹), N(R²), O, and S, and wherein from 1-4 ring atoms are carbon atoms, each independently selected from the group consisting of C, CH, CR¹, and CR³; provided that one ring atom is substituted with from one R¹.

39. The method of any one of claims 1 and 36-38, wherein A is (A-3): 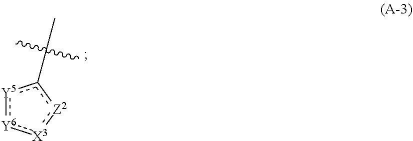
wherein:
Z² is selected from CH, CR², and N;
X³ is selected from O, S, N, NH, NR¹, NR², CH, CR¹, and CR³;
each of Y⁵ and Y⁶ is independently selected from O, S, CH, CR¹, CR³, NR², NH, and N; and
each [symbol] is independently a single bond or a double bond, provided that the five-membered ring comprising Y⁵, Y⁶, X³, and Z² is heteroaromatic.

40. The method of claim 39, wherein:
when X³ is NR¹ or CR¹, then each of Y⁵ and Y⁶ is independently selected from O, S, CH, CR³, NR², NH, and N; and
when X³ is selected from O, S, N, NH, NR², CH, and CR³, then one of Y⁵ and Y⁶ is CR¹ (in certain embodiments, the other of Y⁵ and Y⁶ is selected from O, S, CH, CR³, NR², NH, and N).

41. The method of any one of claims 39-40, wherein Z² is selected from CH and N.

42. The method of any one of claims 39-41, wherein Z² is CH.

43. The method of any one of claims 39-42, wherein Y⁶ is selected from N, CH, and CR³.

44. The method of any one of claims 39-43, wherein Y⁶ is N.

45. The method of any one of claims 39-44, wherein Y⁵ is CR¹.

46. The method of any one of claims 39-45, wherein X³ is selected from S, O, NH, and N(R²) (e.g., NH).

47. The method of any one of claims 39-46, wherein A is

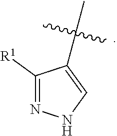

48. The method of any one of claims 1-47, wherein each occurrence of R¹ is independently selected from:
(i) —(U¹)_{q}—U², wherein:
q is 0 or 1;
U¹ is C₁₋₆ alkylene, which is optionally substituted with from 1-6 R^{a}; and
U² is:
(a) C₃₋₁₀ cycloalkyl, which is optionally substituted with from 1-4 R^{b},
(b) C₆₋₁₀ aryl, which is optionally substituted with from 1-4 R^{c};
(c) heteroaryl including from 5-10 ring atoms, wherein from 1-4 ring atoms are heteroatoms, each independently selected from the group consisting of N, N(H), N(R^{d}), O, S, and S(O)₂ and wherein the heteroaryl ring is optionally substituted with from 1-4 independently selected R^{c}, or
(d) heterocyclyl including from 3-10 ring atoms, wherein from 1-3 ring atoms are heteroatoms, each independently selected from the group consisting of N, N(H), N(R^{d}), and O, and wherein the heterocyclyl ring is optionally substituted with from 1-4 independently selected R^{b},
and
(ii) C₁₋₆ alkyl, which is optionally substituted with from 1-6 independently selected R^{a}.

49. The method of any one of claims 1-48, wherein R¹ is —(U¹)_{q}—U².

50. The method of any one of claims 1-49, wherein q is 0.

51. The method of any one of claims 1-50, wherein U² is C₆₋₁₀ aryl, which is optionally substituted with from 1-4 R^{c}.

52. The method of any one of claims 1-51, wherein U² is C₆₋₁₀ aryl, which is optionally substituted with from 1-2 R^{c}.

53. The method of any one of claims 1-52, wherein U² is phenyl, which is optionally substituted with from 1-2 (e.g., 1) R^{c}.

54. The method of any one of claims 51-53, wherein each occurrence of R^{c} substituent on U² is independently selected from: halo, cyano, C₁₋₆ alkyl, and C₁₋₄ haloalkyl.

55. The method of any one of claims 51-54, wherein each occurrence of R^{c} substituent on U² is independently selected from halo.

56. The method of any one of claims 1-55, wherein R¹ is phenyl, which is optionally substituted with from 1-2 (e.g., 0; e.g., 1) R^{c}.

57. The method of claim 56, wherein each R^{c} is as defined in any one of claims 54-55.

58. The method of any one of claims 1-57, wherein each occurrence of R³ is independently selected from the group consisting of: halo, cyano, C₁₋₄ alkoxy, C₁₋₄ haloalkoxy, —S(O)₁₋₂(C₁₋₄ alkyl), —NR^{e}R^{f}, —OH, —S(O)₁₋₂(NR′R″), —C₁₋₄ thioalkoxy, —C(═O)(C₁₋₄ alkyl), —C(═O)O(C₁₋₄ alkyl), —C(═O)OH, and —C(═O)N(R′)(R″).

59. The method of any one of claims 1-58, wherein each occurrence of R³ is independently selected from the group consisting of: halo, cyano, C₁₋₄ alkoxy, C₁₋₄ haloalkoxy, —S(O)₁₋₂(C₁₋₄ alkyl), —S(O)₁₋₂(NR′R″), —C(═O)(C₁₋₄ alkyl), —C(═O)O(C₁₋₄ alkyl), —C(═O)OH, and —C(═O)N(R′)(R″).

60. The method of any one of claims 1-59, wherein each occurrence of R³ is independently selected from the group consisting of: halo, cyano, C₁₋₄ alkoxy, and C₁₋₄ haloalkoxy (e.g., R³ can be halo).

61. The method of any one of claims 1-60, wherein each occurrence of R² is independently selected from
(i) C₁₋₆ alkyl (e.g., methyl);
(ii) C₃₋₆ cycloalkyl;
(iv) —C(O)(C₁₋₄ alkyl) (e.g., C(O)Me);
(v) —C(O)O(C₁₋₄ alkyl);
(vi) —CON(R′)(R″);
(vii) —S(O)₁₋₂(NR′R″); and
(viii) —S(O)₁₋₂(C₁₋₄ alkyl) (e.g., S(O)₂Me).

62. The method of any one of claims 12, 16, 24, 25, 31, and 33, wherein m1=1.

63. The method of claim 62, wherein m3=0.

64. The method of any one of claims 62-63, wherein R¹ is as defined in any one of claims 48-57.

65. The method of any one of claims 12, 16, 24, 25, 31, and 33, wherein m1=0.

66. The method of claim 65, wherein m3=0.

67. The method of claim 65, wherein m3=1 or 2 (e.g., 1).

68. The method of claim 67, wherein each occurrence of R³ is as defined in any one of claims 58-60.

69. The method of claim 68, wherein each occurrence of R³ is independently halo (e.g., F).

70. The method of any one of claims 1-69, wherein B is phenyl substituted with from 1-4 R^{c}.

71. The method of any one of claims 1-70, wherein B is phenyl substituted with from 1-2 R^{c}, wherein one R^{c} is at the ring carbon para to the point of attachment to the L^{AB} moiety in Formula I.

72. The method of any one of claims 1-71, wherein B is phenyl substituted with one R^{c} which is at the ring carbon para to the point of attachment to the L^{AB} moiety in Formula I.

73. The method of any one of claims 70-72, wherein each occurrence of R^{c} substituent on B is independently selected from:
(a) halo;
(b) cyano;
(c) C₁₋₁₀ alkyl which is optionally substituted with from 1-6 independently selected R^{a};
(g) C₁₋₄ alkoxy;
(h) C₁₋₄ haloalkoxy;
(i) —S(O)₁₋₂(C₁₋₄ alkyl);
(m) —C₁₋₄ thioalkoxy;
(o) —C(═O)(C₁₋₄ alkyl);
(p) —C(═O)O(C₁₋₄ alkyl);
(r) —C(═O)N(R′)(R″); and
(s) -L¹-L²-R^{h}.

74. The method of any one of claims 70-73, wherein each occurrence of R^{c} substituent on B is independently selected from:
(a) halo;
(b) cyano;
(c) C₁₋₁₀ alkyl which is optionally substituted with from 1-6 independently selected R^{a};
(g) C₁₋₄ alkoxy;
(h) C₁₋₄ haloalkoxy; and
(s) -L¹-L²-R^{h}.

75. The method of any one of claims 70-74, wherein each occurrence of R^{c} substituent on B is independently selected from:
(a) halo;
(c) C₁₋₁₀ alkyl which is optionally substituted with from 1-6 independently selected R^{a}; and
(s) -L¹-L²-R^{h}.

76. The method of any one of claims 70-75, wherein one occurrence of R^{c} is C₁₋₁₀ alkyl which is optionally substituted with from 1-6 independently selected R^{a}.

77. The method of any one of claims 70-76, wherein one occurrence of R^{c} is C₁₋₆ alkyl which is optionally substituted with from 1-6 independently selected R^{a}.

78. The method of any one of claims 70-77, wherein one occurrence of R^{c} is unsubstituted C₁₋₁₀ alkyl.

79. The method of claim 78, wherein one occurrence of R^{c} is unsubstituted C₂₋₁₀ (e.g., C₂₋₃, e.g., C₃₋₄, e.g., C₄₋₁₀) alkyl.

80. The method of any one of claims 70-77, wherein one occurrence of R^{c} is C₁₋₆ alkyl which is substituted with from 1-6 independently selected R^{a}.

81. The method of any one of claims 70-77 and 80, wherein one occurrence of R^{c} is CF₃ or 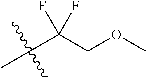 [SMILES: *C(F)(F)COC]
(e.g., R^{c} can be CF₃).

82. The method of any one of claims 76-81, wherein a second occurrence of R^{c} when present is independently halo.

83. The method of any one of claims 76-81, wherein B is phenyl substituted with from 1-3 occurrences of R^{c}; and one occurrence of R^{c} is at the ring carbon para to the point of attachment to the L^{AB} moiety in Formula I.

84. The method of any one of claims 1-69 and 83, wherein B is 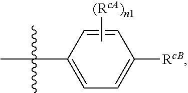
wherein: n1=0 or 1; and each of R^{cA} and R^{cB} is an independently selected R^{c}.

85. The method of claim 84, wherein R^{cB} is R^{c} that is as defined in any one of claims 76-82.

86. The method of claim 84, wherein R^{cB} is R^{c} that is as defined in any one of claims 78-79.

87. The method of claim 85, wherein R^{cB} is R^{c} that is as defined in any one of claims 80-81.

88. The method of any one of claims 84-87, wherein n1 is 0.

89. The method of any one of claims 84-87, wherein n1 is 1; and R^{cA} is halo.

90. The method of any one of claims 1-89, wherein L^{AB} is —N(R^{N})S(O)₂—*.

91. The method of any one of claims 1-89, wherein L^{AB} is —N(R^{N})S(O)₂—(W^{AB1}—W^{AB2}—W^{AB3})—*, such as —N(R^{N})S(O)₂—(C₁₋₃ alkylene)- or —N(R^{N})S(O)₂—(C₁₋₃ alkylene)-O—(C₁₋₃ alkylene).

92. The method of any one of claims 1-91, wherein R^{N} is H.

93. The method of claim 1, wherein the compound has Formula (I-1): 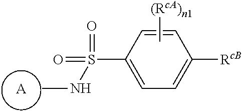
wherein n1=0 or 1; and each of R^{cA} and R^{cB} is an independently selected R^{c}.

94. The method of claim 1, wherein the compound has Formula (I-2): 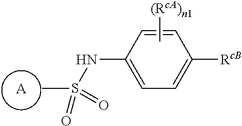
wherein n1=0 or 1; and
each of R^{cA} and R^{cB} is an independently selected R^{c}.

95. The method of claims 93-94, wherein A is (A-1) as defined in claim 6.

96. The method of any one of claims 93-95, wherein A is as defined in claim 24.

97. The method of any one of claims 93-95, wherein A is as defined in claim 25.

98. The method of any one of claims 96-97, wherein m1=0.

99. The method of claim 98, wherein m3=1.

100. The method of claim 99, wherein R³ is as defined in any one of claims 48-50.

101. The method of claim 98, wherein m3=0.

102. The method of any one of claims 93-94, wherein A is (A-2) as defined in claim 26.

103. The method of any one of claims 93-94 and 102, wherein A is as defined in any one of claims 30-31 (e.g., claim 31).

104. The method of any one of claims 93-94 and 102, wherein A is as defined in any one of claims 32-33 (e.g., claim 33).

105. The method of any one of claims 103-104, wherein m1=0.

106. The method of any one of claims 103-104, wherein m3=0.

107. The method of any one of claims 93-94, wherein A is (A-3) as defined in claim 39.

108. The method of claim 107, wherein A is as defined in claim 47.

109. The method of claim any one of claims 107-108, wherein R¹ is as defined in any one of claims 56-57.

110. The method of any one of claims 93-109, wherein R^{cB} is R^{c} that is as defined in any one of claims 76-82.

111. The method of any one of claims 93-109, wherein R^{cB} is R^{c} that is as defined in any one of claims 78-79.

112. The method of any one of claims 93-109, wherein R^{cB} is R^{c} that is as defined in any one of claims 80-81.

113. The method of any one of claims 93-112, wherein n1 is 0.

114. The method of any one of claims 93-112, wherein n1 is 1; and R^{cA} is halo.

115. The method of claim 1, wherein A is selected from the group consisting of: 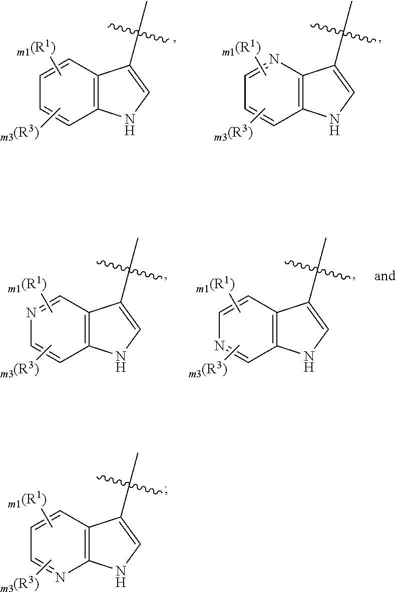
m1 is 0 or 1; and m3 is 0, 1, or 2;
L^{AB} is —N(H)S(O)₂—* and —NHS(O)₂—(W^{AB1})*; and
B is selected from the group consisting of:
C₆ aryl substituted with from 1-4 R^{c};
heteroaryl including from 5-6 ring atoms, wherein from 1-3 ring atoms are heteroatoms, each independently selected from the group consisting of N, N(H), N(R^{d}), O, and S(O)₀₋₂, and wherein the heteroaryl ring is substituted with from 1-4 independently selected R^{c};
bicyclic or tricyclic heteroaryl including from 9-15 ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c};
C₅₋₁₅ alkyl which is optionally substituted with from 1-6 R^{a}.
C₆₋₂₀ aryl optionally substituted with from 1-4 R^{c}; and
heteroaryl including from 7-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}.

116. The method of claim 1, wherein the compound is selected from the compounds in Table C1; or a pharmaceutically acceptable salt thereof.

117. The method of claim 1, wherein the method comprising administering a pharmaceutical composition comprising a compound of claims 1-116 and one or more pharmaceutically acceptable excipients.

118. The method of any one of claims 1-116, wherein the inhibiting comprises antagonizing STING.

119. The method of any one of claims 1-116 and 118, which is carried out in vitro.

120. The method of claim 119, wherein the method comprises contacting a sample comprising one or more cells comprising STING with the compound.

121. The method of claim 120, wherein the one or more cells are one or more cancer cells.

122. The method of claim 120 or 121 wherein the sample further comprises one or more cancer cells (e.g., wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma).

123. The method of any one of claims 1-116 and 118, which is carried out in vivo.

124. The method of claim 123, wherein the method comprises administering the compound to a subject having a disease in which increased (e.g., excessive) STING signaling contributes to the pathology and/or symptoms and/or progression of the disease.

125. The method of claim 124, wherein the subject is a human.

126. The method of claim 124, wherein the disease is cancer.

127. The method of claim 126, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

128. The method of claim 126 or 127, wherein the cancer is a refractory cancer.

129. The method of claim 124, wherein the compound is administered in combination with one or more additional cancer therapies.

130. The method of claim 129, wherein the one or more additional cancer therapies comprises surgery, radiotherapy, chemotherapy, toxin therapy, immunotherapy, cryotherapy or gene therapy, or a combination thereof.

131. The method of claim 130, wherein chemotherapy comprises administering one or more additional chemotherapeutic agents.

132. The method of claim 131, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

133. The method of any one of claims 124-132, wherein the compound is administered intratumorally.

134. A method of treating cancer, comprising administering to a subject in need of such treatment an effective amount of a compound as claimed in any one of claims 1-116, or a pharmaceutical composition as claimed in claim 117.

135. The method of claim 134, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

136. The method of claim 134 or 135, wherein the cancer is a refractory cancer.

137. The method of claim 134, wherein the compound is administered in combination with one or more additional cancer therapies.

138. The method of claim 137, wherein the one or more additional cancer therapies comprises surgery, radiotherapy, chemotherapy, toxin therapy, immunotherapy, cryotherapy or gene therapy, or a combination thereof.

139. The method of claim 138, wherein chemotherapy comprises administering one or more additional chemotherapeutic agents.

140. The method of claim 139, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

141. The method of any one of claims 134-140, wherein the compound is administered intratumorally.

142. A method of inducing an immune response in a subject in need thereof, the method comprising administering to the subject an effective amount of a compound as claimed in any one of claims 1-116, or a pharmaceutical composition as claimed in claim 117.

143. The method of claim 142, wherein the subject has cancer.

144. The method of claim 143, wherein the subject has undergone and/or is undergoing and/or will undergo one or more cancer therapies.

145. The method of claim 143, wherein the cancer selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

146. The method of claim 145, wherein the cancer is a refractory cancer.

147. The method of claim 142, wherein the immune response is an innate immune response.

148. The method of claim 147, wherein the at least one or more cancer therapies comprises surgery, radiotherapy, chemotherapy, toxin therapy, immunotherapy, cryotherapy or gene therapy, or a combination thereof.

149. The method of claim 148, wherein chemotherapy comprises administering one or more additional chemotherapeutic agents.

150. The method of claim 149, wherein the one or more additional chemotherapeutic agents is selected from alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

151. A method of treatment of a disease in which increased (e.g., excessive) STING signaling contributes to the pathology and/or symptoms and/or progression of the disease, comprising administering to a subject in need of such treatment an effective amount of a compound as claimed in any one of claims 1-116, or a pharmaceutical composition as claimed in claim 117.

152. A method of treatment comprising administering to a subject having a disease in which increased (e.g., excessive) STING signaling contributes to the pathology and/or symptoms and/or progression of the disease an effective amount of a compound as claimed in any one of claims 1-116, or a pharmaceutical composition as claimed in claim 117.

153. A method of treatment comprising administering to a subject a compound as claimed in any one of claims 1-116, or a pharmaceutical composition as claimed in claim 117, wherein the compound or composition is administered in an amount effective to treat a disease in which increased (e.g., excessive) STING signaling contributes to the pathology and/or symptoms and/or progression of the disease, thereby treating the disease.

154. The method of any one of claims 151-153, wherein the disease is cancer.

155. The method of claim 154, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

156. The method of claim 154 or 155, wherein the cancer is a refractory cancer.

157. The method of any one of claims 154-156, wherein the compound is administered in combination with one or more additional cancer therapies.

158. The method of claim 157, wherein the one or more additional cancer therapies comprises surgery, radiotherapy, chemotherapy, toxin therapy, immunotherapy, cryotherapy or gene therapy, or a combination thereof.

159. The method of claim 158, wherein chemotherapy comprises administering one or more additional chemotherapeutic agents.

160. The method of claim 159, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

161. The method of any one of claims 151-160, wherein the compound is administered intratumorally.

162. A method of treatment of a disease, disorder, or condition associated with STING, comprising administering to a subject in need of such treatment an effective amount of a compound as claimed in any one of claims 1-116, or a pharmaceutical composition as claimed in claim 117.

163. The method of claim 162, wherein the disease, disorder, or condition is selected from type I interferonopathies, Aicardi-Goutières Syndrome (AGS), genetic forms of lupus, inflammation-associated disorders, and rheumatoid arthritis.

164. The method of claim 163, wherein the disease, disorder, or condition is a type I interferonopathy (e.g., STING-associated vasculopathy with onset in infancy (SAVI)).

165. The method of claim 164, wherein the type I interferonopathy is STING-associated vasculopathy with onset in infancy (SAVI)).

166. The method of claim 163, wherein the disease, disorder, or condition is Aicardi-Goutières Syndrome (AGS).

167. The method of claim 163, wherein the disease, disorder, or condition is a genetic form of lupus.

168. The method of claim 163, wherein the disease, disorder, or condition is inflammation-associated disorder.

169. The method of claim 168, wherein the inflammation-associated disorder is systemic lupus erythematosus.

170. The method of claim 163, wherein the disease, disorder, or condition is rheumatoid arthritis.

171. The method of any one of claims 118-170, wherein the method further comprises identifying the subject.

172. A compound of Formula I: 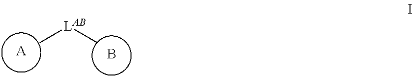
or a pharmaceutically acceptable salt thereof or a tautomer thereof,
wherein:
L^{AB} is —N(R^{N})S(O)₂—*, —N(R^{N})S(O)₂—(W^{AB1}—W^{AB2}—W^{AB3})—*, —S(O)₂N(R^{N})—*, wherein the asterisk represents point of attachment to B;
W^{AB1} is C₁₋₃ alkylene optionally substituted with from 1-4 independently selected R^{a};
W^{AB2} is a bond, —O—, —NR^{N}, or —S—;
W^{AB3} is a bond or C₁₋₃ alkylene optionally substituted with from 1-4 independently selected R^{a};
A is selected from the group consisting of:
(i) heteroaryl including from 5-6 ring atoms, wherein from 1-4 ring atoms are heteroatoms, each independently selected from the group consisting of N, N(H), N(R¹), N(R²), O, and S, and wherein from 1-5 ring atoms are carbon atoms, each independently selected from the group consisting of C, CH, CR¹, and CR³; provided that at least one ring atom is substituted with R¹; and
(ii) heteroaryl including from 7-20 ring atoms, wherein from 1-4 ring atoms are heteroatoms, each independently selected from the group consisting of N, N(H), N(R¹), N(R²), O, and S(O)₀₋₂, and wherein from 3-19 ring atoms are carbon atoms, each independently selected from the group consisting of C, CH, CH₂, CR¹, CHR¹, C(R¹)₂, CR³, CHR³, and C(R³)₂;
B is:
(a) C₁₋₁₅ alkyl which is optionally substituted with from 1-6 R^{a};
(b) C₃₋₂₀ cycloalkyl, which is optionally substituted with from 1-4 R^{b};
(c) C₆₋₂₀ aryl optionally substituted with from 1-4 R^{c};
(d) heteroaryl including from 5-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
(e) heterocyclyl including from 3-16 ring atoms, wherein from 1-3 ring atoms are heteroatoms, each independently selected from the group consisting of N(H), N(R^{d}), O, and S(O)₀₋₂ and wherein the heterocyclyl ring is optionally substituted with from 1-4 independently selected R^{b};
R^{N} is:
(i) H, or
(ii) C₁₋₆ alkyl optionally substituted with from 1-3 R^{a},
R¹ is:
(i) —(U¹)_{q}—U², wherein:
q is 0 or 1;
U¹ is C₁₋₆ alkylene, which is optionally substituted with from 1-6 R^{a}; and
U² is:
(a) C₃₋₁₂ cycloalkyl, which is optionally substituted with from 1-4 R^{b},
(b) C₆₋₁₀ aryl, which is optionally substituted with from 1-4 R^{c};
(c) heteroaryl including from 5-20 ring atoms, wherein from 1-4 ring atoms are heteroatoms, each independently selected from the group consisting of N, N(H), N(R^{d}), O, S(O)₀₋₂, and wherein the heteroaryl ring is optionally substituted with from 1-4 independently selected R^{c}, or
(d) heterocyclyl including from 3-12 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},
OR
(ii) C₁₋₁₀ alkyl, which is optionally substituted with from 1-6 independently selected R^{a};
each occurrence of R² is independently selected from the group consisting of:
(i) C₁₋₆ alkyl, which is optionally substituted with from 1-2 independently selected R^{a};
(ii) C₃₋₆ cycloalkyl;
(iii) heterocyclyl including from 3-10 ring atoms, wherein from 1-3 ring atoms are heteroatoms, each independently selected from the group consisting of N, N(H), N(R^{d}), O, and S(O)₀₋₂.
(iv) —C(O)(C₁₋₄ alkyl);
(v) —C(O)O(C₁₋₄ alkyl);
(vi) —CON(R′)(R″);
(vii) —S(O)₁₋₂(NR′R″);
(viii) —S(O)₁₋₂(C₁₋₄ alkyl);
(ix) —OH; and
(x) C₁₋₄ alkoxy;
each occurrence of R³ is independently selected from the group consisting of halo, cyano, C₂₋₆ alkenyl, C₂₋₆ alkynyl, C₁₋₄ alkoxy, C₁₋₄ haloalkoxy, —S(O)₁₋₂(C₁₋₄ alkyl), —NR^{e}R^{f}, —OH, oxo, —S(O)₁₋₂(NR′R″), —C₁₋₄ thioalkoxy, —NO₂, —C(═O)(C₁₋₄ alkyl), —C(═O)O(C₁₋₄ alkyl), —C(═O)OH, and —C(═O)N(R′)(R″);
each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;
each occurrence of R^{b} is independently selected from the group consisting of: C₁₋₁₀ alkyl optionally substituted with from 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₄ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};
each occurrence of R^{c} is independently selected from the group consisting of:
(a) halo;
(b) cyano;
(c) C₁₋₁₅ alkyl which is optionally substituted with from 1-6 independently selected R^{a};
(d) C₂₋₆ alkenyl;
(e) C₂₋₆ alkynyl;
(g) C₁₋₄ alkoxy optionally substituted with from 1-3 independently selected R^{a};
(h) C₁₋₄ haloalkoxy;
(i) —S(O)₁₋₂(C₁₋₄ alkyl);
(j) —NR^{e}R^{f};
(k) —OH;
(l) —S(O)₁₋₂(NR′R″);
(m) —C₁₋₄ thioalkoxy;
(n) —NO₂;
(o) —C(═O)(C₁₋₄ alkyl);
(p) —C(═O)O(C₁₋₄ alkyl);
(q) —C(═O)OH;
(r) —C(═O)N(R′)(R″); and
(s) -L¹-L²-R^{h};
R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;
each occurrence of R^{e} and R^{f} is independently selected from the group consisting of: H; C₁₋₆ alkyl; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R′ and R″), which are each independently selected from the group consisting of N(R^{d}), NH, O, and S;
-L¹ is a bond or C₁₋₃ alkylene;
-L² is —O—, —N(H)—, —S—, or a bond;
R^{h} is selected from:
C₃₋₈ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂ wherein the heterocyclyl is optionally substituted with from 1-4 substituents independently selected from the group consisting of halo, C₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 substituents independently selected from the group consisting of halo, C₁₋₄ alkyl, and C₁₋₄ haloalkyl; and
C₆₋₁₀ aryl, which is optionally substituted with from 1-4 substituents independently selected from the group consisting of halo, C₁₋₄ alkyl, or C₁₋₄ haloalkyl; and
each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(R^{d}), O, and S.

173. The compound of claim 172, wherein A is (A-1): 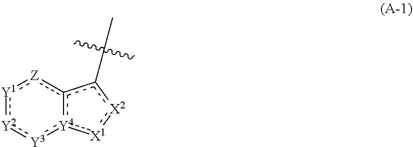
wherein
Z is selected from the group consisting of:
a bond, CH, CR¹, CR³, N, NH, N(R¹) and N(R²);
each of Y¹, Y², and Y³ is independently selected from the group consisting of O, S, CH, CR¹, CR³, N, NH, N(R¹), and NR²;
Y⁴ is C or N;
X¹ is selected from the group consisting of O, S, N, NH, NR¹, NR², CH, CR¹, and CR³;
X² is selected from the group consisting of O, S, N, NH, NR¹, NR², CH, CR¹, and CR³; and
each [symbol] is independently a single bond or a double bond, provided that the five-membered ring comprising Y⁴, X¹, and X² is heteroaryl; and the ring comprising Z, Y¹, Y², Y³, and Y⁴ is aromatic (i.e., carbocyclic aromatic or heteroaromatic).

174. The compound of claim 173, wherein the 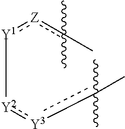
moiety is 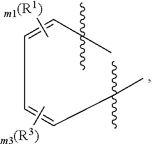
wherein m1=0, 1, 2, or 3; and m3=0, 1, 2, or 3 (e.g., m1=0 or 1; and m3=0, 1, or 2).

175. The compound of claim 173, wherein from 1-2 of Y¹, Y², and Y³ is independently N.

176. The compound of claim 175, wherein the 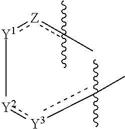
moiety is 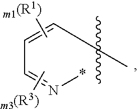
wherein the asterisk denotes point of attachment to Y⁴; an m1=0, 1, or 2; and m3=0, 1, or 2 (e.g., m1=0 or 1; and m3=0 or 1).

177. The compound of any one of claims 173-176, wherein Y⁴ is C.

178. The compound of any one of claims 173-177, wherein X¹ is selected from the group consisting of NH, NR¹, and NR², such as X¹ is NH.

179. The compound of any one of claims 173-178, wherein X² is selected from the group consisting of N, C(C₁₋₃ alkyl), and CH.

180. The compound of any one of claims 173-179, wherein X² is CH.

181. The compound of any one of claims 172-174, wherein A is: 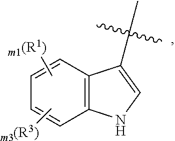
wherein m1=0, 1, 2, or 3; and m3=0, 1, 2, or 3 (e.g., m1=0 or 1; and m3=0, 1, or 2).

182. The compound of any one of claims 172-173 and 175-176, wherein A is 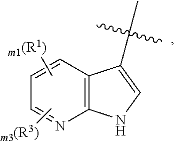
wherein m1=0, 1, or 2; and m3=0, 1, or 2 (e.g., m1=0 or 1; and m3=0 or 1).

183. The compound of claim 172, wherein A is (A-2): 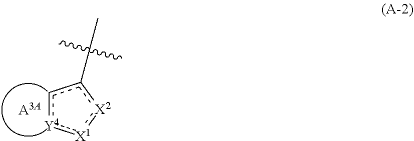
wherein
Ring A^{3A} is a monocyclic or bicyclic ring including from 5-12 ring atoms, wherein from 0-2 ring atoms are heteroatoms (including Y⁴ when Y⁴ is N), wherein each additional heteroatom is independently selected from the group consisting of N, N(H), N(R¹), N(R²), O, and S(O)₀₋₂, and from 3-12 ring atoms are ring carbon atoms each independently selected from C, CH, CH₂, CR¹, CHR¹, C(R¹)₂, CR³, CHR³, and C(R³)₂, provided that Ring A^{3A} is non-aromatic;
X¹ is selected from the group consisting of O, S, N, NH, NR¹, NR², CH, CR¹, and CR³;
X² is selected from the group consisting of O, S, N, NH, NR¹, NR², CH, CR¹, and CR³, provided that the ring including Y⁴, X¹, and X² is heteroaromatic; and
Y⁴ is selected from N or C.

184. The compound of claim 183, wherein Y⁴ is N.

185. The compound of any one of claims 183-184, wherein A is: 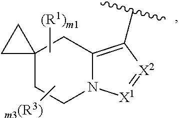
wherein m1=0, 1, or 2; and m3=0, 1, or 2 (e.g., m1=0 or 1; and m3=0 or 1).

186. The compound of any one of claims 183-184, wherein A is: 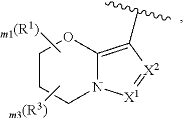
wherein m1=0, 1, or 2; and m3=0, 1, or 2 (e.g., m1=0 or 1; and m3=0 or 1).

187. The compound of any one of claim 172, wherein A is (A-3): 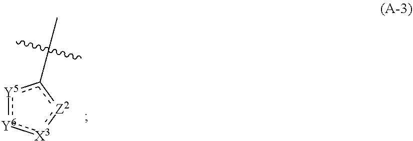
wherein:
Z² is selected from CH, CR², and N;
X³ is selected from O, S, N, NH, NR¹, NR², CH, CR¹, and CR³;
each of Y⁵ and Y⁶ is independently selected from O, S, CH, CR¹, CR³, NR², NH, and N; and
each [symbol] is independently a single bond or a double bond, provided that the five-membered ring comprising Y⁵, Y⁶, X³, and Z² is heteroaromatic.

188. The compound of any one of claims 172 and 187, wherein A is

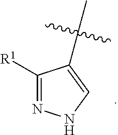

189. The compound of any one of claims 172-188, wherein each occurrence of R¹ is independently selected from the group consisting of:
(i) —(U¹)_{q}—U², wherein:
q is 0 or 1;
U¹ is C₁₋₆ alkylene, which is optionally substituted with from 1-6 R^{a}; and
U² is:
C₃₋₁₀ cycloalkyl, which is optionally substituted with from 1-4 R^{b},
C₆₋₁₀ aryl, which is optionally substituted with from 1-4 R^{c};
heteroaryl including from 5-10 ring atoms, wherein from 1-4 ring atoms are heteroatoms, each independently selected from the group consisting of N, N(H), N(R^{d}), O, S, and S(O)₂ and wherein the heteroaryl ring is optionally substituted with from 1-4 independently selected R^{c}, or
heterocyclyl including from 3-10 ring atoms, wherein from 1-3 ring atoms are heteroatoms, each independently selected from the group consisting of N, N(H), N(R^{d}), and O, and wherein the heterocyclyl ring is optionally substituted with from 1-4 independently selected R^{b}, and
(ii) C₁₋₆ alkyl, which is optionally substituted with from 1-6 independently selected R^{a}.

190. The compound of any one of claims 172-189, wherein R¹ is —(U¹)_{q}—U².

191. The compound of any one of claims 172-190, wherein q is 0.

192. The compound of any one of claims 172-191, wherein U² is phenyl, which is optionally substituted with from 1-2 (e.g., 1) R^{c}.

193. The compound of any one of claims 190-192, wherein each occurrence of R^{c} substituent on U² is independently selected from: halo, cyano, C₁₋₆ alkyl, and C₁₋₄ haloalkyl.

194. The compound of any one of claims 172-193, wherein each occurrence of R³ is independently selected from the group consisting of: halo, cyano, C₁₋₄ alkoxy, C₁₋₄ haloalkoxy, —S(O)₁₋₂(C₁₋₄ alkyl), —NR^{e}R^{f}, —OH, —S(O)₁₋₂(NR′R″), —C₁₋₄ thioalkoxy, —C(═O)(C₁₋₄ alkyl), —C(═O)O(C₁₋₄ alkyl), —C(═O)OH, and —C(═O)N(R′)(R″).

195. The compound of any one of claims 172-194, wherein each occurrence of R² is independently selected from
(ii) C₁₋₆ alkyl (e.g., methyl);
(iii) C₃₋₆ cycloalkyl;
(iv) —C(O)(C₁₋₄ alkyl) (e.g., C(O)Me);
(v) —C(O)O(C₁₋₄ alkyl);
(vi) —CON(R′)(R″);
(vii) —S(O)₁₋₂(NR′R″); and
(viii) —S(O)₁₋₂(C₁₋₄ alkyl) (e.g., S(O)₂Me).

196. The compound of any one of claims 174, 176, 181, 182, 185, and 186, wherein m1=1.

197. The compound of claim 196, wherein m3=0.

198. The compound of any one of claims 174, 176, 181, 182, 185, and 186, wherein m3=1 or 2; and m1=0.

199. The compound of any one of claims 172-198, wherein B is phenyl substituted with from 1-4 R^{c}.

200. The compound of any one of claims 172-199, wherein B is phenyl substituted with from 1-2 R^{c}, wherein one R^{c} is at the ring carbon para to the point of attachment to the L^{AB} moiety in Formula I.

201. The compound of any one of claims 199-200, wherein each occurrence of R^{c} substituent on B is independently selected from: halo; cyano; C₁₋₁₀ alkyl which is optionally substituted with from 1-6 independently selected R^{a}; C₁₋₄ alkoxy; C₁₋₄ haloalkoxy; —S(O)₁₋₂(C₁₋₄ alkyl); —C₁₋₄ thioalkoxy; —C(═O)(C₁₋₄ alkyl); —C(═O)O(C₁₋₄ alkyl); —C(═O)N(R′)(R″); and -L¹-L²-R^{h}.

202. The compound of any one of claims 172-200, wherein B is 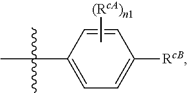
wherein: n1=0 or 1; and each of R^{cA} and R^{cB} is an independently selected R^{c}.

203. The compound of claim 202, wherein R^{cB} is C₁₋₁₀ alkyl which is optionally substituted with from 1-6 independently selected R^{a}, such as C₁₋₆ alkyl which is optionally substituted with from 1-6 independently selected R^{a};
optionally R^{cB} is unsubstituted C₁₋₁₀ alkyl, such as unsubstituted C₂₋₁₀ (e.g., C₂₋₃, e.g., C₃₋₄, e.g., C₄₋₁₀) alkyl; or
optionally R^{cB} is C₁₋₆ alkyl which is substituted with from 1-6 independently selected R^{a}, such as R^{cB} is CF₃ or 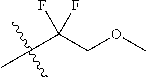 [SMILES: *C(F)(F)COC]
(e.g., R^{c} can be CF₃); and
optionally wherein R^{cA} is an independently selected halo.

204. The compound of any one of claims 202-203, wherein n1 is 0.

205. The compound of any one of claims 202-203, wherein n1 is 1; and R^{cA} is halo.

206. The compound of any one of claims 172-205, wherein L^{AB} is —N(R^{N})S(O)₂—*.

207. The compound of any one of claims 172-205, wherein L^{AB} is —N(R^{N})S(O)₂—(W^{AB1}—W^{AB2}—W^{AB3})—*, such as —N(R^{N})S(O)₂—(C₁₋₃ alkylene)- or —N(R^{N})S(O)₂—(C₁₋₃ alkylene)-O—(C₁₋₃ alkylene).

208. The compound of any one of claims 172-207, wherein R^{N} is H.

209. The compound of claim 172, wherein the compound has Formula (I-1): 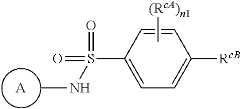
wherein n1=0 or 1; and each of R^{cA} and R^{cB} is an independently selected R^{c}.

210. The compound of claim 172, wherein the compound has Formula (I-2): 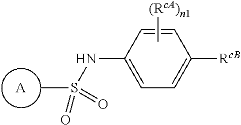
wherein n1=0 or 1; and each of R^{cA} and R^{cB} is an independently selected R^{c}.

211. The compound of claims 209-210, wherein A is (A-1) as defined in claim 173.

212. The compound of any one of claims 209-211, wherein A is: 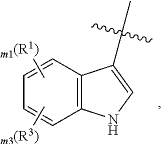
wherein m1=0, 1, 2, or 3; and m3=0, 1, 2, or 3 (e.g., m1=0 or 1; and m3=0, 1, or 2).

213. The compound of any one of claims 209-211, wherein A is A is 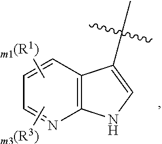
wherein m1=0, 1, or 2; and m3=0, 1, or 2 (e.g., m1=0 or 1; and m3=0 or 1).

214. The compound of any one of claims 212-213, wherein m1=0.

215. The compound of claim 214, wherein m3=1; or wherein m3=2.

216. The compound of claim 215, wherein each occurrence of R³ is independently selected from the group consisting of: halo, cyano, C₁₋₄ alkoxy, C₁₋₄ haloalkoxy, —S(O)₁₋₂(C₁₋₄ alkyl), —NR^{e}R^{f}, —OH, —S(O)₁₋₂(NR′R″), —C₁₋₄ thioalkoxy, —C(═O)(C₁₋₄ alkyl), —C(═O)O(C₁₋₄ alkyl), —C(═O)OH, and —C(═O)N(R′)(R″).

217. The compound of claim 214, wherein m3=0.

218. The compound of any one of claims 209-210, wherein A is (A-2) as defined in claim 183.

219. The compound of any one of claims 209-210, wherein A is as defined in claim 185.

220. The compound of any one of claims 209-210, wherein A is as defined in claim 186.

221. The compound of any one of claims 219-220, wherein m1=0.

222. The compound of any one of claims 219-221, wherein m3=0.

223. The compound of any one of claims 209-210, wherein A is (A-3) as defined in claim 187.

224. The compound of claim 223, wherein A is

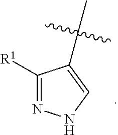

225. The compound of claim any one of claims 223-224, wherein R¹ is phenyl, which is optionally substituted with from 1-2 (e.g., 0; e.g., 1) R^{c}; and optionally wherein each R^{c} substituent of R¹ is independently selected from the group consisting of: halo, cyano, C₁₋₆ alkyl, and C₁₋₄ haloalkyl, such as each R^{c} is an independently selected halo.

226. The compound of any one of claims 209-225, wherein R^{cB} is C₁₋₁₀ alkyl which is optionally substituted with from 1-6 independently selected R^{a}, such as C₁₋₆ alkyl which is optionally substituted with from 1-6 independently selected R^{a};
optionally R^{cB} is unsubstituted C₁₋₁₀ alkyl, such as unsubstituted C₂₋₁₀ (e.g., C₂₋₃, e.g., C₃₋₄, e.g., C₄₋₁₀) alkyl; or
optionally R^{cB} is C₁₋₆ alkyl which is substituted with from 1-6 independently selected R^{a}, such as R^{cB} is CF₃ or 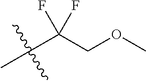 [SMILES: *C(F)(F)COC]
(e.g., R^{c} can be CF₃); and
optionally wherein R^{cA} is an independently selected halo.

227. The compound of any one of claims 209-226, wherein R^{cB} is unsubstituted C₁₋₁₀ alkyl, such as unsubstituted C₂₋₁₀ (e.g., C₂₋₃, e.g., C₃₋₄, e.g., C₄₋₁₀) alkyl.

228. The compound of any one of claims 209-226, wherein R^{cB} is C₁₋₆ alkyl which is substituted with from 1-6 independently selected R^{a}, such as R^{cB} is CF₃ or 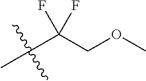 [SMILES: *C(F)(F)COC]
(e.g., R^{c} can be CF₃).

229. The compound of any one of claims 209-228, wherein n1 is 0.

230. The compound of any one of claims 209-228, wherein n1 is 1; and R^{cA} is halo.

231. The compound of claim 172, wherein A is selected from the group consisting of: 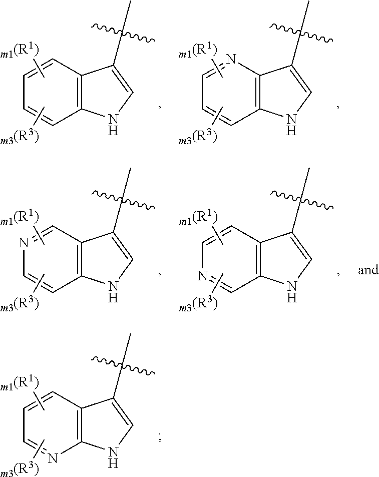
m1 is 0 or 1; and m3 is 0, 1, or 2;
L^{AB} is —N(H)S(O)₂—* and —NHS(O)₂—(W^{AB1})—*; and
B is selected from the group consisting of:
C₆ aryl substituted with from 1-4 R^{c};
heteroaryl including from 5-6 ring atoms, wherein from 1-3 ring atoms are heteroatoms, each independently selected from the group consisting of N, N(H), N(R^{d}), O, and S(O)₀₋₂, and wherein the heteroaryl ring is substituted with from 1-4 independently selected R^{c};
bicyclic or tricyclic heteroaryl including from 9-15 ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c};
C₅₋₁₅ alkyl which is optionally substituted with from 1-6 R^{a}; and
C₈₋₂₀ aryl optionally substituted with from 1-4 R^{c}.

232. The compound of claim 231, wherein m1 is 0.

233. The compound of claim 231, wherein m1 is 1.

234. The compound of any one of claims 231-233, wherein m3 is 0.

235. The compound of any one of claims 231-233, wherein m3 is 1 or 2, such as 2.

236. The compound of claim 231, wherein m1 is 0; and m3 is 2.

237. The compound of claim 236, wherein A is 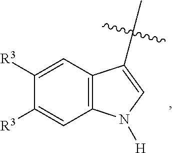
such as

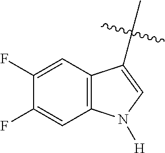 [SMILES: *c1c[nH]c2cc(F)c(F)cc12]

238. The compound of any one of claims 231-237, wherein each occurrence of R³ is independently selected from the group consisting of: halo, cyano, C₁₋₄ alkoxy, C₁₋₄ haloalkoxy, —S(O)₁₋₂(C₁₋₄ alkyl), —S(O)₁₋₂(NR′R″), —C(═O)(C₁₋₄ alkyl), —C(═O)O(C₁₋₄ alkyl), —C(═O)OH, and —C(═O)N(R′)(R″).

239. The compound of any one of claims 231-238, wherein L^{AB} is NHS(O)₂—*.

240. The compound of any one of claims 231-238, wherein L^{AB} is NHS(O)₂—(C₁₋₃ alkylene)-*.

241. The compound of any one of claims 231-240, wherein B is selected from the group consisting of: 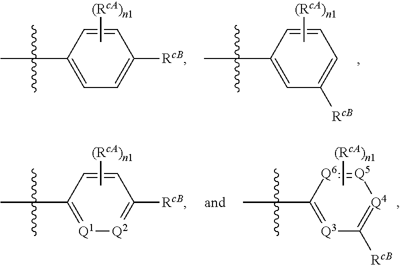
wherein each R^{cA} and R^{cB} is an independently selected R^{c}; n1 is 0, 1, or 2; each of Q¹, Q², Q³, Q⁴, Q⁵, and Q⁶ is independently selected from the group consisting of N and CH, provided that at least one of Q¹ and Q² is N; and at least one of Q³, Q⁴, Q⁵, and Q⁶ is N.

242. The compound of claim 241, wherein n1 is 0.

243. The compound of claim 241, wherein n1 is 1; and R^{cA} is halo (e.g., —F, or —Cl) or C₁₋₆ alkyl which is optionally substituted with from 1-3 independently selected R^{a} (e.g., methyl or CF₃).

244. The compound of any one of claims 241-243, wherein R^{cB} is C₁₋₆ alkyl which is optionally substituted with from 1-6 independently selected R^{a}.

245. The compound of any one of claims 241-243, wherein R^{cB} is -L¹-L²-R^{h}.

246. The compound of any one of claims 231-240, wherein B is heteroaryl including 5 ring atoms, wherein from 1-3 ring atoms are heteroatoms, each independently selected from the group consisting of N, N(H), N(R^{d}), O, and S(O)₀₋₂, and wherein the heteroaryl ring is substituted with from 1-4 independently selected R^{c}, provided that one occurrence of R^{c} is L¹-L²-R^{h}.

247. The compound of any one of claims 245-246, wherein each one of L¹ and L² is a bond.

248. The compound of any one of claims 245-247, wherein R^{h} is selected from the group consisting of:
C₃₋₆ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 substituents independently selected from the group consisting of halo, C₁₋₄ alkyl, and C₁₋₄ haloalkyl; and
C₆ aryl, which is optionally substituted with from 1-4 substituents independently selected from the group consisting of halo, C₁₋₄ alkyl, or C₁₋₄ haloalkyl.

249. The compound of claim 172, wherein the compound is selected from the compounds in Table C1; or a pharmaceutically acceptable salt thereof.

250. A pharmaceutical composition comprising a compound as claimed in any one of claims 172-249.

251. The compound of any one of claims 172-249, wherein the compound exhibits activity as a STING antagonist.
